(19) United States
(12) Patent Application Publication
(10) Pub. No.: US 2009/0286831 A1
(43) Pub. Date: Nov. 19, 2009

(54) VR1 Receptor Ligands and u-Opioid Receptor Ligands for the Treatment of Pain

(75) Inventors: Babette-Yvonne KOEGEL, Langerwehe-Hamrich (DE); Thomas CHRISTOPH, Aachen (DE); Gregor BAHRENBERG, Aachen (DE); Robert FRANK, Aachen (DE); Wolfgang SCHROEDER, Aachen (DE); Jean DE VRY, Stolberg (DE); Eric-Paul PAQUES, Aachen (DE); Derek SAUNDERS, Aachen (DE); Klaus SCHIENE, Duesseldorf (DE); Bernd SUNDERMANN, Aachen (DE); Jeewoo LEE, Ansan-Si (KR); Hyung-Chul RYU, Ansan-Si (KR)

Correspondence Address: CROWELL & MORING LLP;INTELLECTUAL PROPERTY GROUP, P.O. BOX 14300, WASHINGTON, DC, 20044-4300, US

(73) Assignee: Gruenenthal GMBH, Aachen (DE)

(21) Appl. No.: 12/425,721

(22) Filed: Apr. 17, 2009

Related U.S. Application Data
(63) Continuation of application No. PCT/EP2007/009097, filed on Oct. 19, 2007.

(30) Foreign Application Priority Data
Oct. 19, 2006 (EP) 2006/010057
Apr. 16, 2007 (DE) 10 2007 018 150.9

(51) Int. Cl. A61K 31/445; A61P 25/00; A61K 31/4545; A61K 31/4535

(52) U.S. Cl. 514/318; 514/326; 514/329

(57) ABSTRACT

A pharmaceutical composition comprising i) at least one composition that has affinity to the μ-opioid receptor, and at least one compound that has affinity to the VR1 receptor, or ii) at least one compound, in particular at least one compound corresponding to formula (I), (II), (III), (IV) or (V), that has affinity to the μ-opioid receptor and to the VR1 receptor, and also to the use of the pharmaceutical compositions i) and ii) for the production of a drug for the treatment of pain.

### CROSS REFERENCE TO RELATED APPLICATIONS

This application is a continuation of international patent application no. PCT/EP2007/009097, filed Oct. 19, 2007 designating the United States of America, and published in German on Apr. 24, 2008 as WO 2008/046647, the entire disclosure of which is incorporated herein by reference. Priority is claimed based on Federal Republic of Germany patent application no. DE 10 2007 018 150.9, filed Apr. 16, 2007, and on European patent application no. EP 2006/010057, filed Oct. 19, 2006.

### BACKGROUND OF THE INVENTION

This invention relates to a pharmaceutical composition comprising i) at least one composition that has affinity to the μ-opioid receptor, and at least one compound that has affinity to the vanilloid receptor 1 (VR1 receptor), or ii) at least one compound, in particular at least one compound corresponding to formula (I), (II), (III), (IV) or (V), that has affinity to the μ-opioid receptor and to the VR1 receptor, and also to the use of the pharmaceutical compositions i) and ii) for the production of a drug for the treatment of pain.

Besides acute pain, which is of limited duration and generally subsides quickly again after removal of the stimulus triggering it, chronic pain in particular poses a challenge for medical science. Acute pain events as a result of stimulation of intact nociceptors have a warning function to retain body integrity. The subsequent reactions to prevent pain protect against injuries. Chronic pain has lost this protective function. A painful condition is presented. Chronic pain can be divided into two large groups. Pathophysiological nociceptor pain is caused by tissue traumas as a result of stimulation of intact nociceptors. These include in particular chronic inflammatory pain. However, pain that results from damage to the nerves themselves is referred to as neuropathic pain.

The transition from acute pain to chronic pain can occur within hours. Pain treatment during and immediately after an operation is affected by this, for example. Although doctors nowadays consider the treatment of acute pain highly important, the treatment of postoperative pain has significant limitations (Power, Brit. J. Anaesth., 2005, 95, 43-51). Acute pain can become chronic peripherally and in the CNS as a result of pathophysiological processes following tissue damage, e.g. an operation. The association between tissue damage, acute postoperative pain and developing chronic pain has been well researched, and the intensity of acute pain can be regarded as a predictive factor for the duration of chronic pain (Power, Brit. J. Anaesth., 2005, 95, 43-51). A satisfactory treatment of acute pain is essential for this reason alone.

The side-effects of the μ-opioids that are highly effective with respect to acute pain such as morphine or fentanyl, in particular respiratory depression, pose a problem in controlling acute pain. Since this side-effect occasionally causes fatalities in recently operated patients, the drugs are not given in sufficient quantity to satisfactorily control pain in many cases. However, treatment of postoperative pain without opioids is unimaginable nowadays. The fear of respiratory depression and further side-effects typical of μ-opioids, however, in many cases result in opioids not being used to an adequate extent in cases of intense pain, e.g. in cancer patients (Davies et al., Respiratory Care Journal 1999, 44 (1)). Moreover, the risk of respiratory depression occurring after the administration of opioids is increased in older people compared to younger people. In fact, the risk of developing respiratory depression increases significantly in people from 60 years of age (Cepeda et al., Clinical Pharmacology and Therapeutics 2003, 74, 102-112). There is therefore an urgent requirement for new drugs for the treatment of pain, with which respiratory depression is reduced.

However, as already mentioned, the treatment of chronic pain poses a substantial challenge, since while the drugs currently available are highly effective in some instances with respect to acute pain, in many cases they do not result in a satisfactory pain treatment for chronic pain.

Pain occurs as one of the five cardinal symptoms of inflammation besides redness, swelling, heat and impaired function. Inflammatory processes belong to the most important mechanisms of the occurrence of pain. Typical instances of inflammatory pain are triggered by the release of bradykinin, histamine and prostaglandins with acidification of the tissue and pressure of the exudate on the nociceptors. Nociception, unlike other sensations, is not subject to habituation. Instead, previous pain pulses can reinforce the processing of subsequent stimuli in the sense of sensitisation. For example, if an increased influx of pain pulses into the central nervous system occurs as a result of sustained activation of nociceptors in the inflamed tissue, permanent sensitisation phenomena occur at the central synapses. These central sensitisation phenomena are expressed in increased spontaneous activity and more intense responses to stimuli of central neurones, whose receptive fields are also increased (Coderre et al., Pain 1993, 52, 259-285). These changes in the response behaviour of central neurones can contribute to spontaneous pain and to hyperalgesia (increased pain sensation to a noxious stimulus) that are typical for inflamed tissue (Yaksh et al., PNAS 1999, 96, 7680-7686).

One of the most important processes in inflammations is the occurrence of arachidonic acid metabolites. These compounds do not activate nociceptors directly, but reduce the stimulus propagation threshold of the C fibres and thus sensitise them for other stimuli. Non-steroidal antiphlogistics (NSAIDs) in particular have proved effective for the treatment of inflammatory pain, since they block the breakdown of arachidonic acid (Dickensen, A., International Congress and Symposium Series—Royal Society of Medicine 2000, 246, 47-54). However, their application in long-term therapy for chronic pain is restricted by significant undesirable effects in some instances such as gastro-intestinal ulcers or toxic kidney damage.

However, the inhibitory control of stimulus propagation is also important in the treatment of inflammatory pain. μ-opioids are the most important representatives of this class. Chronic pancreatitis, for example, is associated with types of pain that are some of the clinically most difficult to treat pain conditions. The administration of NSAIDs may only slightly reduces the pain, but poses too high a risk because of the increased risk of haemorrhaging. The next step is generally treatment with μ-opioids. Narcotics dependence is widespread among the persons concerned (Vercauteren et al., Acta Anaesthesiologica Belgica 1994, 45, 99-105). Therefore, there is an urgent requirement for compounds that are highly effective with respect to inflammatory pain and have a reduced dependence potential.

Neuropathic pain occurs when peripheral nerves are damaged in a mechanical, metabolic or inflammatory manner. The pain causing agents are primarily identified by the occurrence of spontaneous pain, hyperalgesia and allodynia (pain is already triggered by non-noxious stimuli). As a result of lesions an increased expression of Na⁺ channels occurs and therefore spontaneous activity is caused in the damaged axons and their neighbouring axons (England et al., Neurology 1996, 47, 272-276). The excitability of the neurons is increased and they react to incoming stimuli with an increased discharge frequency. An increased pain sensitivity results that contributes to hyperalgesia and spontaneous pain (Baron, Clin. J. Pain 2000; 16 (2 suppl), 12-20). The pharmacological base therapy for neuropathic pain includes tricyclic antidepressants and anticonvulsant drugs, which are used as monotherapy or also in combination with opioids. These medications generally only provide some pain relief, while freedom from pain is often not achieved. The frequently occurring side-effects often preclude increasing the doses of the drugs to achieve a sufficient alleviation of pain. In fact, a higher dosage of a μ-opioid is often required for satisfactory treatment of neuropathic pain than for the treatment of acute pain, and as a result the side-effects gain even greater significance. This problem is further reinforced by the occurrence of typical μ-opioid tolerance development and the resulting necessity to increase the dosage. In summary, it can be noted that neuropathic pain is currently difficult to treat and is only partially alleviated by high doses of μ-opioids (Saudi Pharm. J. 2002, 10 (3), 73-85). Therefore, there is an urgent requirement for drugs for the treatment of chronic pain, which do not have to be increased in dosage until unacceptable side-effects occur in order to assure a satisfactory pain therapy.

Various other active principles that do not have side-effects typical of μ-opioids have been proposed and developed for the treatment of chronic pain in recent years. Thus, antidepressants that have an analgesic effect besides their mood-enhancing effect, for example, are used in the therapy for moderate to intense chronic pain. However, no active principle has as yet been able to shift μ-opioids from the centre of importance in pain therapy. One of the main reasons is the as yet unattained action intensity of the μ-opioids. However, μ-opioids also have further disadvantages besides respiratory depression.

It has been known for over 100 years that an increased perception of pain is one of the symptoms of opioid withdrawal. Nowadays, the occurrence of pain symptoms is one of the criteria in the diagnosis of opioid withdrawal (Angst et al., Anesthesiology 2006, 104, 570-587). An increasing number of animal and human studies show that under certain circumstances μ-opioids can cause changes in pain sensation that lead to hyperalgesia (sensation of increased pain intensity after painful stimulus). These studies show that the phenomenon of opioid-induced hyperalgesia occurs both in the case of short-term and of chronic opioid administration (Pud et al., Drug and Alcohol Dependence 2006, 218-223). It is known, for example, that patients who are given narcosis with a high opioid content require about three-times as much opioid postoperatively as patients with narcosis using hypnotics. This significant effect also restricts the safe use of μ-opioids, since with the resulting increase in dose necessary the side-effects such as for instance respiratory depression become more significant. However, since the treatment of intense pain without opioids is unimaginable nowadays, there is an urgent requirement for drugs that do not themselves lead to increased pain intensity in patients.

The μ-opioids used for pain therapy such as morphine and fentanyl have a potential for dependence. In many cases, withdrawal symptoms occur when these drugs are discontinued. This side-effect of μ-opioids leads to a substantial restriction in the use of these highly effective pain therapies, because μ-opioids are often not prescribed or taken for fear of dependence. Therefore, there is an urgent requirement for pain therapies that are highly effective and at the same time have a reduced dependence potential compared to μ-opioids.

The typical side-effects of μ-opioids do not have the same intensity in every patient. Thus, there are patients for whom the side-effects are tolerable and others for whom they are a significant problem. On average, however, the side-effects are a problem that has not been solvable so far, although μ-opioids, originally used in the form of natural extract of opium, have long been used for the treatment of pain. The first attempts to synthesise a morphine derivative without dependence potential was made as early as 1874. However, it was evident that the resulting substance—heroin—did not have an improved side-effect profile compared to morphine. Numerous further attempts have been made so far to produce highly effective analgesics with an improved side-effect profile. For instance, oxycodone was synthesised in 1925, methadone in 1946, fentanyl in 1961 and tilidine in 1965. However, it was found that a significant reduction in side-effects was associated with a significant reduction in efficacy. The side-effects typical of μ-opioids are well researched; they can be counteracted using the μ-antagonist naloxone, and therefore are part of the active profile of μ-opioids. So far, there are no drugs that have the same intensity of action as clinically used stage 3 μ-opioids (WHO classification) such as fentanyl, sufentanil, morphine, oxycodone, buprenorphine and hydromorphone and at the same time have a significantly reduced side-effect profile.

In summary, it can be determined that the therapy for moderate to intense pain both in acute and chronic pain is largely based on the use of μ-opioids in spite of their disadvantages. This results above all from the high intensity of action of these compounds. However, the disadvantages are so considerable that many patients are not given the necessary therapy for fear of side-effects—both from their own perceptions and from concerns of the doctor. Therefore, there is an urgent requirement for innovative pain therapies that are based on an active principle that provides the high efficacy of μ-opioids, while also reducing the disadvantages such as dependence, increased pain sensation, respiratory depression and reduced efficacy with respect to chronic pain.

### SUMMARY OF THE INVENTION

Therefore, it was an object of the present invention to find an active principle for drugs, wherein drugs that act according to this principle provide the high efficacy of μ-opioids, but exhibit to a lesser extent the disadvantages, such as dependence, respiratory depression and reduced efficacy with respect to chronic pain, compared to μ-opioids.

These and other objects are achieved by the invention as described and claimed hereinafter.

The invention relates to the use i) of a combination comprising at least one compound that has affinity to the μ-opioid receptor and at least one compound that has affinity to the VR1 receptor, or ii) of at least one compound, in particular at least one compound corresponding to formulas (I), (II), (III), (IV) or (V), that has affinity to the μ-opioid receptor and VR1 receptor, wherein the μ-opioid receptor affinity amounts to ≦5.0 μM (Kᵢ value, human) and the VR1 receptor affinity amounts to ≦5.0 μM (Kᵢ value, human), for the production of a drug for the treatment of pain.

In addition, the invention relates to the use i) of a combination comprising at least one compound that has affinity to the μ-opioid receptor and at least one compound that has affinity to the VR1 receptor, or ii) of at least one compound, in particular at least one compound corresponding to formulas (I), (II), (III), (IV) or (V), that has affinity to the μ-opioid receptor and VR1 receptor, wherein the μ-opioid receptor affinity amounts to ≦5.0 μM (Kᵢ value, human) and the VR1 receptor affinity amounts to ≦5.0 M (Kᵢ value, human), for the production of a drug for the treatment of overactive bladder syndrome, coughing, asthma, chronic obstructive pulmonary disease (COPD) and/or diabetes.

The invention additionally relates to a pharmaceutical composition comprising i) at least one combination comprising i) at least one compound that has affinity to the μ-opioid receptor and at least one compound that has affinity to the VR1 receptor, or ii) at least one compound, in particular at least one compound corresponding to formulas (I), (II), (III), (IV) or (V), that has affinity to the μ-opioid receptor and VR1 receptor, wherein the μ-opioid receptor affinity amounts to ≦5.0 μM (Kᵢ value, human) and the VR1 receptor affinity amounts to ≦5.0 μM (Kᵢ value, human).

It has surprisingly been found that the administration of a combination comprising compounds that have affinity to the VR1 receptor and compounds that have affinity to the μ-opioid receptor or the application of compounds with dual mode of action in the case of pain, in particular acute pain, leads to a significantly increased analgesic effect compared to the single application of a μ-opioid receptor ligand. This result makes it obvious that a superadditive effect results from the application of the compounds in the sense of the invention in the case of acute pain.

Moreover, it has surprisingly been found that the application of the pharmaceutical composition according to the invention in the case of pain, in particular chronic pain, causes an antiallodynic effect not observed when a μ-opioid receptor ligand is administered alone.

Therefore, surprisingly, the application of the combination according to the invention or the compounds with dual mode of action leads to an opioid-saving effect in the treatment of acute and chronic pain conditions.

This has the advantage that on application of the pharmaceutical composition according to the invention the analgesic efficacy is not impaired, while the opioid-specific side-effects are reduced.

Moreover, it has surprisingly been found that the combined administration of VR1 receptor ligands and μ-opioid receptor ligands or the application of a compound with dual mode of action leads to an analgesic effect in the case of chronic neuropathy, i.e. in the case of chronic neuropathic pain. It has been found in this case that said interaction with both receptors leads to a selective inhibition of the illness-induced allodynia without influencing the pain threshold in healthy tissue.

Hence, not only can opioid-specific side-effects be reduced by the combination of VR1 receptor ligands and μ-opioid receptor ligands or by compounds with dual mode of action, but an improved analgesic efficacy can also be achieved in the case of specific chronic neuropathic pain.

In the sense of the description the term “combination according to the invention” is understood to mean the pharmaceutical composition comprising VR1 receptor ligands and μ-opioid receptor ligands used for the treatment of pain.

It is known to persons skilled in the art that this dual mode of action, i.e. the unfolding of the pharmacological effect by binding to the VR1 receptor as well as the μ-opioid receptor, can also be achieved in a single compound, i.e. the compound simultaneously has affinity to the VR1 receptor and affinity to the μ-opioid receptor.

In the sense of the description the term “VR1 receptor ligand” is preferably understood to mean the compounds that act as antagonist, inverse agonist or partial antagonist on the VR1 receptor. In this case, the VR1 receptor ligands preferably exhibit a competitive, non-competitive, uncompetitive or mixed-type inhibition of the VR1 receptor.

The VR1 ligands can be used in the combination according to the invention in the form of a physiologically compatible acid addition salt. Physiologically compatible acid addition salts are understood to be pharmaceutically compatible salts according to the invention that are preferably selected from the salts of hydrochloric acid, hydrobromic acid, sulfuric acid, phosphoric acid, methanesulfonic acid, acetic acid, fumaric acid, maleic acid, succinic acid, lactic acid, citric acid and tartaric acid. Mixtures of the aforementioned acids can possibly also be used for the production of the salts.

If the VR1 receptor ligands carry acid groups such as e.g. —COOH—, —SO₃H— and/or —PO₃H₂— groups, the VR1 receptor ligands can also be present in the combination according to the invention as physiologically compatible salts that are formed by the addition of a suitable base. The following can preferably be used as bases: hydroxides, hydrogencarbonates and/or carbonates of alkali and/or earth alkali metals, preferably sodium hydroxide, potassium hydroxide, sodium hydrogencarbonate, potassium hydrogencarbonate, calcium hydrogencarbonate, magnesium hydrogencarbonate, sodium carbonate, potassium carbonate, calcium carbonate and/or magnesium carbonate. Moreover, unsubstituted and/or substituted amines can also be used as bases. Alkali, earth alkali or ammonium salts of the VR1 receptor ligands are preferred. Mixtures of the aforementioned bases can possibly also be used to produce the salts.

The VR1 receptor affinity of the corresponding compounds of the pharmaceutical composition according to the invention, determined based on the Kᵢ value and preferably measured by in vitro tests on human recombinant VR1 receptors, preferably amounts to ≦5.0 μM, more preferred ≦1.0 μM, further preferred ≦100 nM, most preferred ≦10 nM and in particular ≦1.0 nM.

In the sense of the description compounds that have affinity to the μ-opioid receptor are preferably those ligands that act as agonist, partial agonist or mixed agonist-antagonist on opioid receptors. Those μ-opioid receptor ligands that lead to an agonist effect are particularly preferred.

Compounds that have affinity to the μ-opioid receptor can preferably be selected from the group consisting of morphine, codeine, ethyl morphine, dextromethorphan, dextrorphanol, diacetylmorphine, dihydrocodeine, etorphine, hydrocodone, hydromorphone, levorphanol, N-methylmorphinan, oxycodone, oxymorphone, pentazocine, pholcodine, racemorphan, pethidine, ketobemidone, fentanyl, alfentanil, remifentanil, sufentanil, nefopam, flupirtine, levomethadone, methadone, normethadone, levomethadyl acetate, dextromoramide, dextropropoxyphene, diphenoxylate, loperamide, piritramide, alphaprodine, cetobemidone, tilidine, viminol, buprenorphine, butorphanol, dezocine, meptazinol, nalbuphine and nalorphine.

It is more preferred that the μ-opioid receptor ligands can be selected from the group consisting of morphine, hydromorphone, oxycodone, oxymorphone, pentazocine, pethidine, fentanyl, alfentanil, sufentanil, methadone, tilidine and buprenorphine.

It is further preferred that the μ-opioid receptor ligands can be selected from the group consisting of morphine, oxymorphone, fentanyl, methadone, tilidine and buprenorphine, wherein morphine is considered in particular.

The μ-opioid receptor ligands can be used in the combination according to the invention in the form of a physiologically compatible acid addition salt. Physiologically compatible acid addition salts are understood to be salts that are pharmaceutically compatible according to the invention preferably selected from the salts of hydrochloric acid, hydrobromic acid, sulfuric acid, phosphoric acid, methanesulfonic acid, acetic acid, fumaric acid, succinic acid, lactic acid, citric acid, tartaric acid and maleic acid. Mixtures of the aforementioned acids can possibly also be used for the production of the salts.

The salts of the μ-opioid receptor ligands selected from the group consisting of hydrochloride, hydrobromide, sulfate, phosphate, fumarate and methanesulfonate are preferred according to the invention. The salts selected from hydrochloride, hydrobromide and fumarate are particularly preferred, wherein hydrochloride is considered the most significant according to the invention. The μ-opioid receptor ligand may possibly be present in the form of a hydrate.

Like the VR1 ligands, the μ-opioid receptor ligands can also be incorporated into the combination according to the invention as mixtures of their corresponding configuration isomers, i.e. cis- and/or trans-isomers and/or stereoisomers, i.e. diastereomers, epimers and/or enantiomers.

The μ-opioid receptor affinity, determined based on the Kᵢ value and preferably measured by in vitro tests, preferably amounts to ≦5.0 μM, more preferred ≦1.0 μM, further preferred ≦100 nM, most preferred ≦10 nM and in particular ≦1.0 nM. The corresponding in vitro tests for the determination of the Kᵢ value are known to the skilled person from specialist literature.

As described above, the combination according to the invention can also comprise those compounds that exhibit both modes of action (dual mode of action), i.e. the compounds have both VR1 receptor affinity and μ-opioid receptor affinity. These compounds can be used in the composition according to the invention together with other VR1 receptor ligands and/or μ-opioid receptor ligands and/or other compounds with dual mode of action.

The compounds that exhibit this dual mode of action can also be incorporated into the combination according to the invention as mixtures of their corresponding configuration isomers, i.e. cis- and/or trans-isomers and/or stereoisomers, i.e. diastereomers, epimers and/or enantiomers.

Compounds with dual mode of action are preferably such compounds corresponding to formulas (I), (II), (III), (IV) and (V), i.e. they have an affinity to the μ-opioid receptor in addition to an affinity to the VR1 receptor.

In a preferred embodiment at least one compound corresponding to formula (I) can be used to produce a drug for the treatment of pain:

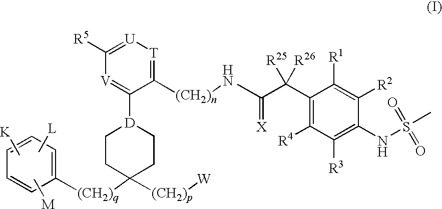

wherein
D is CH or N;
W stands for —CN, —NR³⁴R³⁵, —C(═O)—R³⁶ or —C(═O)—OR³⁷;
X stands for O, S or N—C≡N;
n is 0, 1, 2, 3 or 4;
p is 0, 1, 2 or 3;
q is 0, 1, 2 or 3;
T stands for C—R⁶ and U for C—R⁷ and V for N;
or
T stands for C—R⁶ and U for N and V for C—R⁹;
or
T stands for N and U for C—R⁷ and V for C—R⁹;
or
T stands for N and U for N and V for C—R⁹;
or
T stands for N and U for C—R⁷ and V for N;
or
T stands for C—R⁶ and U for N and V for N;
or
T stands for C—R⁶ and U for C—R⁷ and V for C—R⁹;
R¹, R², R³ and R⁴ each independently stand for H; F; Cl; Br; I; —SF₅; —NO₂; —CF₃; —CN; —NH₂; —OH; —SH; —C(═O)—NH₂; —S(═O)₂—NH₂; —C(═O)—NH—OH; —C(═O)—OH; —C(═O)—H; —S(═O)₂—OH; —NHR¹¹; —NR¹²R¹³; —OR¹⁴; —SR¹⁵; —C(═O)—NHR¹⁶; —C(═O)—NR¹⁷R¹⁸; —S(═O)₂—NHR¹⁹; —S(═O)₂—NR²⁰R²¹; —C(═O)—OR²²; —C(═O)—R²³; —S(═O)—R²⁴; —S(═O)₂—R²⁴, or for a linear or branched, saturated or unsaturated, unsubstituted or mono- or polysubstituted aliphatic C₁₋₁₀ residue;
R⁵ stands for H; F; Cl; Br; I; —SF₅; —NO₂; —CF₃; —CF₂CT; —CN; —NH₂; —OH; —SH; —C(═O)—NH₂; —S(═O)₂—NH₂; —C(═O)—NH—OH; —C(═O)—OH; —C(═O)—H; —S(═O)₂—OH; —NHR¹¹; —NR¹²R¹³; —OR¹⁴; —SR¹⁵; —C(═O)—NHR¹⁶; —C(═O)—NR¹⁷R¹⁸; —S(═O)₂—NHR¹⁹; —S(═O)₂—NR²⁰R²¹; —C(═O)—OR²²; —C(═O)—R²³, —S(═O)₂—R²⁴; —S(═O)—R²⁴; or
   for a linear or branched, saturated or unsaturated, unsubstituted or mono- or polysubstituted aliphatic C₁₋₁₀ residue; or
   for an unsaturated or saturated, unsubstituted or mono- or polysubstituted 3-, 4-, 5-, 6-, 7-, 8- or 9-membered cycloaliphatic residue, which optionally may have one or more heteroatoms as ring members and is respectively bonded to the basic framework via a carbon atom in the ring of the cycloaliphatic residue;
R⁶ and R⁷ each independently stand for H; F; Cl; Br; I; —SF₅; —NO₂; —CF₃; —CF₂Cl; —CN; —NH₂; —OH; —SH; —C(═O)—NH₂; —S(═O)₂—NH₂; —C(═O)—NH—OH; —C(═O)—OH; —C(═O)—H; —S(═O)₂—OH; —NHR¹¹; —NR¹²R¹³; —OR¹⁴; —SR¹⁵; —C(═O)—NHR¹⁶; —C(═O)—NR¹⁷R¹⁸; —S(═O)₂—NHR¹⁹; —S(═O)₂—NR²⁰R²¹; —C(═O)—OR²²; —C(═O)—R²³, —S(═O)—R²⁴; —S(═O)₂OR²⁴;
   for a linear or branched, saturated or unsaturated, unsubstituted or mono- or polysubstituted aliphatic C₁₋₁₀ residue; or
   for an unsubstituted or mono- or polysubstituted 6- or 10-membered aryl residue, which can be bonded via a linear or branched, unsubstituted or mono- or polysubstituted C₁₋₆-alkylene group or C₂₋₆-alkenylene group or C₂₋₆-alkinylene group;
R⁹ stands for H; F; Cl; Br; I; —SF₅; —NO₂; —CF₃; —CF₂Cl; —CN; —NH₂; —OH; —SH; —C(═O)—NH₂; —S(═O)₂—NH₂; —C(═O)—NH—OH; —C(═O)—OH; —C(═O)—H; —S(═O)₂—OH; —NHR¹¹; —NR¹²R¹³; —OR¹⁴; —SR¹⁵; —C(═O)—NHR¹⁶; —C(═O)—NR¹⁷R¹⁸; —S(═O)₂—NHR¹⁹; —S(═O)₂—NR²⁰R²¹; —C(═O)—OR²²; —C(═O)—R²³; —S(═O)—R²⁴; —S(═O)₂—R²⁴ or for a linear or branched, saturated or unsaturated, unsubstituted or mono- or polysubstituted aliphatic C₁₋₁₀ residue;
R¹¹, R¹², R¹³, R¹⁴, R¹⁵, R¹⁶, R¹⁷, R¹⁸, R¹⁹, R²⁰, R²¹, R²², R²³ and R²⁴ each independently stand for a linear or branched, saturated or unsaturated, unsubstituted or mono- or polysubstituted aliphatic C₁₋₁₀ residue; or
   for an unsaturated or saturated, unsubstituted or mono- or polysubstituted 3-, 4-, 5-, 6-, 7-, 8- or 9-membered cycloaliphatic residue, which optionally may contain one or more heteroatoms as ring members and which optionally may be condensed with a saturated or unsaturated, unsubstituted or mono- or polysubstituted, mono- or polycyclic ring system and/or can be bonded via a linear or branched, unsubstituted or mono- or polysubstituted C₁₋₆-alkylene group or 2- to 6-membered heteroalkylene group; or
   for an unsubstituted or mono- or polysubstituted 5- to 14-membered aryl or heteroaryl residue, which optionally may be condensed with a saturated or unsaturated, unsubstituted or mono- or polysubstituted mono- or polycyclic ring system and/or may be bonded via a linear or branched, unsubstituted or mono- or polysubstituted C₁₋₆-alkylene group or 2- to 6-membered heteroalkylene group; or
R¹² and R¹³ together with the nitrogen atom connecting them form a saturated or unsaturated, unsubstituted or mono- or polysubstituted 4-, 5-, 6-, 7-, 8- or 9-membered heterocycloaliphatic residue, which optionally may contain one or more further heteroatoms as ring members and which optionally may be condensed with a saturated or unsaturated, unsubstituted or mono- or polysubstituted mono- or polycyclic ring system;
K, L and M each independently stand for H, F, Cl, Br, I, —CN, —CF₃, —SF₅, —OH, —O—C₁₋₅-alkyl, —NH₂, —NO₂, —O—CF₃, —S—CF₃, —SH, —S—C₁₋₅-alkyl, —C₁₋₅-alkyl, —C(═O)—OH, —C(═O)—O—C₁₋₅-alkyl, —NH—C₁₋₅-alkyl, —N(C₁₋₅-alkyl)₂, —NH—S(═O)₂—C₁₋₅-alkyl, —NH—C(═O)—O—C₁₋₅-alkyl, —C(═O)—H, —C(═O)—C₁₋₅-alkyl, —C(═O)—NH₂, —C(═O)—NH—C₁₋₅-alkyl, —C(═O)—N—(C₁₋₅-alkyl)₂, —O-phenyl, —O-benzyl, phenyl and benzyl, wherein the cyclic part of the residues —O-phenyl, —O-benzyl, phenyl and benzyl can be respectively substituted with 1, 2, 3, 4 or 5 substituents independently selected from the group consisting of F, Cl, Br, —OH, —CF₃, —SF₅, —CN, —NO₂, —C₁₋₅-alkyl, —O—C₁₋₅-alkyl, —O—CF₃, —S—CF₃, phenyl and —O-benzyl;
R²⁵ and R²⁶ each independently stand for hydrogen; or
   for a linear or branched, saturated or unsaturated, unsubstituted or mono- or polysubstituted aliphatic C₁₋₁₀ residue; or
   for an unsubstituted or mono- or polysubstituted 5- to 14-membered aryl or heteroaryl residue, which optionally may be condensed with a saturated or unsaturated, unsubstituted or mono- or polysubstituted mono- or polycyclic ring system and/or may be bonded via a linear or branched, unsubstituted or mono- or polysubstituted C₁₋₆-alkylene group or C₂₋₆-alkenylene group or C₂₋₆-alkinylene group; or
   for an unsaturated or saturated, unsubstituted or mono- or polysubstituted 3-, 4-, 5-, 6-, 7-, 8- or 9-membered cycloaliphatic residue, which optionally may contain one or more heteroatoms as ring members;
with the proviso that R²⁵ and R²⁶ do not simultaneously stand for hydrogen; or
R²⁵ and R²⁶ together with the carbon atom connecting them form a saturated or unsaturated, unsubstituted or mono- or polysubstituted 3-, 4-, 5- or 6-membered cycloaliphatic residue; and
R³⁴, R³⁵, R³⁶ and R³⁷ each independently stand for hydrogen, or for a linear or branched, saturated or unsaturated aliphatic C₁₀ residue; or
   for an unsaturated or saturated, unsubstituted or mono- or polysubstituted 3-, 4-, 5-, 6-, 7-, 8- or 9-membered cycloaliphatic residue, which is respectively bonded to the basic framework via a carbon atom in the ring of the cycloaliphatic residue and which optionally may be condensed with a saturated or unsaturated, unsubstituted or mono- or polysubstituted mono- or polycyclic ring system and/or optionally may be bonded via a linear or branched, unsubstituted or mono- or polysubstituted C₁₋₆-alkylene group or C₂₋₆-alkenylene group or C₂₋₆-alkinylene group; or
   for an unsubstituted or mono- or polysubstituted 5- to 14-membered aryl or heteroaryl residue, which optionally may be condensed with a saturated or unsaturated, unsubstituted or mono- or polysubstituted mono- or polycyclic ring system and/or may be bonded via a linear or branched, unsubstituted or mono- or polysubstituted C₁₋₆-alkylene group or C₂₋₆-alkenylene group or C₂₋₆-alkinylene group;
      in the form of one of its individual stereoisomers, in particular enantiomers or diastereomers, its racemates or in the form of a mixture of stereoisomers, in particular enantiomers and/or diastereomers, in any desired mixture ratio, or respectively in the form of corresponding salts, or respectively in the form of corresponding solvates.

Unless otherwise specified, aliphatic C₁₋₁₀ residues can preferably be respectively optionally substituted with 1, 2, 3, 4, 5, 6, 7, 8 or 9 substituents independently selected from the group consisting of —C(═O)—O—C₁₋₅-alkyl, —O—C(═O)—C₁₋₅-alkyl, —O-phenyl, phenyl, F, Cl, Br, I, —CN, —NO₂, —OH, —NH₂, —SH, —O(C₁₋₅-alkyl), —S(C₁₋₅-alkyl), —NH(C₁₋₅-alkyl), —N(C₁₋₅-alkyl)(C₁₋₅-alkyl), —OCF₃ and —SCF₃.

The C₁₋₆-alkylene groups, C₂₋₆-alkenylene groups and C₂₋₆-alkinylene groups can preferably be respectively optionally substituted with 1, 2, 3, 4, 5, 6, 7, 8 or 9 substituents independently selected from the group consisting of F, Cl, Br, I, —CN, —NO₂, —OH, —NH₂, —SH, —O(C₁₋₅-alkyl), —S(C₁₋₅-alkyl), —NH(C₁₋₅-alkyl), —N(C₁₋₅-alkyl)(C₁₋₅-alkyl), —OCF₃ and —SCF₃.

The term “heteroalkylene” refers to an alkylene chain, in which one or more carbon atoms have respectively been replaced by a heteroatom independently selected from the group consisting of oxygen, sulfur and nitrogen (NH). Heteroalkylene groups can preferably have 1, 2 or 3 heteroatom(s), particularly preferred one heteroatom, as chain member(s) independently selected from the group consisting of oxygen, sulfur and nitrogen (NH). Hetereoalkylene groups can preferably be 2- to 6-membered, particularly preferably 2- or 3-membered. Examples of heteroalkylene groups include —CH₂—CH₂—O—CH₂—, —CH₂—CH(CH₃)—O—CH₂—, —(CH₂)—O—, —(CH₂)₂—O—, —(CH₂)₃—O—, —(CH₂)₄—O—, —O—(CH₂)—, —O—(CH₂)₂—, —O—(CH₂)₃—, —O—(CH₂)₄—, —C(C₂H₅)(H)—O—, —O—C(C₂H₅)(H)—, —CH₂—O—CH₂—, —CH₂—S—CH₂—, —CH₂—NH—CH₂—, —CH₂—NH— and —CH₂—CH₂—NH—CH₂—CH₂.

The 2- to 6-membered heteroalkylene groups can preferably be respectively optionally substituted with 1, 2, 3, 4, 5, 6, 7, 8 or 9 substituents independently selected from the group consisting of F, Cl, Br, I, —CN, —NO₂, —OH, —NH₂, —SH, —O(C₁₋₅-alkyl), —S(C₁₋₅-alkyl), —NH(C₁₋₅-alkyl), —N(C₁₋₅-alkyl)(C₁₋₅-alkyl), —OCF₃ and —SCF₃.

(Hetero)cycloaliphatic residues can preferably be respectively optionally substituted with 1, 2, 3, 4 or 5 substituents independently selected from the group consisting of —C₁₋₆-alkylene-OH, ═CH₂, —O—C₁₋₅-alkylene-oxetanyl, —C₁₋₅-alkylene-O—C₁₋₅-alkylene-oxetanyl, —CH₂—NH—C₁₋₅-alkyl, —CH₂—N(C₁₋₅-alkyl)₂, —N[C(═O)—C₁₋₅-alkyl]-phenyl, —CH₂—O—C₁₋₅-alkyl, oxo (═O), thioxo (═S), F, Cl, Br, I, —CN, —CF₃, —SF₅, —OH, —O—C₁₋₅-alkyl, —O—C(═O)—C₁₋₅-alkyl, —NH₂, —NO₂, —O—CF₃, —S—CF₃, —SH, —S—C₁₋₅-alkyl, —C₁₋₅-alkyl, —C(═O)—C₁₋₅-alkyl, —C(═O)—OH, —C(═O)—O—C₁₋₅-alkyl, —NH—C₁₋₅-alkyl, —N(C₁₋₅-alkyl)₂, (4,5)-dihydroisoxazolyl, thiazolyl, (1,2,5)-thiadiazolyl, thiophenyl, phenethyl, —NH-phenyl, —N(C₁₋₅-alkyl)-phenyl, cyclohexyl, cyclopentyl, piperidinyl, pyrrolidinyl, —(CH₂)-pyridinyl, pyridinyl, —O-phenyl, —O-benzyl, phenyl and benzyl, wherein the cyclic part of the oxetanyl, (4,5)-dihydroisoxazolyl, thiazolyl, (1,2,5)-thiadiazolyl, thiophenyl, phenethyl, —N[C(═O)—C₁₋₅-alkyl]-phenyl, —NH-phenyl, —N(C₁₋₅-alkyl)-phenyl, —(CH₂)-pyridinyl, pyridinyl, —O-phenyl, —O-benzyl, phenyl and benzyl residues can be respectively substituted with 1, 2, 3, 4 or 5 substituents independently selected from the group consisting of F, Cl, Br, —OH, —CF₃, —SF₅, —CN, —NO₂, —C₁₋₅-alkyl, —O—C₁₋₅-alkyl, —O—CF₃, —S—CF₃, phenyl and —O-benzyl.

It is also preferred that (hetero)cycloaliphatic residues can respectively optionally contain 1, 2 or 3 (further) heteroatom(s) independently selected from the group consisting of oxygen, nitrogen and sulfur.

The rings of the mono- or polycyclic ring systems can preferably be respectively optionally substituted with 1, 2, 3, 4 or 5 substituents independently selected from the group consisting of oxo (═O), thioxo (═S), F, Cl, Br, I, —CN, —CF₃, —SF₅, —OH, —O—C₁₋₅-alkyl, —NH₂, —NO₂, —O—CF₃, —S—CF₃, —SH, —S—C₁₋₅-alkyl, —C₁₋₅-alkyl, —C(═O)—C₁₋₅-alkyl, —C(═O)—OH, —C(═O)—O—C₁₋₅-alkyl, —NH—C₁₋₅-alkyl, —N(C₁₋₅-alkyl)₂, —O-phenyl, —O-benzyl, phenyl and benzyl, wherein the cyclic part of the residues —O-phenyl, —O-benzyl, phenyl and benzyl can be respectively substituted with 1, 2, 3, 4 or 5 substituents independently selected from the group consisting of F, Cl, Br, —OH, —CF₃, —SF₅, —CN, —NO₂, —C₁₋₅-alkyl, —O—C₁₋₅-alkyl, —O—CF₃, —S—CF₃, phenyl and —O-benzyl.

The rings of the mono- or polycyclic ring systems are preferably respectively 5-, 6- or 7-membered and can respectively have possibly 1, 2, 3, 4 or 5 heteroatom(s) as ring member(s) independently selected from the group consisting of oxygen, nitrogen and sulfur.

It is also preferred that the aryl or heteroaryl residues can be respectively optionally substituted with 1, 2, 3, 4 or 5 substituents independently selected from the group consisting of F, Cl, Br, I, —CN, —CF₃, —SF₅, —OH, —O—C₁₋₅-alkyl, —NH₂, —NO₂, —O—CF₃, —S—CF₃, —SH, —S—C₁₋₅-alkyl, —C₁₋₅-alkyl, —C(═O)—OH, —C(═O)—O—C₁₋₅-alkyl, —NH—C₁₋₅-alkyl, —N(C₁₋₅-alkyl)₂, —NH—S(═O)₂—C₁₋₅-alkyl, —NH—C(═O)—O—C₁₋₅-alkyl, —C(═O)—H, —C(═O)—C₁₋₅-alkyl, —C(═O)—NH₂, —C(═O)—NH—C₁₋₅-alkyl, —C(═O)—N—(C₁₋₅-alkyl)₂, —O-phenyl, —O-benzyl, phenyl and benzyl, wherein the cyclic part of the residues —O-phenyl, —O-benzyl, phenyl and benzyl can be respectively substituted with 1, 2, 3, 4 or 5 substituents independently selected from the group consisting of F, Cl, Br, —OH, —CF₃, —SF₅, —CN, —NO₂, —C₁₋₅-alkyl, —O—C₁₋₅-alkyl, —O—CF₃, —S—CF₃, phenyl and —O-benzyl.

It is also preferred that heteroaryl residues respectively have possibly 1, 2, 3, 4 or 5 heteroatom(s) as ring member(s) independently selected from the group consisting of oxygen, nitrogen and sulfur.

Where one or more residues stand for a saturated or unsaturated C₁₋₁₀ aliphatic residue, i.e. for a C₁₋₁₀ alkyl, C₂₋₁₀ alkenyl or C₂₋₁₀ alkinyl residue, this can preferably be optionally substituted with 1, 2, 3, 4, 5, 6, 7, 8 or 9 substituents independently selected from the group consisting of —O-phenyl, F, Cl, Br, I, —CN, —NO₂, —OH, —NH₂, —SH, —O(C₁₋₅-alkyl), —S(C₁₋₅-alkyl), —NH(C₁₋₅-alkyl), —N(C₁₋₅-alkyl)(C₁₋₅-alkyl), —C(═O)—O—C₁₋₅-alkyl, —OCF₃ and —SCF₃. C₂₋₁₀ alkenyl residues have at least one, preferably 1, 2, 3 or 4 C—C double bonds and C₂₋₁₀ alkinyl residues have at least one, preferably 1, 2, 3 or 4 C—C triple bonds.

Alkyl residues are preferably selected from the group consisting of methyl, ethyl, n-propyl, isopropyl, n-butyl, sec-butyl, isobutyl, tert-butyl, n-pentyl, 3-methyl-but-1-yl, 2-pentyl, 3-pentyl, sec-pentyl, neo-pentyl, 4-methyl-pent-1-yl, (3,3)-dimethyl-but-1-yl, n-hexyl, n-heptyl, 2-heptyl, 3-heptyl, 4-heptyl, n-octyl, n-nonyl, 2-nonyl, 3-nonyl, 4-nonyl, 5-nonyl and (2,6)-dimethyl-hept-4-yl, which can possibly be substituted with 1, 2, 3, 4, 5, 6, 7, 8 or 9 substituents independently selected from the group consisting of —O-phenyl, —O—C(═O)—CH₃, —O—C(═O)—C₂H₅, —O—C(═O)—CH(CH₃)₂, —O—C(═O)—C(CH₃)₃, —C(═O)—O—CH₃, —C(═O)—O—C₂H₅, —C(═O)—O—CH(CH₃)₂, —C(═O)—O—C(CH₃)₃, F, Cl, Br, I, —CN, —NO₂, —OH, —NH₂, —SH, —O—CH₃, —O—C₂H₅, —O—CH(CH₃)₂, —O—C(CH₃)₃, —S—CH₃, —S—C₂H₅, —S—CH(CH₃)₂, —S—C(CH₃)₃, —NH—CH₃, —NH—C₂H₅, —NH—C(CH₃)₃, —N(CH₃)₂, —N(C₂H₅)₂, —N(CH₃)(C₂H₅), —OCF₃ and —SCF₃.

Also preferred are alkenyl residues selected from the group consisting of vinyl, 1-propenyl, 2-propenyl, 1-butenyl, 2-butenyl, 3-butenyl, 2-methyl-propen-1-yl, 3-methyl-but-2-en-1-yl, (3,3)-dimethyl-but-1-enyl, 2-methyl-buten-2-yl, 1-pentenyl, 2-pentenyl, 3-pentenyl, 4-pentenyl, 1-hexenyl, 1-heptenyl and 1-octenyl, which optionally may be substituted with 1, 2 or 3 substituents independently selected from the group consisting of F, Cl, Br, I, —CN, —NO₂, —OH, —NH₂, —SH, —O—CH₃, —O—C₂H₅, —O—CH(CH₃)₂, —O—C(CH₃)₃, —S—CH₃, —S—C₂H₅, —S—CH(CH₃)₂, —S—C(CH₃)₃, —NH—CH₃, —NH—C₂H₅, —NH—C(CH₃)₃, —N(CH₃)₂, —N(C₂H₅)₂, —N(CH₃)(C₂H₅), —OCF₃ and —SCF₃.

Further preferred are alkinyl residues selected from the group consisting of (3,3)-dimethyl-but-1-inyl, 4-methyl-pent-1-inyl, 1-hexinyl, ethinyl, 1-propinyl, 2-propinyl, 1-butinyl, 2-butinyl, 3-butinyl, 1-pentinyl, 2-pentinyl, 3-pentinyl and 4-pentinyl, which optionally may be substituted with 1, 2 or 3 substituents independently selected from the group consisting of F, Cl, Br, I, —CN, —NO₂, —OH, —NH₂, —SH, —O—CH₃, —O—C₂H₅, —O—CH(CH₃)₂, —O—C(CH₃)₃, —S—CH₃, —S—C₂H₅, —S—CH(CH₃)₂, —S—C(CH₃)₃, —NH—CH₃, —NH—C₂H₅, —NH—C(CH₃)₃, —N(CH₃)₂, —N(C₂H₅)₂, —N(CH₃)(C₂H₅), —OCF₃ and —SCF₃.

Particularly preferred optionally substituted C₁₋₁₀ aliphatic residues are selected from the group consisting of methyl, —CF₃, —CHF₂, —CH₂F, —CF₂Cl, —CCl₂F, —CCl₃, —CBr₃, —CH₂—CN, —CH₂—O—CH₃, —CH₂—O—CF₃, —CH₂—SF₃, —CH₂—NH₂, —CH₂—OH, —CH₂—SH, —CH₂—NH—CH₃, —CH₂—N(CH₃)₂, —CH₂—N(C₂H₅)₂, —CH₂—N(CH₃)(C₂H₅), ethyl, —CF₂—CH₃, —CHF—CF₂Cl, —CF₂—CFCl₂, —CFCl—CF₂Cl, —CFCl—CFCl₂, —CH₂—CH₂—NH₂, —CH₂—CH₂—OH, —CH₂—CH₂—SH, —CH₂—CH₂—NH—CH₃, —CH₂—CH₂—N(CH₃)₂, —CH₂—CH₂—N(C₂H₅)₂, —CH₂—CH₂—N(CH₃)(C₂H₅), —CH₂—CF₃, —C₂F₅, —CH₂—CCl₃, —CH₂—CBr₃, —CH₂—CH₂—CN, n-propyl, —CH₂—CH₂—CH₂—OH, —CH₂—CH₂—CH₂—SH, —CH₂—CH₂—CH₂—NH₂, —CH₂—CH₂—CH₂—NH—CH₃, —CH₂—CH₂—CH₂—N(CH₃)₂, —CH₂—CH₂—CH₂—N(C₂H₅)₂, —CH₂—CH₂—CH₂—N(CH₃)(C₂H₅), —CH₂—CH₂—O—CH₃, —CF₂—CF₂—CF₃, —CF(CF₃)₂, isopropyl, —CH₂—CH₂—CH₂—CN, —CH₂—O—CH₂—CH₃, —CH₂—CH₂—SF₃, —CH₂—CH₂—OCF₃, —CH(CH₃)(O—CH₃), —CH(CH₃)(S—CH₃), n-butyl, —CF₂—CF₂—CF₂—CF₃, —CH₂—CH₂—CH₂—CH₂—CN, —CH₂—CH₂—CH₂—CF₃, —CH₂—CH₂—CH₂—CH₂—CF₃, —CH₂—O—C(═O)—CH₃, —CH₂—O—C(═O)—C₂H₅, —CH₂—O—C(═O)—CH(CH₃)₂, —CH₂—O—C(═O)—C(CH₃)₃, —CH₂—C(═O)—O—CH₃, —CH₂—C(═O)—O—C₂H₅, —CH₂—C(═O)—O—C(CH₃)₃, —CH₂—CH₂—O—CH₃, —CH₂—CH₂—O—C₂H₅, —CH₂—CH₂—O-phenyl, —CH₂—CH₂—CH₂—O—CH₃, sec-butyl, isobutyl, tert-butyl, n-pentyl, sec-pentyl, neo-pentyl, n-hexyl, vinyl, 1-propenyl, 2-propenyl, 1-butenyl, 2-butenyl, 3-butenyl, 2-methyl-buten-2-yl, (1,1,2)-trifluoro-1-butenyl, 1-pentenyl, 2-pentenyl, 3-pentenyl, 4-pentenyl, —CF═CF₂, —CCl═CCl₂, —CH₂—CF═CF₂, —CH₂—CCl═CCl₂, —C≡C—I, —C≡C—F and —C≡C—Cl.

If one or more of the substituents stand for a (hetero)cycloaliphatic residue, which optionally may be condensed with a saturated or unsaturated, unsubstituted or mono- or polysubstituted mono- or polycyclic ring system, this can preferably be selected from the group consisting of cyclopropyl, cyclobutyl, cyclopentyl, cyclohexyl, cycloheptyl, cyclopentenyl, cyclohexenyl, cycloheptenyl, imidazolidinyl, tetrahydrofuranyl, tetrahydrothiophenyl, pyrrolidinyl, piperidinyl, morpholinyl, piperazinyl, thiomorpholinyl, tetrahydropyranyl, oxetanyl, (1,2,3,6)-tetrahydropyridinyl, azepanyl, azocanyl, diazepanyl, dithiolanyl, (1,3,4,5)-tetrahydropyrido[4,3-b]indolyl, (3,4)-dihydro-1H-isoquinolinyl, (1,3,4,9)-tetrahydro-[b]-carbolinyl and (1,3)-thiazolidinyl.

Suitable (hetero)cycloaliphatic residues that can be unsubstituted or mono- or polysubstituted and condensed with a mono- or bicyclic ring system are, for example, (4,5,6,7)-tetrahydroisoxazolo[5,4-c]pyridinyl, (2,3)-dihydro-1H-indenyl, 3-aza-bicyclo[3.1.1]heptyl, 3-aza-bicyclo[3.2.1]octyl, 6-aza-bicyclo[3.3.1]heptyl, 8-aza-bicyclo[3.2.1]octyl, isoindolyl, indolyl, (1,2,3,4)-tetrahydroquinolinyl, (1,2,3,4)-tetrahydroisoquinolinyl, (2,3)-dihydro-1H-isoindolyl, (1,2,3,4)-tetrahydronaphthyl, (2,3)-dihydro-benzo[1.4]dioxinyl, benzo[1.3]dioxolyl, (1,4)-benzodioxanyl, (2,3)-dihydrothieno[3,4-b][1,4]dioxinyl, (3,4)-dihydro-2H-benzo[1.4]oxazinyl, octahydro-1H-isoindolyl and octahydro-pyrrolo[3,4-c]pyrrolyl.

In the sense of the present invention, (hetero)cycloaliphatic residues can form a spirocyclic residue with a further (hetero)cycloaliphatic residue via a common carbon atom in both rings. Examples of suitable spirocyclic residues include a 6-aza-spiro[2.5]octyl residue, an 8-azaspiro[4.5]decyl residue and a 1-oxa-2,8-diaza-spiro[4.5]dec-2-enyl residue.

It is particularly preferred that the (hetero)cycloaliphatic residues optionally may be substituted respectively with 1, 2, 3, 4 or 5 substituents independently selected from the group consisting of oxo (═O), thioxo (═S), F, Cl, Br, I, —CN, —CF₃, —SF₅, —OH, —O—CH₃, —O—C₂H₅, —O—CH(CH₃)₂, —O—C(CH₃)₃, —NH₂, —NO₂, —O—CF₃, —S—CF₃, —SH, —S—CH₃, —S—C₂H₅, —S—CH(CH₃)₂, —S—C(CH₃)₃, methyl, ethyl, n-propyl, isopropyl, n-butyl, sec-butyl, isobutyl, tert-butyl, n-pentyl, —C(═O)—CH₃, —C(═O)—C₂H₅, —C(═O)—CH(CH₃)₂, —C(═O)—C(CH₃)₃, —C(═O)—OH, —C(═O)—O—CH₃, —C(═O)—O—C₂H₅, —C(═O)—O—CH(CH₃)₂, —C(═O)—O—C(CH₃)₃, —NH—CH₃, —NH—C₂H₅, —NH—C(CH₃)₃, —N(CH₃)₂, —N(C₂H₅)₂, —N(CH₃)(C₂H₅), —CH₂—OH, —CH₂—CH₂—OH, ═CH₂, —CH₂—O—CH₂-oxetanyl, —O—CH₂-oxetanyl, —CH₂—N(CH₃)₂, —CH₂—N(C₂H₅)₂, —CH₂—NH—CH₃, —CH₂—NH—C₂H₅, —N—[C(═O)—C₂H₅]-phenyl, —N—[C(═O)—CH₃]-phenyl, —CH₂—O—CH₃, —CH₂—O—CH₂—CH₃, —NH-phenyl, —N(CH₃)-phenyl, —N(C₂H₅)-phenyl, —N(C₂H₅)-phenyl, —O—CH₂—CH₂—CH₂—CH₃, (4,5)-dihydroisoxazolyl, thiazolyl, (1,2,5)-thiadiazolyl, thiophenyl, phenethyl, cyclohexyl, cyclopentyl, piperidinyl, pyrrolidinyl, —O—C(═O)—CH₃, —O—C(═O)—C₂H₅, —O—C(═O)—C(CH₃)₃, —(CH₂)-pyridinyl, pyridinyl, —O-phenyl, —O-benzyl, phenyl and benzyl, wherein the cyclic part of the respective oxetanyl, (4,5)-dihydroisoxazolyl, thiazolyl, (1,2,5)-thiadiazolyl, thiophenyl, phenethyl, —N—[C(═O)—C₂H₅]-phenyl, —N—[C(═O)—CH₃]-phenyl, —NH-phenyl, —N(CH₃)-phenyl, —N(C₂H₅)-phenyl, —(CH₂)-pyridinyl, pyridinyl, —O-phenyl, —O-benzyl, phenyl and benzyl residues optionally may be substituted with 1, 2, 3, 4 or 5 substituents independently selected from the group consisting of F, Cl, Br, —OH, —CF₃, —SF₅, —CN, —NO₂, methyl, ethyl, n-propyl, isopropyl, n-butyl, sec-butyl, isobutyl, tert-butyl, —O—CH₃, —O—C₂H₅, —O—CH(CH₃)₂, —O—C(CH₃)₃, —O—CF₃, —S—CF₃, phenyl and —O-benzyl.

If one or more of the substituents stand for an aryl residue, this can preferably be selected from the group consisting of phenyl and naphthyl (1-naphthyl and 2-naphthyl).

If one or more of the substituents stand for a heteroaryl residue, this can preferably be selected from the group consisting of tetrazolyl, thiophenyl, furanyl, pyrrolyl, pyrazolyl, pyrazinyl, pyranyl, triazolyl, pyridinyl, imidazolyl, indolyl, isoindolyl, benzo[b]furanyl, benzo[b]thiophenyl, benzoxazolyl, benzisoxazolyl, thiazolyl, oxazolyl, isoxazolyl, pyridazinyl, pyrazinyl, pyrimidinyl, indazolyl, quinoxalinyl, quinolinyl and isoquinolinyl.

Examples of suitable aryl and heteroaryl residues that can be unsubstituted or mono- or polysubstituted and are condensed with a mono- or bicyclic ring system include isoindolyl, indolyl, (1,2,3,4)-tetrahydroquinolinyl, (1,2,3,4)-tetrahydroisoquinolinyl, (2,3)-dihydro-1H-isoindolyl, (1,2,3,4)-tetrahydronaphthyl, (2,3)-dihydro-benzo[1.4]dioxinyl, (2,3)-dihydrothieno[3,4-b][1,4]dioxinyl, benzo[1.3]dioxolyl and (1,4)-benzodioxanyl.

It is particularly preferred that the respective aryl or heteroaryl residues optionally may be substituted with 1, 2, 3, 4 or 5 substituents independently selected from the group consisting of F, Cl, Br, I, —CN, —CF₃, —SF₅, —OH, —O—CH₃, —O—C₂H₅, —O—CH(CH₃)₂, —O—C(CH₃)₃, —NH₂, —NO₂, —O—CF₃, —S—CF₃, —SH, —S—CH₃, —S—C₂H₅, —S—CH(CH₃)₂, —S—C(CH₃)₃, methyl, ethyl, n-propyl, isopropyl, n-butyl, sec-butyl, isobutyl, tert-butyl, n-pentyl, —C(═O)—OH, —C(═O)—O—CH₃, —C(═O)—O—C₂H₅, —C(═O)—O—CH(CH₃)₂, —C(═O)—O—C(CH₃)₃, —NH—CH₃, —NH—C₂H₅, —NH—C(CH₃)₃, —N(CH₃)₂, —N(C₂H₅)₂, —N(CH₃)(C₂H₅), —NH—S(═O)₂—CH₃, —NH—S(═O₂)—C₂H₅, —NH—S(═O)₂—CH(CH₃)₂, —NH—C(═O)—O—CH₃, —NH—C(═O)—O—C₂H₅, —NH—C(═O)—O—C(CH₃)₃, —C(═O)—H, —C(═O)—CH₃, —C(═O)—C₂H₅, —C(═O)—CH(CH₃)₂, —C(═O)—C(CH₃)₃, —C(═O)—NH₂, —C(═O)—NH—CH₃, —C(═O)—NH—C₂H₅, —C(═O)—N(CH₃)₂, —C(═O)—N(C₂H₅)₂, —O-phenyl, —O-benzyl, phenyl and benzyl, wherein the cyclic part of the residues —O-phenyl, —O-benzyl, phenyl and benzyl respectively optionally may be substituted with 1, 2, 3, 4 or 5 substituents independently selected from the group consisting of F, Cl, Br, —OH, —CF₃, —SF₅, —CN, —NO₂, —C₁₋₅-alkyl, —O—C₁₋₅-alkyl, —O—CF₃, —S—CF₃, phenyl and —O-benzyl.

In the sense of the present invention, a mono- or polycyclic ring system is understood to mean mono- or polycyclic hydrocarbon residues that can be saturated or unsaturated and possibly have 1, 2, 3, 4 or 5 heteroatom(s) as ring member(s) independently selected from the group consisting of oxygen, nitrogen and sulfur. Such a mono- or polycyclic ring system can be condensed (anellated), for example, with an aryl residue or a heteroaryl residue.

Where a polycyclic ring system such as a bicyclic ring system, for example, is present, the different rings—respectively independently of one another—can have a different degree of saturation, i.e. be saturated or unsaturated. A polycyclic ring system is preferably a bicyclic ring system. Examples of aryl residues that are condensed with a mono- or polycyclic ring system include (1,3)-benzodioxolyl and (1,4)-benzodioxanyl.

If one or more of the substituents have a mono- or polycyclic ring system, this can preferably be substituted with 1, 2, 3, 4 or 5 substituents independently selected from the group consisting of oxo (═O), thioxo (═S), F, Cl, Br, I, —CN, —CF₃, —SF₅, —OH, —O—CH₃, —O—C₂H₅, —O—CH(CH₃)₂, —O—C(CH₃)₃, —NH₂, —NO₂, —O—CF₃, —S—CF₃, —SH, —S—CH₃, —S—C₂H₅, —S—CH(CH₃)₂, —S—C(CH₃)₃, methyl, ethyl, n-propyl, isopropyl, n-butyl, sec-butyl, isobutyl, tert-butyl, n-pentyl, —C(═O)—OH, —C(═O)—O—CH₃, —C(═O)—O—C₂H₅, —C(═O)—O—CH(CH₃)₂, —C(═O)—O—C(CH₃)₃, —NH—CH₃, —NH—C₂H₅, —NH—C(CH₃)₃, —N(CH₃)₂, —N(C₂H₅)₂, —N(CH₃)(C₂H₅), —NH—C(═O)—O—CH₃, —NH—C(═O)—O—C₂H₅, —NH—C(═O)—O—C(CH₃)₃, —C(═O)—H, —C(═O)—CH₃, —C(═O)—C₂H₅, —C(═O)—CH(CH₃)₂, —C(═O)—C(CH₃)₃, —C(═O)—NH₂, —C(═O)—NH—CH₃, —C(═O) —NH—C₂H₅, —C(═O)—N(CH₃)₂, —C(═O)—N(C₂H₅)₂, —O-phenyl, —O-benzyl, phenyl and benzyl, wherein the cyclic part of the residues —O-phenyl, —O-benzyl, phenyl and benzyl respectively may be optionally substituted with 1, 2, 3, 4 or 5 substituents independently selected from the group consisting of F, Cl, Br, —OH, —CF₃, —SF₅, —CN, —NO₂, —C₁₋₅-alkyl, —O—C₁₋₅-alkyl, —O—CF₃, —S—CF₃, phenyl and —O-benzyl.

If one or more of the substituents has a linear or branched C₁₋₆ alkylene group, this can preferably be selected from the group consisting of —(CH₂)—, —(CH₂)₂—, —C(H)(CH₃)—, —(CH₂)₃—, —(CH₂)₄—, —(CH₂)₅—, —C(H)(C(H)(CH₃)₂)- and —C(C₂H₅)(H)—.

In a further preferred embodiment, at least one compound corresponding to formula (II) can be used for the production of a drug for the treatment of pain:

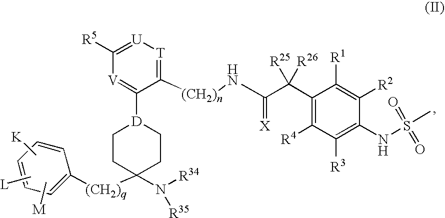

wherein
U, T, V, X, n, R¹, R², R³, R⁴, R⁵, R²⁵ and R²⁶ are defined as above;
D is CH or N;
q is 0, 1, 2 or 3;
K, L and M each independently stand for H, F, Cl, Br, I, —CN, —CF₃, —SF₅, —OH, —O—C₁₋₅-alkyl, —NH₂, —NO₂, —O—CF₃, —S—CF₃, —SH, —S—C₁₋₅-alkyl, —C₁₋₅-alkyl, —C(═O)—OH, —C(═O)—O—C₁₋₅-alkyl, —NH—C₁₋₅-alkyl, —N(C₁₋₅-alkyl)₂, —NH—S(═O)₂—C₁₋₅-alkyl, —NH—C(═O)—O—C₁₋₅-alkyl, —C(═O)—H, —C(═O)—C₁₋₅-alkyl, —C(═O)—NH₂, —C(═O)—NH—C₁₋₅-alkyl, —C(═O)—N—(C₁₋₅-alkyl)₂, —O-phenyl, —O-benzyl, phenyl and benzyl, wherein the cyclic part of the residues —O-phenyl, —O-benzyl, phenyl and benzyl can be respectively substituted with 1, 2, 3, 4 or 5 substituents independently selected from the group consisting of H, F, Cl, Br, I, —CN, —CF₃, —SF₃, —OH, —CF₃, —SF₅, —CN, —NO₂, —C₁₋₅-alkyl, —O—C₁₋₅-alkyl, —O—CF₃, —S—CF₃, phenyl and —O-benzyl; and
R³⁴ and R³⁵ each independently stand for hydrogen or for a linear or branched, saturated or unsaturated aliphatic C₁₋₁₀ residue;
   for an unsaturated or saturated, unsubstituted or mono- or polysubstituted 3-, 4-, 5-, 6-, 7-, 8- or 9-membered cycloaliphatic residue, which is respectively bonded to the basic framework via a carbon atom in the ring of the cycloaliphatic residue and which optionally may be condensed with a saturated or unsaturated, unsubstituted or mono- or polysubstituted mono- or polycyclic ring system and/or can be bonded via a linear or branched, unsubstituted or mono- or polysubstituted C₁₋₆-alkylene group or C₂₋₆-alkenylene group or C₂₋₆-alkinylene group; or
   for an unsubstituted or mono- or polysubstituted 5- to 14-membered aryl or heteroaryl residue, which optionally may be condensed with a saturated or unsaturated, unsubstituted or mono- or polysubstituted mono- or polycyclic ring system and/or may be bonded via a linear or branched, unsubstituted or mono- or polysubstituted C₁₋₆-alkylene group or C₂₋₆-alkenylene group or C₂₋₆-alkinylene group.

In all the preferred embodiments corresponding to formulas (I) and (II) specified below D, K, L, M, p and q are defined as above.

Compounds with a dual mode of action corresponding to the foregoing formulas (I) and (II) are preferred, wherein:
X stands for O, S or N—C≡N;
W stands for —CN, —NR³⁴R³⁵, —C(═O)—R³⁶ or —C(═O)—OR³⁷;
n stands for 0, 1, 2, 3 or 4;
R¹, R², R³ and R⁴ each independently stand for H; F; Cl; Br; I; —SF₅; —NO₂; —CN; —CF₃; —NH₂; —OH; —SH; —C(═O)—NH₂; —S(═O)₂—NH₂; —C(═O)—NH—OH; —C(═O)—OH; —C(═O)—H; —S(═O)₂—OH; —NHR¹¹; —NR¹²R¹³; —OR¹⁴; —SR¹⁵; —C(═O)—NHR¹⁶; —C(═O)—NR¹⁷R¹⁸; —S(═O)₂—NHR¹⁹; —S(═O)₂—NR²⁰R²¹; —C(═O)—OR²²; —C(═O)—R²³; —S(═O)—R²⁴; —S(═O)₂—²⁴ or for an alkyl residue selected from the group consisting of methyl, ethyl, n-propyl, isopropyl, tert-butyl, n-butyl, sec-butyl, isobutyl, n-pentyl, n-hexyl and n-heptyl;
R⁵ stands for H; F; Cl; Br; I; —SF₅; —NO₂; —CF₃; —CF₂Cl; —CN; —NH₂; —OH; —SH; —C(═O)—NH₂; —S(═O)₂—NH₂; —C(═O)—NH—OH; —C(═O)—OH; —C(═O)—H; —S(═O)₂—OH; —NHR¹¹; —NR¹²R¹³; —OR¹⁴; —SR¹⁵; —C(═O)—NHR¹⁶; —C(═O)—NR¹⁷R¹⁸; —S(═O)₂—NHR¹⁹; —S(═O)₂—NR²⁰R²¹; —C(═O)—OR²²; —C(═O)—R²³; —S(═O)—R²⁴; —S(═O)₂—R²⁴;
   for an alkyl residue selected from the group consisting of methyl, ethyl, n-propyl, isopropyl, tert-butyl, n-butyl, sec-butyl, isobutyl, n-pentyl, n-hexyl and n-heptyl; or
   for a residue selected from the group consisting of cyclopropyl, cyclobutyl, cyclopentyl, cyclohexyl, cycloheptyl, cyclopentenyl, cyclohexenyl, cycloheptenyl, imidazolidinyl, tetrahydrofuranyl, tetrahydrothiophenyl, pyrrolidinyl, piperidinyl, morpholinyl, piperazinyl, azepanyl, diazepanyl, azocanyl and thiomorpholinyl, which is respectively bonded to the basic framework via a carbon atom of the rings of the aforementioned residues and optionally may be substituted with 1, 2, 3, 4 or 5 substituents independently selected from the group consisting of —CH₂—O—CH₃, —CH₂—O—CH₂—CH₃, oxo (═O), thioxo (═S), F, Cl, Br, I, —CN, —CF₃, —SF₅, —OH, —O—CH₃, —O—C₂H₅, —O—CH(CH₃)₂, —O—C(CH₃)₃, —NH₂, —NO₂, —O—CF₃, —S—CF₃, —SH, —S—CH₃, —S—C₂H₅, —S—CH(CH₃)₂, —S—C(CH₃)₃, methyl, ethyl, n-propyl, isopropyl, n-butyl, sec-butyl, isobutyl, tert-butyl, n-pentyl, —C(═O)—CH₃, —C(═O)—C₂H₅, —C(═O)—CH(CH₃)₂, —C(═O)—C(CH₃)₃, —C(═O)—OH, —C(═O)—O—CH₃, —C(═O)—O—C₂H₅, —C(═O)—O—CH(CH₃)₂, —C(═O)—O—C(CH₃)₃, —NH—CH₃, —NH—C₂H₅, —NH—C(CH₃)₃, —N(CH₃)₂, —N(C₂H₅)₂, —N(CH₃)(C₂H₅), —NH-phenyl, —N(CH₃)-phenyl, —N(C₂H₅)-phenyl, —N(C₂H₅)-phenyl, —O—CH₂—CH₂—CH₂—CH₃, piperidinyl, pyrrolidinyl, —O—C(═O)—CH₃, —O—C(═O)—C₂H₅, —O—C(═O)—C(CH₃)₃, —(CH₂)-pyridinyl, pyridinyl, —O-phenyl, —O-benzyl, phenyl and benzyl, wherein the cyclic part of the —NH-phenyl, —N(CH₃)-phenyl, —N(C₂H₅)-phenyl, —(CH₂)-pyridinyl, pyridinyl, —O-phenyl, —O-benzyl, phenyl and benzyl residues optionally may be substituted with 1, 2, 3, 4 or 5 substituents independently selected from the group consisting of F, Cl, Br, —OH, —CF₃, —SF₅, —CN, —NO₂, methyl, ethyl, n-propyl, isopropyl, n-butyl, sec-butyl, isobutyl, tert-butyl, —O—CH₃, —O—C₂H₅, —O—CH(CH₃)₂, —O—C(CH₃)₃, —O—CF₃, —S—CF₃, phenyl and —O-benzyl;
T stands for C—R⁶ and U for C—R⁷ and V for N;
or
T stands for C—R⁶ and U for N and V for C—R⁹;
or
T stands for N and U for C—R⁷ and V for C—R⁹;
or
T stands for N and U for N and V for C—R⁹;
or
T stands for N and U for C—R⁷ and V for N;
or
T stands for C—R⁶ and U for N and V for N;
or
T stands for C—R⁶ and U for C—R⁷ and V for C—R⁹;
R⁶ and R⁷ each independently stand for H; F; Cl; Br; I; —SF₅; —NO₂; —CF₃; —CF₂Cl; —CN; —NH₂; —OH; —SH; —C(═O)—NH₂; —S(═O)₂—NH₂; —C(═O)—OH; —C(═O)—H; —S(═O)₂—OH; —NHR¹¹; —NR¹²R¹³; —OR¹⁴; —SR¹⁵; —C(═O)—NHR¹⁶; —C(═O)—NR¹⁷R¹⁸; —S(═O)₂—NHR¹⁹; —S(═O)₂—NR²⁰R²¹; —C(═O)—OR²²; —C(═O)—R²³, S(═O)—R²⁴; —S(═O)₂—R²⁴; for an alkyl residue selected from the group consisting of methyl, ethyl, n-propyl, isopropyl, tert-butyl, n-butyl, sec-butyl, isobutyl, n-pentyl, n-hexyl and n-heptyl or for a phenyl group, which optionally may be bonded via a —(CH═CH)—, —C≡C—, —(CH₂)—, —(CH₂)₂— or —(CH₂)₃ group and/or optionally may be substituted with 1, 2, 3, 4 or 5 substituents independently selected from the group consisting of F, Cl, Br, I, —CN, —CF₃, —SF₅, —OH, —O—CH₃, —O—C₂H₅, —O—CH(CH₃)₂, —O—C(CH₃)₃, —NH₂, —NO₂, —O—CF₃, —S—CF₃, —SH, —S—CH₃, —S—C₂H₅, —S—CH(CH₃)₂, —S—C(CH₃)₃, methyl, ethyl, n-propyl, isopropyl, n-butyl, sec-butyl, isobutyl, tert-butyl and n-pentyl;
R⁹ stands for H; F; Cl; Br; I; —SF₅; —NO₂; —CF₃; —CF₂Cl; —CN; —NH₂; —OH; —SH; —C(═O)—NH₂; —S(═O)₂—NH₂; —C(═O)—NH—OH; —C(═O)—OH; —C(═O)—H; —S(═O)₂—OH; —NHR¹¹; —NR¹²R¹³; —OR¹⁴; —SR¹⁵; —C(═O)—NHR¹⁶; —C(═O)—NR¹⁷R¹⁸; —S(═O)₂—NHR¹⁹; —S(═O)₂—NR²⁰R²¹; —C(═O)—OR²²; —C(═O)—R²³; —S(═O)—R²⁴; —S(═O)₂—R²⁴ or for an alkyl residue selected from the group consisting of methyl, ethyl, n-propyl, isopropyl, tert-butyl, n-butyl, sec-butyl, isobutyl, n-pentyl, n-hexyl and n-heptyl;
R¹¹, R¹², R¹³, R¹⁴, R¹⁵, R¹⁶, R¹⁷, R¹⁸, R¹⁹, R²⁰, R²¹, R²², R²³ and R²⁴ each independently stand for an alkyl residue selected from the group consisting of methyl, ethyl, n-propyl, isopropyl, tert-butyl, n-butyl, sec-butyl, isobutyl, n-pentyl, n-hexyl, n-heptyl, 3-pentyl, 4-heptyl, n-octyl, n-nonyl, 5-nonyl, (2,6)-dimethyl-hept-4-yl, 3-methyl-butyl, n-hexyl, (3,3)-dimethylbutyl, ethenyl, propenyl, 2-butenyl, 3-butenyl, 2-pentenyl and 3-pentenyl;
   for a residue selected from the group consisting of 2,3-dihydro-1H-indenyl, cyclopropyl, oxetanyl, cyclobutyl, cyclopentyl, cyclohexyl, cycloheptyl, cyclopentenyl, cyclohexenyl, cycloheptenyl, imidazolidinyl, tetrahydrofuranyl, tetrahydrothiophenyl, pyrrolidinyl, piperidinyl, morpholinyl, piperazinyl, azepanyl, diazepanyl, azocanyl and thiomorpholinyl, which optionally may be bonded via a —CH₂—O—, —CH₂—CH₂—O—, —CH₂—CH₂—O—CH₂—, —CH₂—CH(CH₃)—O—CH₂, —(CH₂)—, —(CH₂)₂— or —(CH₂)₃ group and/or optionally may be substituted with 1, 2, 3, 4 or 5 substituents independently selected from the group consisting of —CH₂—O—CH₃, —CH₂—O—CH₂—CH₃, oxo (═O), thioxo (═S), F, Cl, Br, I, —CN, —CF₃, —SF₅, —OH, —O—CH₃, —O—C₂H₅, —O—CH(CH₃)₂, —O—C(CH₃)₃, —NH₂, —NO₂, —O—CF₃, —S—CF₃, —SH, —S—CH₃, —S—C₂H₅, —S—CH(CH₃)₂, —S—C(CH₃)₃, methyl, ethyl, n-propyl, -isopropyl, n-butyl, sec-butyl, isobutyl, tert-butyl, n-pentyl, —C(═O)—CH₃, —C(═O)—C₂H₅, —C(═O)—CH(CH₃)₂, —C(═O)—C(CH₃)₃, —C(═O)—OH, —C(═O)—O—CH₃, —C(═O)—O—C₂H₅, —C(═O)—O—CH(CH₃)₂, —C(═O)—O—C(CH₃)₃, —NH—CH₃, —NH—C₂H₅, —NH—C(CH₃)₃, —N(CH₃)₂, —N(C₂H₅)₂, —N(CH₃)(C₂H₅), —NH-phenyl, —N(CH₃)-phenyl, —N(C₂H₅)-phenyl, —N(C₂H₅)-phenyl, —O—CH₂—CH₂—CH₂—CH₃, piperidinyl, pyrrolidinyl, —O—C(═O)—CH₃, —O—C(═O)—C₂H₅, —O—C(═O)—C(CH₃)₃, —(CH₂)-pyridinyl, pyridinyl, —O-phenyl, —O-benzyl, phenyl and benzyl, wherein the cyclic part of the —NH-phenyl, —N(CH₃)-phenyl, —N(C₂H₅)-phenyl, —(CH₂)-pyridinyl, pyridinyl, —O-phenyl, —O-benzyl, phenyl and benzyl residues optionally may be substituted with 1, 2, 3, 4 or 5 substituents independently selected from the group consisting of F, Cl, Br, —OH, —CF₃, —SF₅, —CN, —NO₂, methyl, ethyl, n-propyl, isopropyl, n-butyl, sec-butyl, isobutyl, tert-butyl, —O—CH₃, —O—C₂H₅, —O—CH(CH₃)₂, —O—C(CH₃)₃, —O—CF₃, —S—CF₃, phenyl and —O-benzyl; or
   for a residue selected from the group consisting of phenyl, naphthyl, (1,3)-benzodioxolyl, (1,4)-benzodioxanyl, thiophenyl, furanyl, pyrrolyl, pyrazolyl, pyrazinyl, pyranyl, triazolyl, pyridinyl, imidazolyl, indolyl, isoindolyl, benzo[b]furanyl, benzo[b]thiophenyl, benzoxazolyl, benzisoxazolyl, thiazolyl, oxazolyl, isoxazolyl, pyridazinyl, pyrazinyl, pyrimidinyl, indazolyl, quinoxalinyl, quinolinyl and isoquinolinyl, wherein the residue optionally may be bonded via a —(CH₂)—, —(CH₂)₂— or —(CH₂)₃ group and/or optionally may be substituted with 1, 2, 3, 4 or 5 substituents independently selected from the group consisting of F, Cl, Br, I, —CN, —CF₃, —SF₅, —OH, —O—CH₃, —O—C₂H₅, —O—CH(CH₃)₂, —O—C(CH₃)₃, —NH₂, —NO₂, —O—CF₃, —S—CF₃, —SH, —S—CH₃, —S—C₂H₅, —S—CH(CH₃)₂, —S—C(CH₃)₃, methyl, ethyl, n-propyl, isopropyl, n-butyl, sec-butyl, isobutyl, tert-butyl, n-pentyl, —C(═O)—OH, —C(═O)—O—CH₃, —C(═O)—O—C₂H₅, —C(═O)—O—CH(CH₃)₂, —C(═O)—O—C(CH₃)₃, —NH—CH₃, —NH—C₂H₅, —NH—C(CH₃)₃, —N(CH₃)₂, —N(C₂H₅)₂, —N(CH₃)(C₂H₅), —NH—C(═O)—O—CH₃, —NH—C(═O)—O—C₂H₅, —NH—C(═O)—O—C(CH₃)₃, —C(═O)—H, —C(═O)—CH₃, —C(═O)—C₂H₅, —C(═O)—CH(CH₃)₂, —C(═O)—C(CH₃)₃, —C(═O)—NH₂, —C(═O)—NH—CH₃, —C(═O)—NH—C₂H₅, —C(═O)—N(CH₃)₂, —C(═O)—N(C₂H₅)₂, —O-phenyl, —O-benzyl, phenyl and benzyl, wherein the cyclic part of the —O-phenyl, —O-benzyl, phenyl and benzyl residues optionally may be substituted with 1, 2, 3, 4 or 5 substituents independently selected from the group consisting of F, Cl, Br, —OH, —CF₃, —SF₅, —CN, —NO₂, —C₁₋₅-alkyl, —O—C₁₋₅-alkyl, —O—CF₃, —S—CF₃, phenyl and —O-benzyl; or
R¹² and R¹³ together with the nitrogen atom connecting them form a residue selected from the group consisting of 3-aza-bicyclo[3.1.1]heptyl, 6-aza-spiro[2.5]octyl, 3-aza-bicyclo[3.2.1]octyl, 6-aza-bicyclo[3.3.1]heptyl, 8-aza-bicyclo[3.2.1]octyl, 1-oxa-2,8-diaza-spiro[4.5]dec-2-enyl, azocanyl, isoindolyl, indolyl, (1,2,3,6)-tetrahydropyridinyl, (4,5,6,7)-tetrahydroisoxazolo[5,4-c]pyridinyl, pyrrolidinyl, piperidinyl, (1,3,4,5)-tetrahydropyrido[4,3-b]indolyl, (3,4)-dihydro-1H-isoquinolinyl, (1,3,4,9)-tetrahydro-[b]-carbolinyl, imidazolidinyl, (1,3)-thiazolidinyl, piperazinyl, morpholinyl, azepanyl, diazepanyl and thiomorpholinyl, which can respectively be optionally substituted with 1, 2, 3, 4 or 5 substituents independently selected from the group consisting of —CH₂—O—CH₂-oxetanyl, —CH₂—OH, —CH₂—CH₂—OH, ═CH₂, —O—CH₂-oxetanyl, (4,5)-dihydroisoxazolyl, thiazolyl, (1,2,5)-thiadiazolyl, thiophenyl, phenethyl, —CN, —CH₂—N(CH₃)₂, —CH₂—N(C₂H₅)₂, —CH₂—NH—CH₃, —CH₂—NH—C₂H₅, —N—[C(═O)—C₂H₅]-phenyl, —N—[C(═O)—CH₃]-phenyl, —CH₂—O—CH₃, —CH₂—O—CH₂—CH₃, oxo (═O), thioxo (═S), F, Cl, Br, I, —CN, —CF₃, —SF₅, —OH, —O—CH₃, —O—C₂H₅, —O—CH(CH₃)₂, —O—C(CH₃)₃, —NH₂, —NO₂, —O—CF₃, —S—CF₃, —SH, —S—CH₃, —S—C₂H₅, —S—CH(CH₃)₂, —S—C(CH₃)₃, methyl, ethyl, n-propyl, isopropyl, n-butyl, sec-butyl, isobutyl, tert-butyl, n-pentyl, —C(═O)—CH₃, —C(═O)—C₂H₅, —C(═O)—CH(CH₃)₂, —C(═O)—C(CH₃)₃, —C(═O)—OH, —C(═O)—O—CH₃, —C(═O)—O—C₂H₅, —C(═O)—O—CH(CH₃)₂, —C(═O)—O—C(CH₃)₃, —NH—CH₃, —NH—C₂H₅, —NH—C(CH₃)₃, —N(CH₃)₂, —N(C₂H₅)₂, —N(CH₃)(C₂H₅), —NH-phenyl, —N(CH₃)-phenyl, —N(C₂Hs)-phenyl, —N(C₂H₅)— phenyl, —O—CH₂—CH₂—CH₂—CH₃, piperidinyl, pyrrolidinyl, cyclohexyl, cyclopentyl, —O—C(═O)—CH₃, —O—C(═O)—C₂H₅, —O—C(═O)—C(CH₃)₃, —(CH₂)-pyridinyl, pyridinyl, —O-phenyl, —O-benzyl, phenyl and benzyl, wherein the cyclic part of the oxetanyl, (4,5)-dihydroisoxazolyl, thiazolyl, (1,2,5)-thiadiazolyl, thiophenyl, phenethyl, —N—[C(═O)—C₂H₅]-phenyl, —N—[C(═O)—CH₃]-phenyl, —NH-phenyl, —N(CH₃)-phenyl, —N(C₂H₅)-phenyl, —(CH₂)-pyridinyl, pyridinyl, —O-phenyl, —O-benzyl, phenyl and benzyl residues optionally may be substituted with 1, 2, 3, 4 or 5 substituents independently selected from the group consisting of F, Cl, Br, —OH, —CF₃, —SF₅, —CN, —NO₂, methyl, ethyl, n-propyl, isopropyl, n-butyl, sec-butyl, isobutyl, tert-butyl, —O—CH₃, —O—C₂H₅, —O—CH(CH₃)₂, —O—C(CH₃)₃, —O—CF₃, —S—CF₃, phenyl and —O-benzyl;
R²⁵ and R²⁶ each independently stand for hydrogen;
   for an alkyl residue selected from the group consisting of methyl, ethyl, n-propyl, isopropyl, tert-butyl, n-butyl, sec-butyl, isobutyl, n-pentyl, n-hexyl and n-heptyl;
   for a residue selected from the group consisting of phenyl, naphthyl, (1,3)-benzodioxolyl, (1,4)-benzodioxanyl, thiophenyl, furanyl, pyrrolyl, pyrazolyl, pyrazinyl, pyranyl, triazolyl, pyridinyl, imidazolyl, indolyl, isoindolyl, benzo[b]furanyl, benzo[b]thiophenyl, benzoxazolyl, benzisoxazolyl, thiazolyl, oxazolyl, isoxazolyl, pyridazinyl, pyrazinyl, pyrimidinyl, indazolyl, quinoxalinyl, quinolinyl and isoquinolinyl, wherein the residue optionally may be bonded via a —(CH₂)—, —(CH₂)₂— or —(CH₂)₃ group and/or optionally may be substituted with 1, 2, 3, 4 or 5 substituents independently selected from the group consisting of F, Cl, Br, I, —CN, —CF₃, —SF₅, —OH, —O—CH₃, —O—C₂H₅, —O—CH(CH₃)₂, —O—C(CH₃)₃, —NH₂, —NO₂, —O—CF₃, —S—CF₃, —SH, —S—CH₃, —S—C₂H₅, —S—CH(CH₃)₂, —S—C(CH₃)₃, methyl, ethyl, n-propyl, isopropyl, n-butyl, sec-butyl, isobutyl, tert-butyl and n-pentyl; or
   for a residue selected from the group consisting of cyclopropyl, cyclobutyl, cyclopentyl, cyclohexyl, cyclopentenyl and cyclohexenyl;
with the proviso that R²⁵ and R²⁶ do not simultaneously stand for hydrogen;
or
R²⁵ and R²⁶ together with the carbon atom connecting them form a residue selected from the group consisting of cyclopropyl, cyclobutyl, cyclopentyl, cyclohexyl, cycloheptyl, cyclopentenyl and cyclohexenyl;
   wherein unless otherwise specified, the aforementioned alkyl, alkenyl and alkinyl residues each may optionally be substituted with 1, 2, 3, 4, 5, 6, 7, 8 or 9 substituents independently selected from the group consisting of —C(═O)—O—CH₃, —C(═O)—O—C₂H₅, —C(═O)—O—C(CH₃)₃, —O—C(═O)—CH₃, —O—C(═O)—C₂H₅, —O—C(═O)—CH(CH₃)₂, —O—C(═O)—C(CH₃)₃, —O-phenyl, phenyl, F, Cl, Br, I, —CN, —NO₂, —OH, —NH₂, —SH, —O—CH₃, —O—C₂H₅, —O—CH(CH₃)₂, —O—C(CH₃)₃, —S—CH₃, —S—C₂H₅, —S—CH(CH₃)₂, —S—C(CH₃)₃, —NH—CH₃, —NH—C₂H₅, —NH—C(CH₃)₃, —N(CH₃)₂, —N(C₂H₅)₂, —N(CH₃)(C₂H₅), —OCF₃ and —SCF₃;
and
R³⁴, R³⁵, R³⁶ and R³⁷ each independently stand for hydrogen or for a linear or branched, saturated or unsaturated aliphatic C₁₋₁₀ residue;
   for an unsaturated or saturated, unsubstituted or mono- or polysubstituted 3-, 4-, 5-, 6-, 7-, 8- or 9-membered cycloaliphatic residue which is respectively bonded to the basic framework via a carbon atom in the ring of the cycloaliphatic residue and which optionally may be condensed with a saturated or unsaturated, unsubstituted or mono- or polysubstituted mono- or polycyclic ring system and/or may be bonded via a linear or branched, unsubstituted or mono- or polysubstituted C₁₋₆-alkylene group or C₂₋₆-alkenylene group or C₂₋₆-alkinylene group; or
   for an unsubstituted or mono- or polysubstituted 5- to 14-membered aryl or heteroaryl residue, which optionally may be condensed with a saturated or unsaturated, unsubstituted or mono- or polysubstituted mono- or polycyclic ring system and/or may be bonded via a linear or branched, unsubstituted or mono- or polysubstituted C₁₋₆-alkylene group or C₂₋₆-alkenylene group or C₂₋₆-alkinylene group;
      in the form of one of their individual stereoisomers, in particular enantiomers or diastereomers, their racemates or in the form of a mixture of stereoisomers, in particular enantiomers and/or diastereomers, in any desired mixture ratio, or respectively in the form of corresponding salts, or respectively in the form of corresponding solvates.

Also preferred are compounds with a dual mode of action corresponding to the foregoing formulas (I) and (II), wherein
X stands for O, S or N—C≡N;
W stands for —CN or —NR³⁴R³⁵;
n stands for 0, 1, 2, 3 or 4;
R¹, R², R³ and R⁴ each independently stand for H; F; Cl; Br; I; —SF₅; —NO₂; —CN; —NH₂; —OH; —SH; —C(═O)—NH₂; —S(═O)₂—NH₂; —C(═O)—NH—OH; —C(═O)—OH; —C(═O)—H; —S(═O)₂—OH; —NHR¹¹; —NR¹²R¹³; —OR¹⁴; —SR¹⁵; —S(═O)—R²⁴; —S(═O)₂—R²⁴ or for a residue selected from the group consisting of methyl, —CF₃, —CCl₃, —CBr₃, —CHF₂, —CH₂F, —CF₂Cl, —CCl₂F, ethyl, —CF₂—CH₃, —CH₂—CF₃, —C₂F₅, —CH₂—CCl₃, —CH₂—CBr₃, —CHF—CF₂Cl, —CF₂—CF₂Cl, —CFCl—CF₂Cl, n-propyl, —CF₂—CF₂—CF₃, —CF(CF₃)₂, isopropyl, n-butyl, sec-butyl, isobutyl and tert-butyl;
R⁵ stands for F; Cl; Br; I; —SF₅; —OR¹⁴; —SR¹⁵; —S(═O)—R²⁴; —S(═O)₂—R²⁴;
   for a residue selected from the group consisting of methyl, —CF₃, —CCl₃, —CBr₃, —CHF₂, —CH₂F, —CF₂Cl, —CCl₂F, —CH₂—CN, —CH₂—O—CH₃, —CH₂—O—CF₃, —CH₂—SF₃, ethyl, —CF₂—CH₃, —CH₂—CF₃, —C₂F₅, —CH₂—CCl₃, —CH₂—CBr₃, —CHF—CF₂Cl, —CF₂—CF₂Cl, —CFCl—CF₂Cl, —CH₂—CH₂—CN, n-propyl, —CF₂—CF₂—CF₃, —CF(CF₃)₂, isopropyl, —CH₂—CH₂—CH₂—CN, —CH₂—O—CH₂—CH₃, —CH₂—CH₂—SF₃, —CH₂—CH₂—OCF₃, —CH(CH₃)(O—CH₃), —CH(CH₃)(S—CH₃), n-butyl, —CF₂—CF₂—CF₂—CF₃, —CH₂—CH₂—CH₂—CH₂—CN, n-butyl, sec-butyl, isobutyl, —C(CH₃)₂(CH₂OH), and tert-butyl; or
   for a residue selected from the group consisting of cyclopropyl, cyclobutyl, cyclopentyl, cyclohexyl, cyclopentenyl and cyclohexenyl, which optionally may be substituted with 1, 2, 3, 4 or 5 substituents independently selected from the group consisting of oxo (═O), thioxo (═S), —OH, —O—CH₃, —O—C₂H₅, —O—CH(CH₃)₂, —O—C(CH₃)₃, —O—CF₃, —S—CF₃, —SH, —S—CH₃, —S—C₂H₅, —S—CH(CH₃)₂, —S—C(CH₃)₃, methyl, ethyl, n-propyl, isopropyl, n-butyl, sec-butyl, isobutyl, tert-butyl and n-pentyl;
T stands for C—R⁶ and U for C—R⁷ and V for N;
or
T stands for C—R⁶ and U for N and V for C—R⁹;
or
T stands for N and U for C—R⁷ and V for C—R⁹;
or
T stands for N and U for N and V for C—R⁹;
or
T stands for N and U for C—R⁷ and V for N;
or
T stands for C—R⁶ and U for N and V for N;
or
T stands for C—R⁶ and U for C—R⁷ and V for C—R⁹;
R⁶ and R⁷ each independently stand for H; F; Cl; Br; I; —SF₅; —NO₂; —CN; —NH₂; —OH; —SH; —C(═O)—NH₂; —S(═O)₂—NH₂; —C(═O)—NH—OH; —C(═O)—OH; —C(═O)—H; —S(═O)₂—OH; —NHR¹¹; —NR¹²R¹³; —OR¹⁴; —SR¹⁵; —C(═O)—OR²²; —S(═O)—R²⁴; —S(═O)₂—R²⁴; for a residue selected from the group consisting of —CH₂—OH, methyl, —CF₃, —CCl₃, —CBr₃, —CHF₂, —CH₂F, —CF₂Cl, —CCl₂F, ethyl, —CF₂—CH₃, —CH₂—CF₃, —C₂F₅, —CH₂—CCl₃, —CH₂—CBr₃, —CHF—CF₂CJ, —CF₂—CF₂Cl, —CFCl—CF₂Cl, n-propyl, —CF₂—CF₂—CF₃, —CF(CF₃)₂, isopropyl, n-butyl, sec-butyl, isobutyl and tert-butyl or for a phenyl residue, which optionally may be substituted with 1, 2, 3, 4 or 5 substituents independently selected from the group consisting of F, Cl, Br, I, —CN, —CF₃, —SF₅, —OH, —O—CH₃, —O—C₂H₅, —O—CH(CH₃)₂, —O—C(CH₃)₃, —NH₂, —NO₂, —O—CF₃, —S—CF₃, —SH, —S—CH₃, —S—C₂H₅, —S—CH(CH₃)₂, —S—C(CH₃)₃, methyl, ethyl, n-propyl, isopropyl, n-butyl, sec-butyl, isobutyl, tert-butyl and n-pentyl;
R⁹ stands for H; F; Cl; Br; I; —SF₅; —NO₂; —CN; —NH₂; —OH; —SH; —C(═O)—NH₂; —S(═O)₂—NH₂; —C(═O)—NH—OH; —C(═O)—OH; —C(═O)—H; —S(═O)₂—OH; —NHR¹¹; —NR¹²R¹³; —OR¹⁴; —SR¹⁵; —S(═O)—R²⁴; —S(═O)₂—R²⁴ or for a residue selected from the group consisting of methyl, —CF₃, —CCl₃, —CBr₃, —CHF₂, —CH₂F, —CF₂Cl, —CCl₂F, ethyl, —CF₂—CH₃, —CH₂—CF₃, —C₂F₅, —CH₂—CCl₃, —CH₂—CBr₃, —CHF—CF₂Cl, —CF₂—CF₂Cl, —CFCl—CF₂Cl, n-propyl, —CF₂—CF₂—CF₃, —CF(CF₃)₂, isopropyl, n-butyl, sec-butyl, isobutyl and tert-butyl;
R¹¹, R¹², R¹³, R¹⁴, R¹⁵, R²² and R²⁴ each independently stand for a residue selected from the group consisting of methyl, —CF₃, —CCl₃, —CBr₃, —CHF₂, —CH₂F, —CF₂Cl, —CCl₂F, —CH₂—CN, —CH₂—O—CH₃, —CH₂—O—CF₃, —CH₂—SF₃, ethyl, —CF₂—CH₃, —CH₂—CF₃, —C₂F₅, —CH₂—CCl₃, —CH₂—CBr₃, —CHF—CF₂Cl, —CF₂—CF₂Cl, —CFCl—CF₂Cl, —CH₂—CH₂—CN, n-propyl, —CF₂—CF₂—CF₃, —CF(CF₃)₂, isopropyl, —CH₂—CH₂—CH₂—CN, —CH₂—O—CH₂—CH₃, —CH₂—CH₂—SF₃, —CH₂—CH₂—OCF₃, —CH(CH₃)(O—CH₃), —CH(CH₃)(S—CH₃), n-butyl, —CF₂—CF₂—CF₂—CF₃, —CH₂—CH₂—CH₂—CH₂—CN, sec-butyl, isobutyl, tert-butyl, n-pentyl, 3-pentyl, n-heptyl, 4-heptyl, n-octyl, n-nonyl, 5-nonyl, (2,6)-dimethyl-hept-4-yl, 3-methyl-butyl, n-hexyl, (3,3)-dimethylbutyl, —CH₂—CH₂—O—CH₃, —CH₂—CH₂—O—C₂H₅, —CH₂—CH₂—O-phenyl, —CH₂—CH₂—CH₂—O—CH₃, ethenyl, propenyl, 2-butenyl, 3-butenyl, 2-pentenyl and 3-pentenyl;
   for a residue selected from the group consisting of 2,3-dihydro-1H-indenyl, cyclopropyl, oxetanyl, cyclobutyl, cyclopentyl, cyclohexyl, imidazolidinyl, tetrahydrofuranyl, tetrahydrothiophenyl, pyrrolidinyl, piperidinyl, morpholinyl, piperazinyl, azepanyl, diazepanyl, azocanyl and thiomorpholinyl, which optionally may be bonded via a —CH₂—O—, —CH₂—CH₂—O—, —CH₂—CH₂—O—CH₂—, —CH₂—CH(CH₃)—O—CH₂—, —(CH₂)—, —(CH₂)₂— or —(CH₂)₃ group and/or can be respectively optionally substituted with 1, 2, 3, 4 or 5 substituents independently selected from the group consisting of oxo (═O), thioxo (═S), —OH, —O—CH₃, —O—C₂H₅, —O—CH(CH₃)₂, —O—C(CH₃)₃, —O—CF₃, —S—CF₃, —SH, —S—CH₃, —S—C₂H₅, —S—CH(CH₃)₂, —S—C(CH₃)₃, methyl, ethyl, n-propyl, isopropyl, n-butyl, sec-butyl, isobutyl, tert-butyl, n-pentyl, —C(═O)—CH₃, —C(═O)—C₂H₅, —C(═O)—CH(CH₃)₂, —C(═O)—C(CH₃)₃, —C(═O)—OH, —C(═O)—O—CH₃, —C(═O)—O—C₂H₅, —C(═O)—O—CH(CH₃)₂ and —C(═O)—O—C(CH₃)₃; or
   for a residue selected from the group consisting of phenyl, naphthyl, thiophenyl, furanyl, pyrrolyl, pyrazolyl, pyrazinyl, pyranyl, triazolyl, pyridinyl, imidazolyl, thiazolyl, oxazolyl and isoxazolyl, wherein the residue can respectively be bonded via a —(CH₂)—, —(CH₂)₂— or —(CH₂)₃ group and/or can be respectively optionally substituted with 1, 2, 3, 4 or 5 substituents independently selected from the group consisting of F, Cl, Br, I, —CN, —CF₃, —SF₅, —OH, —O—CH₃, —O—C₂H₅, —O—CH(CH₃)₂, —O—C(CH₃)₃, —NH₂, —NO₂, —O—CF₃, —S—CF₃, —SH, —S—CH₃, —S—C₂H₅, —S—CH(CH₃)₂, —S—C(CH₃)₃, methyl, ethyl, n-propyl, isopropyl, n-butyl, sec-butyl, isobutyl, tert-butyl, n-pentyl, —C(═O)—OH, —C(═O)—O—CH₃, —C(═O)—O—C₂H₅, —C(═O)—O—CH(CH₃)₂, —C(═O)—O—C(CH₃)₃, —NH—CH₃, —NH—C₂H₅, —NH—C(CH₃)₃, —N(CH₃)₂, —N(C₂H₅)₂, —N(CH₃)(C₂H₅), —NH—C(═O)—O—CH₃, —NH—C(═O)—O—C₂H₅, —NH—C(═O)—O—C(CH₃)₃, —C(═O)—H, —C(═O)—CH₃, —C(═O)—C₂H₅, —C(═O)—CH(CH₃)₂, —C(═O)—C(CH₃)₃, —C(═O)—NH₂, —C(═O)—NH—CH₃, —C(═O)—NH—C₂H₅, —C(═O)—N(CH₃)₂, —C(═O)—N(C₂H₅)₂, —O-phenyl, —O-benzyl, phenyl and benzyl; or
R¹² and R¹³ together with the nitrogen atom connecting them form a residue selected from the group consisting of 3-aza-bicyclo[3.1.1]heptyl 3-aza-bicyclo[3.1.1]heptyl, 6-aza-spiro[2.5]octyl, 3-aza-bicyclo[3.2.1]octyl, 6-aza-bicyclo[3.3.1]heptyl, 8-aza-bicyclo[3.2.1]octyl, 1-oxa-2,8-diaza-spiro[4.5]dec-2-enyl, azocanyl, isoindolyl, indolyl, (1,2,3,6)-tetrahydropyridinyl, (4,5,6,7)-tetrahydroisoxazolo-[5,4-c]pyridinyl, pyrrolidinyl, piperidinyl, piperazinyl, morpholinyl, azepanyl, diazepanyl and thiomorpholinyl, which can be respectively optionally substituted with 1, 2, 3, 4 or 5 substituents independently selected —CH₂—O—CH₂-oxetanyl, —CH₂—OH, —CH₂—CH₂—OH, ═CH₂, —O—CH₂-oxetanyl, (4,5)-dihydroisoxazolyl, thiazolyl, (1,2,5)-thiadiazolyl, thiophenyl, phenethyl, —CN, —CH₂—N(CH₃)₂, —CH₂—N(C₂H₅)₂, —CH₂—NH—CH₃, —CH₂—NH—C₂H₅, —N—[C(═O)—C₂H₅]-phenyl, —N—[C(═O)—CH₃]-phenyl, —CH₂—O—CH₃, —CH₂—O—CH₂—CH₃, oxo (═O), thioxo (═S), F, Cl, Br, I, —CN, —CF₃, —SF₅, —OH, —O—CH₃, —O—C₂H₅, —O—CH(CH₃)₂, —O—C(CH₃)₃, —NH₂, —NO₂, —O—CF₃, —S—CF₃, —SH, —S—CH₃, —S—C₂H₅, —S—CH(CH₃)₂, —S—C(CH₃)₃, methyl, ethyl, n-propyl, isopropyl, n-butyl, sec-butyl, isobutyl, tert-butyl, n-pentyl, —C(═O)—CH₃, —C(═O)—C₂H₅, —C(═O)—CH(CH₃)₂, —C(═O)—C(CH₃)₃, —C(═O)—OH, —C(═O)—O—CH₃, —C(═O)—O—C₂H₅, —C(═O)—O—CH(CH₃)₂, —C(═O)—O—C(CH₃)₃, —NH—CH₃, —NH—C₂H₅, —NH—C(CH₃)₃, —N(CH₃)₂, —N(C₂H₅)₂, —N(CH₃)(C₂H₅), —NH-phenyl, —N(CH₃)-phenyl, —N(C₂H₅)-phenyl, —N(C₂H₅)-phenyl, —O—CH₂—CH₂—CH₂—CH₃, cyclohexyl, cyclopentyl, piperidinyl, pyrrolidinyl, —O—C(═O)—CH₃, —O—C(═O)—C₂H₅, —O—C(═O)—C(CH₃)₃, —(CH₂)-pyridinyl, pyridinyl, —O-phenyl, —O-benzyl, phenyl and benzyl, wherein the cyclic part of the residues oxetanyl, (4,5)-dihydroisoxazolyl, thiazolyl, (1,2,5)-thiadiazolyl, thiophenyl, phenethyl, —N—[C(═O)—C₂H₅]-phenyl, —N—[C(═O)—CH₃]-phenyl, —NH-phenyl, —N(CH₃)-phenyl, —N(C₂H₅)-phenyl, —(CH₂)-pyridinyl, pyridinyl, —O-phenyl, —O-benzyl, phenyl and benzyl can be respectively substituted with 1, 2, 3, 4 or 5 substituents independently selected from the group consisting of —CF₃, F, Cl, Br, —OH, —CF₃, —SF₅, —CN, —NO₂, methyl, ethyl, n-propyl, isopropyl, n-butyl, sec-butyl, isobutyl, tert-butyl, —O—CH₃, —O—C₂H₅, —O—CH(CH₃)₂, —O—C(CH₃)₃, —O—CF₃, —S—CF₃, phenyl and —O-benzyl;
R²⁵ and R²⁶ each independently stand for hydrogen;
   for an alkyl residue selected from the group consisting of —CH₂—OH, —CH₂—CH₂—OH, —CH₂—CH₂—CH₂—OH, —CH₂—CH₂—CH₂—CH₂—OH, isopropyl, n-butyl, sec-butyl, isobutyl, methyl, ethyl and n-propyl;
   for a residue selected from the group consisting of phenyl, naphthyl, thiophenyl, furanyl, pyrrolyl, pyrazolyl, pyrazinyl, pyranyl, triazolyl, pyridinyl, imidazolyl, indolyl, isoindolyl, thiazolyl, oxazolyl, isoxazolyl, pyridazinyl, pyrazinyl, pyrimidinyl, indazolyl, quinoxalinyl, quinolinyl and isoquinolinyl, which optionally can be bonded via a —(CH₂)—, —(CH₂)₂— or —(CH₂)₃ group and/or optionally may be substituted with 1, 2, 3, 4 or 5 substituents independently selected from the group consisting of F, Cl, Br, I, —CN, —CF₃, —SF₅, —OH, —O—CH₃, —O—C₂H₅, —O—CH(CH₃)₂, —O—C(CH₃)₃, —NH₂, —NO₂, —O—CF₃, —S—CF₃, —SH, —S—CH₃, —S—C₂H₅, —S—CH(CH₃)₂, —S—C(CH₃)₃, methyl, ethyl, n-propyl, isopropyl, n-butyl, sec-butyl, isobutyl, tert-butyl and n-pentyl; or
   for a residue selected from the group consisting of cyclopropyl, cyclobutyl, cyclopentyl and cyclohexyl;
with the proviso that R²⁵ and R²⁶ do not simultaneously stand for hydrogen;
or
R²⁵ and R²⁶ together with the carbon atom connecting them form a residue selected from the group consisting of cyclopropyl, cyclobutyl, cyclopentyl, cyclohexyl and cycloheptyl; and
R³⁴ and R³⁵ each independently stand for hydrogen or for a linear or branched, saturated or unsaturated aliphatic C₁₋₁₀ residue;
   for an unsaturated or saturated, unsubstituted or mono- or polysubstituted 3-, 4-, 5-, 6-, 7-, 8- or 9-membered cycloaliphatic residue which is respectively bonded to the basic framework via a carbon atom in the ring of the cycloaliphatic residue and which optionally may be condensed with a saturated or unsaturated, unsubstituted or mono- or polysubstituted mono- or polycyclic ring system and/or may be bonded via a linear or branched, unsubstituted or mono- or polysubstituted C₁₋₆-alkylene group or C₂₋₆-alkenylene group or C₂₋₆-alkinylene group;
      in the form of one of their individual stereoisomers, in particular enantiomers or diastereomers, their racemates or in the form of a mixture of stereoisomers, in particular enantiomers and/or diastereomers, in any desired mixture ratio, or respectively in the form of corresponding salts, or respectively in the form of corresponding solvates.

Additionally preferred are compounds with a dual mode of action corresponding to the foregoing formulas (I) and (II), wherein
X stands for O, S or N—C≡N;
W stands for —NR³⁴R³⁵;
n stands for 0, 1 or 2;
R¹, R³ and R⁴ each independently stand for H; F; Cl; Br; or for a residue selected from the group consisting of methyl, —CF₃, —CCl₃, —CBr₃, —CHF₂, —CH₂F, —CF₂Cl, —CCl₂F, ethyl, —CF₂—CH₃, —CH₂—CF₃, —C₂F₅, —CH₂—CCl₃, —CH₂—CBr₃, —CHF—CF₂Cl, —CF₂—CF₂Cl and —CFCl—CF₂Cl;
R² stands for F; Cl; Br; I or for a residue selected from the group consisting of methyl, —CF₃, —CCl₃, —CBr₃, —CHF₂, —CH₂F, —CF₂Cl, —CCl₂F, ethyl, —CF₂—CH₃, —CH₂—CF₃, —C₂F₅, —CH₂—CCl₃, —CH₂—CBr₃, —CHF—CF₂Cl, —CF₂—CF₂Cl, —CFCl—CF₂Cl, n-propyl, —CF₂—CF₂—CF₃, —CF(CF₃)₂, isopropyl, sec-butyl, isobutyl, tert-butyl, —O—CH₃, —O—CF₃, —O—CCl₃, —O—CBr₃, —O—CHF₂, —O—CH₂F, —O—CF₂Cl, —O—CCl₂F, —O—C₂H₅, —O—CF₂—CH₃, —O—CH₂—CF₃, —O—C₂F₅, —O—CH₂—CCl₃, —O—CH₂—CBr₃, —O—CHF—CF₂Cl, —O—CF₂—CF₂Cl, —O—CFCl—CF₂Cl, —O—CH₂—CH₂—CH₃, —O—CF₂—CF₂—CF₃, —O—CF(CF₃)₂, —O—CH(CH₃)₂, —O—C(CH₃)₃, —S—CH₃, —S—CF₃, —S—CCl₃, —S—CBr₃, —S—CHF₂, —S—CH₂F, —S—CF₂Cl, —S—CCl₂F, —S—C₂H₅, —S—CF₂—CH₃, —S—CH₂—CF₃, —S—C₂F₅, —S—CH₂—CCl₃, —S—CH₂—CBr₃, —S—CHF—CF₂Cl, —S—CF₂—CF₂Cl, —S—CFCl—CF₂Cl, —S—CH₂—CH₂—CH₃, —S—CF₂—CF₂—CF₃, —S—CF(CF₃)₂, —S—CH(CH₃)₂ and —S—C(CH₃)₃;
R⁵ stands for F; Cl; Br; I; —SF₅; or
   for a residue selected from the group consisting of methyl, ethyl, —CF₃, —CCl₃, —CBr₃, —CHF₂, —CH₂F, —CF₂Cl, —CCl₂F, —CF₂—CH₃, —CH₂—CF₃, —C₂F₅, —CH₂—CCl₃, —CH₂—CBr₃, —CHF—CF₂Cl, —CF₂—CF₂Cl, —CFCl—CF₂Cl, —CF₂—CF₂—CF₃, —CF(CF₃)₂, sec-butyl, isobutyl, —C(CH₃)₂(CH₂OH), tert-butyl, —O—CF₃, —O—CCl₃, —O—CBr₃, —O—CHF₂, —O—CH₂F, —O—CF₂Cl, —O—CCl₂F, —O—CF₂—CH₃, —O—CH₂—CF₃, —O—C₂F₅, —O—CH₂—CCl₃, —O—CH₂—CBr₃, —O—CHF—CF₂Cl, —O—CF₂—CF₂Cl, —O—CFCl—CF₂Cl, —O—CF₂—CF₂—CF₃, —O—CF(CF₃)₂, —O—CH(CH₃)₂, —O—C(CH₃)₃, —S—CF₃, —S—CCl₃, —S—CBr₃, —S—CHF₂, —S—CH₂F, —S—CF₂Cl, —S—CCl₂F, —S—CF₂—CH₃, —S—CH₂—CF₃, —S—C₂F₅, —S—CH₂—CCl₃, —S—CH₂—CBr₃, —S—CHF—CF₂Cl, —S—CF₂—CF₂Cl, —S—CFCl—CF₂Cl, —S—CF₂—CF₂—CF₃, —S—CF(CF₃)₂, —S—CH(CH₃)₂, —S—C(CH₃)₃, —S(═O)₂—CF₃, —S(═O)₂—CCl₃, —S(═O)₂—CBr₃, —S(═O)₂—CHF₂, —S(═O)₂—CH₂F, —S(═O)₂—CF₂Cl, —S(═O)₂—CCl₂F, —S(═O)₂—CF₂—CH₃, —S(═O)₂—CH₂—CF₃, —S(═O)₂—C₂F₅, —S(═O)₂—CH₂—CCl₃, —S(═O)₂—CH₂—CBr₃, —S(═O)₂—CHF—CF₂Cl, —S(═O)₂—CF₂—CF₂Cl, —S(═O)₂—CFCl—CF₂Cl, —S(═O)₂—CF₂—CF₂—CF₃, —S(═O)₂—CF(CF₃)₂, —S(═O)₂—CH(CH₃)₂ and —S(═O)₂—C(CH₃)₃; or
   for a residue selected from the group consisting of cyclopropyl, cyclobutyl, cyclopentyl, cyclohexyl, cyclopentenyl and cyclohexenyl;
T stands for C—R⁶ and U for C—R⁷ and V for N;
or
T stands for C—R⁶ and U for N and V for C—R⁹;
or
T stands for N and U for C—R⁷ and V for C—R⁹;
or
T stands for N and U for N and V for C—R⁹;
or
T stands for N and U for C—R⁷ and V for N;
or
T stands for C—R⁶ and U for N and V for N;
or
T stands for C—R⁶ and U for C—R⁷ and V for C—R⁹;
R⁶ and R⁷ each independently stand for H; F; Cl; Br; I; —NO₂; —CN; —C(═O)—OCH₃; —C(═O)—OC₂H₅; for a residue selected from the group consisting of —CH₂—OH, methyl, —CF₃, —CCl₃, —CBr₃, —CHF₂, —CH₂F, —CF₂Cl, —CCl₂F, ethyl, n-propyl, isopropyl, sec-butyl, isobutyl and tert-butyl or for a phenyl residue, which can possibly be substituted with 1, 2, 3, 4 or 5 substituents independently selected from the group consisting of F, Cl, Br, I, —CN, —CF₃, —SF₅, —OH, —O—CH₃, —O—C₂H₅, —O—CH(CH₃)₂, —O—C(CH₃)₃, —NH₂, —NO₂, —O—CF₃, —S—CF₃, —SH, —S—CH₃, —S—C₂H₅, —S—CH(CH₃)₂, —S—C(CH₃)₃, methyl, ethyl, n-propyl, isopropyl, n-butyl, sec-butyl, isobutyl, tert-butyl and n-pentyl;
R⁹ stands for H; F; Cl; Br; I; —NO₂; —CN; or for a residue selected from the group consisting of methyl, —CF₃, —CCl₃, —CBr₃, —CHF₂, —CH₂F, —CF₂Cl, —CCl₂F, ethyl, n-propyl, —CF₂—CF₂—CF₃, —CF(CF₃)₂, isopropyl, sec-butyl, isobutyl and tert-butyl;
R²⁵ and R²⁶ each independently stand for hydrogen;
   for an alkyl residue selected from the group consisting of —CH₂—OH, —CH₂—CH₂—OH, —CH₂—CH₂—CH₂—OH, —CH₂—CH₂—CH₂—CH₂—OH, isopropyl, n-butyl, sec-butyl, isobutyl, methyl, ethyl and n-propyl or for a residue selected from the group consisting of phenyl, benzyl, phenethyl, cyclopropyl, cyclobutyl, cyclopentyl and cyclohexyl;
with the proviso that R²⁵ and R²⁶ do not simultaneously stand for hydrogen;
or
R²⁵ and R²⁶ together with the carbon atom connecting them form a residue selected from the group consisting of cyclopropyl, cyclobutyl, cyclopentyl, cyclohexyl and cycloheptyl; and
R³⁴ and R³⁵ each independently stand for hydrogen or for a linear or branched, saturated or unsaturated aliphatic C₁₀ residue;
   for an unsaturated or saturated, unsubstituted or mono- or polysubstituted 3-, 4-, 5-, 6-, 7-, 8- or 9-membered cycloaliphatic residue which is respectively bonded to the basic framework via a carbon atom in the ring of the cycloaliphatic residue and which optionally may be condensed with a saturated or unsaturated, unsubstituted or mono- or polysubstituted mono- or polycyclic ring system and/or may be bonded via a linear or branched, unsubstituted or mono- or polysubstituted C₁₋₆-alkylene group or C₂₋₆-alkenylene group or C₂₋₆-alkinylene group;
      in the form of one of their individual stereoisomers, in particular enantiomers or diastereomers, their racemates or in the form of a mixture of stereoisomers, in particular enantiomers and/or diastereomers, in any desired mixture ratio, or respectively in the form of corresponding salts, or respectively in the form of corresponding solvates.

Also preferred are compounds corresponding to the foregoing formulas (I) and (II),

wherein
X stands for O or S;
W stands for —NR³⁴R³⁵;
n stands for 0, 1 or 2;
R¹, R³ and R⁴ respectively stand for H;
R² stands for F; Cl; Br; I or for a residue selected from the group consisting of methyl, —CF₃, —CCl₃, —CBr₃, —CHF₂, —CH₂F, —O—CH₃, —O—CF₃, —O—CCl₃, —O—CBr₃, —O—CHF₂, —O—CH₂F, —S—CH₃, —S—CF₃, —S—CCl₃, —S—CBr₃, —S—CHF₂, —S—CH₂F, —S—CF₂Cl and —S—CCl₂F;
R⁵ stands for F; Cl; Br; I; —SF₅;
   for a residue selected from the group consisting of methyl, ethyl, —CF₃, —CCl₃, —CBr₃, —CHF₂, —CH₂F, —CF₂Cl, —CCl₂F, —C(CH₃)₂(CH₂OH), tert-butyl, —O—CF₃, —O—CCl₃, —O—CBr₃, —O—CHF₂, —O—CH₂F, —O—CF₂Cl, —O—CCl₂F, —O—CF₂—CH₃, —S—CF₃, —S—CCl₃, —S—CBr₃, —S—CHF₂, —S—CH₂F, —S—CF₂Cl, —S—CCl₂F, —S—CF₂—CH₃, —S(═O)₂—CF₃, —S(═O)₂—CCl₃, —S(═O)₂—CBr₃, —S(═O)₂—CHF₂, —S(═O)₂—CH₂F and —S(═O)₂—CF₂Cl; or
   for a residue selected from the group consisting of cyclopropyl, cyclobutyl, cyclopentyl, cyclohexyl, cyclopentenyl and cyclohexenyl;
T stands for C—R⁶ and U for C—R⁷ and V for N;
or
T stands for C—R⁶ and U for N and V for C—R⁹;
or
T stands for N and U for C—R⁷ and V for C—R⁹;
or
T stands for N and U for N and V for C—R⁹;
or
T stands for N and U for C—R⁷ and V for N;
or
T stands for C—R⁶ and U for N and V for N;
or
T stands for C—R⁶ and U for C—R⁷ and V for C—R⁹;
R⁶ and R⁷ each independently stand for —CF₃; phenyl; —C(═O)—OCH₃; —C(═O)—OC₂H₅; methyl; —CH₂—OH; H; F; Cl; Br or I;
R⁹ stands for —CF₃; H; F; Cl; Br or I;
R²⁵ and R²⁶ each independently stand for hydrogen;
   for an alkyl residue selected from the group consisting of —CH₂—OH, —CH₂—CH₂—OH, —CH₂—CH₂—CH₂—OH, —CH₂—CH₂—CH₂—CH₂—OH, isopropyl, n-butyl, sec-butyl, isobutyl, methyl, ethyl and n-propyl or for a residue selected from the group consisting of phenyl, benzyl, phenethyl, cyclopropyl, cyclobutyl, cyclopentyl and cyclohexyl;
with the proviso that R²⁵ and R²⁶ do not simultaneously stand for hydrogen;
or
R²⁵ and R²⁶ together with the carbon atom connecting them form a cyclic residue selected from the group consisting of cyclopropyl, cyclobutyl, cyclopentyl, cyclohexyl and cycloheptyl; and
R³⁴ and R³⁵ each independently stand for hydrogen or for a linear or branched, saturated or unsaturated aliphatic C₁₋₁₀ residue;
   in the form of one of their individual stereoisomers, in particular enantiomers or diastereomers, their racemates or in the form of a mixture of stereoisomers, in particular enantiomers and/or diastereomers, in any desired mixture ratio, or respectively in the form of corresponding salts, or respectively in the form of corresponding solvates.

Additionally preferred are compounds with dual mode of action corresponding to the foregoing formulas (I) and (II), wherein
X stands for O;
W stands for —NR³⁴R³⁵;
n stands for 1;
R¹, R³ and R⁴ respectively stand for H;
R² stands for methyl; —O—CH₃; F; Cl; Br or I;
R⁵ stands for a residue selected from the group consisting of methyl, ethyl, —CF₃, —CCl₃, —CBr₃, —CHF₂, —CH₂F, —CF₂Cl, —CCl₂F, —C(CH₃)₂(CH₂OH), tert-butyl, —O—CF₃, —O—CCl₃, —O—CBr₃, —O—CHF₂, —O—CH₂F, —S—CF₃, —S—CCl₃, —S—CBr₃, —S—CHF₂ and —S—CH₂F; or
   for a residue selected from the group consisting of cyclopropyl, cyclobutyl, cyclopentyl, cyclohexyl, cyclopentenyl and cyclohexenyl;
T stands for CH and U for CH and V for N;
or
T stands for CH and U for N and V for CH;
or
T stands for N and U for CH and V for CH;
or
T stands for N and U for N and V for CH;
or
T stands for N and U for CH and V for N;
or
T stands for CH and U for N and V for N;
or
T stands for CH and U for CH and V for CH;
R²⁵ stands for an alkyl residue selected from the group consisting of —CH₂—OH, —CH₂—CH₂—OH, methyl, ethyl and n-propyl or for a residue selected from the group consisting of benzyl, phenyl, phenethyl, cyclopropyl, cyclobutyl, cyclopentyl and cyclohexyl;
R²⁶ stands for hydrogen or for a residue selected from the group consisting of methyl, ethyl and n-propyl; or
R²⁵ and R²⁶ together with the carbon atom connecting them form a residue selected from the group consisting of cyclopropyl, cyclobutyl, cyclopentyl and cyclohexyl; and
R³⁴ and R³⁵ each independently stand for hydrogen or for a linear or branched, saturated or unsaturated aliphatic C₁₋₁₀ residue;
   in the form of one of their individual stereoisomers, in particular enantiomers or diastereomers, their racemates or in the form of a mixture of stereoisomers, in particular enantiomers and/or diastereomers, in any desired mixture ratio, or respectively in the form of corresponding salts, or respectively in the form of corresponding solvates.

In a further preferred embodiment at least one compound corresponding to formula (III) can be used for production of a pharmaceutical composition for the treatment of pain:

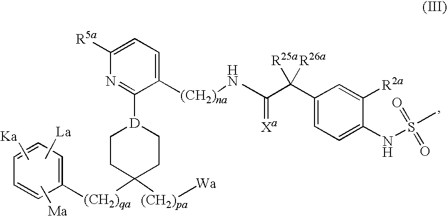

wherein
na stands for 0, 1 or 2;
R^{2a} stands for F; Cl; Br; I or for a residue selected from the group consisting of methyl, —CF₃, —CCl₃, —CBr₃, —CHF₂, —CH₂F, —O—CH₃, —O—CF₃, —O—CCl₃, —O—CBr₃, —O—CHF₂, —O—CH₂F, —S—CH₃, —S—CF₃, —S—CCl₃, —S—CBr₃, —S—CHF₂, —S—CH₂F, —S—CF₂Cl and —S—CCl₂F;
R^{5a} stands for F; Cl; Br; I; —SF₅;
   for a residue selected from the group consisting of methyl, ethyl, —CF₃, —CCl₃, —CBr₃, —CHF₂, —CH₂F, —CF₂Cl, —CCl₂F, —C(CH₃)₂—(CH₂OH), tert-butyl, —O—CF₃, —O—CCl₃, —O—CBr₃, —O—CHF₂, —O—CH₂F, —O—CF₂Cl, —O—CCl₂F, —O—CF₂—CH₃, —S—CF₃, —S—CCl₃, —S—CBr₃, —S—CHF₂, —S—CH₂F, —S—CF₂Cl, —S—CCl₂F, —S—CF₂—CH₃, —S(═O)₂—CF₃, —S(═O)₂—CCl₃, —S(═O)₂—CBr₃, —S(═O)₂—CHF₂, —S(═O)₂—CH₂F and —S(═O)₂—CF₂Cl; or
   for a residue selected from the group consisting of cyclopropyl, cyclobutyl, cyclopentyl, cyclohexyl, cyclopentenyl and cyclohexenyl;
R^{25a} and R^{26a} each independently stand for hydrogen, or
   for an alkyl residue selected from the group consisting of —CH₂—OH, —CH₂—CH₂—OH, —CH₂—CH₂—CH₂—OH, —CH₂—CH₂—CH₂—CH₂—OH, isopropyl, n-butyl, sec-butyl, isobutyl, methyl, ethyl and n-propyl; with the proviso that R^{25a} and R^{26a} are not simultaneously hydrogen; or
R^{25a} and R^{26a} together with the carbon atom connecting them form a residue selected from the group consisting of cyclopropyl, cyclobutyl, cyclopentyl, cyclohexyl and cycloheptyl;
X^{a} stands for O or S;
D stands for CH or N;
pa stands for 0;
qa stands for 0, 1 or 2;
Ka, La and Ma each independently stand for H, —CF₃, —OH, —O—CH₃, —O—C₂H₅, F, Cl, Br, methyl, ethyl, isopropyl, n-propyl, n-butyl, tert-butyl or sec-butyl;
Wa stands for —N^{34a}R^{35a}, —CN, —C(═O)—R^{36a} or C(═O)—OR^{37a}; and
R^{34a}, R^{35a}, R^{36a} and R^{37a} each independently stand for H or for a residue selected from the group consisting of methyl, ethyl, n-propyl, isopropyl, n-butyl, sec-butyl and isobutyl;
   in the form of one of its individual stereoisomers, in particular enantiomers or diastereomers, its racemates or in the form of a mixture of stereoisomers, in particular enantiomers and/or diastereomers, in any desired mixture ratio, or respectively in the form of corresponding salts, or respectively in the form of corresponding solvates.

In a likewise preferred embodiment at least one compound corresponding to formula (IV) can be used for the production of a drug for the treatment of pain:

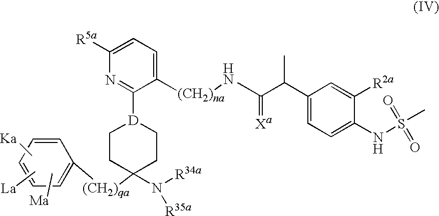

wherein
na, R^{2a}, R^{5a} and X^{a} are defined as above;
D stands for CH or N;
qa stands for 0, 1 or 2;
Ka, La and Ma each independently stand for H, —CF₃, —OH, —O—CH₃, —O—C₂H₅, F, Cl, Br, methyl, ethyl, isopropyl, n-propyl, n-butyl, tert-butyl or sec-butyl; and
R^{34a} and R^{35a} each independently stand for H or for a residue selected from the group consisting of methyl, ethyl, n-propyl, isopropyl, n-butyl, sec-butyl and isobutyl;
   in the form of one of its individual stereoisomers, in particular enantiomers or diastereomers, its racemates or in the form of a mixture of stereoisomers, in particular enantiomers and/or diastereomers, in any desired mixture ratio, or respectively in the form of corresponding salts, or respectively in the form of corresponding solvates.

In a further preferred embodiment at least one compound corresponding to formula (V) can be used for the production of a drug for the treatment of pain:

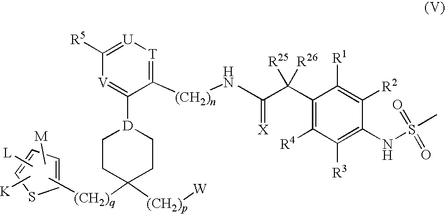

wherein
D is CH or N;
W stands for —CN, —NR³⁴R³⁵, —C(═O)—R³⁶ or —C(═O)—OR³⁷;
X stands for O, S or N—C≡N;
n stands for 0, 1, 2, 3 or 4;
p is 0, 1, 2 or 3;
q is 0, 1, 2 or 3;
T stands for C—R⁶ and U for C—R⁷ and V for N;
or
T stands for C—R⁶ and U for N and V for C—R⁹;
or
T stands for N and U for C—R⁷ and V for C—R⁹;
or
T stands for N and U for N and V for C—R⁹;
or
T stands for N and U for C—R⁷ and V for N;
or
T stands for C—R⁶ and U for N and V for N;
or
T stands for C—R⁶ and U for C—R⁷ and V for C—R⁹;
R¹, R², R³ and R⁴ each independently stand for H; F; Cl; Br; I; —SF₅; —NO₂; —CF₃; —CN; —NH₂; —OH; —SH; —C(═O)—NH₂; —S(═O)₂—NH₂; —C(═O)—NH—OH; —C(═O)—OH; —C(═O)—H; —S(═O)₂—OH; —NHR¹¹; —NR¹²R¹³; —OR¹⁴; —SR¹⁵; —C(═O)—NHR¹⁶; —C(═O)—NR¹⁷R¹⁸; —S(═O)₂—NHR¹⁹; —S(═O)₂—NR²⁰R²¹; —C(═O)—OR²²; —C(═O)—R²³; —S(═O)—R²⁴; —S(═O)₂—R²⁴ or for a linear or branched, saturated or unsaturated, unsubstituted or mono- or polysubstituted aliphatic C₁₋₁₀ residue;
R⁵ stands for H; F; Cl; Br; I; —SF₅; —NO₂; —CF₃; —CF₂Cl; —CN; —NH₂; —OH; —SH; —C(═O)—NH₂; —S(═O)₂—NH₂; —C(═O)—NH—OH; —C(═O)—OH; —C(═O)—H; —S(═O)₂—OH; —NHR¹¹; —NR¹²R¹³; —OR¹⁴; —SR¹⁵; —C(═O)—NHR¹⁶; —C(═O)—NR¹⁷R¹⁸; —S(═O)₂—NHR¹⁹; —S(═O)₂—NR²⁰R²¹; —C(═O)—OR²²; —C(═O)—R²³, —S(═O)₂—R²⁴; —S(═O)—R²⁴;
   for a linear or branched, saturated or unsaturated, unsubstituted or mono- or polysubstituted aliphatic C₁₋₁₀ residue; or
   for an unsaturated or saturated, unsubstituted or mono- or polysubstituted 3-, 4-, 5-, 6-, 7-, 8- or 9-membered cycloaliphatic residue, which optionally may contain one or more heteroatoms as ring member and is respectively bonded to the basic framework via a carbon atom in the ring of the cycloaliphatic residue;
R⁶ and R⁷ each independently stand for H; F; Cl; Br; I; —SF₅; —NO₂; —CF₃; —CF₂Cl; —CN; —NH₂; —OH; —SH; —C(═O)—NH₂; —S(═O)₂—NH₂; —C(═O)—NH—OH; —C(═O)—OH; —C(═O)—H; —S(═O)₂—OH; —NHR¹¹; —NR¹²R¹³; —OR¹⁴; —SR¹⁵; —C(═O)—NHR¹⁶; —C(═O)—NR¹⁷R¹⁸; —S(═O)₂—NHR¹⁹; —S(═O)₂—NR²⁰R²¹; —C(═O)—OR²²; —C(═O)—R²³, —S(═O)—R²⁴; —S(═O)₂—R²⁴;
   for a linear or branched, saturated or unsaturated, unsubstituted or mono- or polysubstituted aliphatic C₁₋₁₀ residue; or
   for an unsubstituted or mono- or polysubstituted 6- or 10-membered aryl residue, which can be bonded via a linear or branched, unsubstituted or mono- or polysubstituted C₁₋₆-alkylene group or C₂₋₆-alkenylene group or C₂₋₆-alkinylene group;
R⁹ stands for H; F; Cl; Br; I; —SF₅; —NO₂; —CF₃; —CF₂Cl; —CN; —NH₂; —OH; —SH; —C(═O)—NH₂; —S(═O)₂—NH₂; —C(═O)—NH—OH; —C(═O)—OH; —C(═O)—H; —S(═O)₂—OH; —NHR¹¹; —NR¹²R¹³; —OR¹⁴; —SR¹⁵; —C(═O)—NHR¹⁶; —C(═O)—NR¹⁷R¹⁸; —S(═O)₂—NHR¹⁹; —S(═O)₂—NR²⁰R²¹; —C(═O)—OR²²; —C(═O)—R²³; —S(═O)—R²⁴; —S(═O)₂—R²⁴ or for a linear or branched, saturated or unsaturated, unsubstituted or mono- or polysubstituted aliphatic C₁₋₁₀ residue;
R¹¹, R¹², R¹³, R¹⁴, R¹⁵, R¹⁶, R¹⁷, R¹⁸, R¹⁹, R²⁰, R²¹, R²², R²³ and R²⁴ each independently stand for a linear or branched, saturated or unsaturated, unsubstituted or mono- or polysubstituted aliphatic C₁₋₁₀ residue;
   for an unsaturated or saturated, unsubstituted or mono- or polysubstituted 3-, 4-, 5-, 6-, 7-, 8- or 9-membered cycloaliphatic residue, which optionally may contain one or more heteroatoms as ring members and optionally may be condensed with a saturated or unsaturated, unsubstituted or mono- or polysubstituted mono- or polycyclic ring system and/or may be bonded via a linear or branched, unsubstituted or mono- or polysubstituted C₁₋₆-alkylene group or 2- to 6-membered heteroalkylene group; or
   for an unsubstituted or mono- or polysubstituted 5- to 14-membered aryl or heteroaryl residue, which optionally may be condensed with a saturated or unsaturated, unsubstituted or mono- or polysubstituted mono- or polycyclic ring system and/or can be bonded via a linear or branched, unsubstituted or mono- or polysubstituted C₁₋₆-alkylene group or 2- to 6-membered heteroalkylene group; or
R¹² and R¹³ together with the nitrogen atom connecting them form a saturated or unsaturated, unsubstituted or mono- or polysubstituted 4-, 5-, 6-, 7-, 8- or 9-membered heterocycloaliphatic residue, which optionally may contain one or more further heteroatoms as ring members and which optionally may be condensed with a saturated or unsaturated, unsubstituted or mono- or polysubstituted mono- or polycyclic ring system;
K, L and M each independently stand for H, F, Cl, Br, I, —CN, —CF₃, —SF₅, —OH, —O—C₁₋₅-alkyl, —NH₂, —NO₂, —O—CF₃, —S—CF₃, —SH, —S—C₁₋₅-alkyl, —C₁₋₅-alkyl, —C(═O)—OH, —C(═O)—O—C₁₋₅ alkyl, —NH—C₁₋₅-alkyl, —N(C₁₋₅-alkyl)₂, —NH—S(═O)₂—C₁₋₅-alkyl, —NH—C(═O)—O—C₁₋₅-alkyl, —C(═O)—H, —C(═O)—C₁₋₅-alkyl, —C(═O)—NH₂, —C(═O)—NH—C₁₋₅-alkyl, —C(═O)—N—(C₁₋₅-alkyl)₂, —O-phenyl, —O-benzyl, phenyl and benzyl, wherein the cyclic part of the residues —O-phenyl, —O-benzyl, phenyl and benzyl can be respectively substituted with 1, 2, 3, 4 or 5 substituents independently selected from the group consisting of F, Cl, Br, —OH, —CF₃, —SF₅, —CN, —NO₂, —C₁₋₅-alkyl, —O—C₁₋₅-alkyl, —O—CF₃, —S—CF₃, phenyl and —O-benzyl;
R²⁵ and R²⁶ each independently stand for hydrogen;
   for a linear or branched, saturated or unsaturated, unsubstituted or mono- or polysubstituted aliphatic Cl₁₀ residue;
   for an unsubstituted or mono- or polysubstituted 5- to 14-membered aryl or heteroaryl residue, which optionally may be condensed with a saturated or unsaturated, unsubstituted or mono- or polysubstituted mono- or polycyclic ring system and/or can be bonded via a linear or branched, unsubstituted or mono- or polysubstituted C₁₋₆-alkylene group or C₂₋₆-alkenylene group or C₂₋₆-alkinylene group; or
   for an unsaturated or saturated, unsubstituted or mono- or polysubstituted 3-, 4-, 5-, 6-, 7-, 8- or 9-membered cycloaliphatic residue, which optionally may contain one or more heteroatoms as ring members;
with the proviso that R²⁵ and R²⁶ do not simultaneously stand for hydrogen;
or
R²⁵ and R²⁶ together with the carbon atom connecting them form a saturated or unsaturated, unsubstituted or mono- or polysubstituted 3-, 4-, 5- or 6-membered cycloaliphatic residue; and
R³⁴, R³⁵, R³⁶ and R³⁷ each independently stand for hydrogen, or for a linear or branched, saturated or unsaturated aliphatic C₁₋₁₀ residue;
   for an unsaturated or saturated, unsubstituted or mono- or polysubstituted 3-, 4-, 5-, 6-, 7-, 8- or 9-membered cycloaliphatic residue, which is respectively bonded to the basic framework via a carbon atom in the ring of the cycloaliphatic residue and which optionally may be condensed with a saturated or unsaturated, unsubstituted or mono- or polysubstituted mono- or polycyclic ring system and/or may be bonded via a linear or branched, unsubstituted or mono- or polysubstituted C₁₋₆-alkylene group or C₂₋₆-alkenylene group or C₂₋₆-alkinylene group; or
   for an unsubstituted or mono- or polysubstituted 5- to 14-membered aryl or heteroaryl residue, which optionally may be condensed with a saturated or unsaturated, unsubstituted or mono- or polysubstituted mono- or polycyclic ring system and/or may be bonded via a linear or branched, unsubstituted or mono- or polysubstituted C₁₋₆-alkylene group or C₂₋₆-alkenylene group or C₂₋₆-alkinylene group;
      in the form of one of its individual stereoisomers, in particular enantiomers or diastereomers, its racemates or in the form of a mixture of stereoisomers, in particular enantiomers and/or diastereomers, in any desired mixture ratio, or respectively in the form of corresponding salts, or respectively in the form of corresponding solvates.

In all the above-specified embodiments corresponding to formula (V) D, K, L, M, p and q are defined as above.

Additionally preferred are compounds with a dual mode of action corresponding to formula (V), wherein
X stands for O, S or N—C≡N;
W stands for —CN, —NR³⁴R³⁵, —C(═O)—R³⁶ or —C(═O)—OR³⁷;
n stands for 0, 1, 2, 3 or 4;
R¹, R², R³ and R⁴ each independently stand for H; F; Cl; Br; I; —SF₅; —NO₂; —CN; —CF₃; —NH₂; —OH; —SH; —C(═O)—NH₂; —S(═O)₂—NH₂; —C(═O)—NH—OH; —C(═O)—OH; —C(═O)—H; —S(O)₂—OH; —NHR¹¹; —NR¹²R¹³; —OR¹⁴; —SR⁵; —C(═O)—NHR¹⁶; —C(═O)—NR¹⁷R¹⁸; —S(═O)₂—NHR¹⁹; —S(═O)₂—NR²⁰R²¹; —C(═O)—OR²²; —C(═O)—R²³; S(═O)—R²⁴—S(═O)₂—R²⁴ or for an alkyl residue selected from the group consisting of methyl, ethyl, n-propyl, isopropyl, tert-butyl, n-butyl, sec-butyl, isobutyl, n-pentyl, n-hexyl and n-heptyl;
R⁵ stands for H; F; Cl; Br; I; —SF₅; —NO₂; —CF₃; —CF₂Cl; —CN; —NH₂; —OH; —SH; —C(═O)—NH₂; —S(═O)₂—NH₂; —C(═O)—NH—OH; —C(═O)—OH; —C(═O)—H; —S(═O)₂—OH; —NHR¹¹; —NR¹²R¹³; —OR¹⁴; —SR¹⁵; —C(═O)—NHR¹⁶; —C(═O)—NR¹⁷R¹⁸; —S(═O)₂—NHR¹⁹; —S(═O)₂—NR²⁰R²¹; —C(O)—OR²²; —C(═O)—R²³; —S(═O)—R²⁴; —S(═O)₂—R²⁴;
   for an alkyl residue selected from the group consisting of methyl, ethyl, n-propyl, isopropyl, tert-butyl, n-butyl, sec-butyl, isobutyl, n-pentyl, n-hexyl and n-heptyl; or
   for a residue selected from the group consisting of cyclopropyl, cyclobutyl, cyclopentyl, cyclohexyl, cycloheptyl, cyclopentenyl, cyclohexenyl, cycloheptenyl, imidazolidinyl, tetrahydrofuranyl, tetrahydrothiophenyl, pyrrolidinyl, piperidinyl, morpholinyl, piperazinyl, azepanyl, diazepanyl, azocanyl and thiomorpholinyl, which is respectively bonded to the basis framework via a carbon atom of the rings of the aforementioned residues and can be respectively optionally substituted with 1, 2, 3, 4 or 5 substituents independently selected from the group consisting of —CH₂—O—CH₃, —CH₂—O—CH₂—CH₃, oxo (═O), thioxo (═S), F, Cl, Br, I, —CN, —CF₃, —SF₅, —OH, —O—CH₃, —O—C₂H₅, —O—CH(CH₃)₂, —O—C(CH₃)₃, —NH₂, —NO₂, —O—CF₃, —S—CF₃, —SH, —S—CH₃, —S—C₂H₅, —S—CH(CH₃)₂, —S—C(CH₃)₃, methyl, ethyl, n-propyl, isopropyl, n-butyl, sec-butyl, isobutyl, tert-butyl, n-pentyl, —C(═O)—CH₃, —C(═O)—C₂H₅, —C(═O)—CH(CH₃)₂, —C(═O)—C(CH₃)₃, —C(═O)—OH, —C(═O)—O—CH₃, —C(═O)—O—C₂H₅, —C(═O)—O—CH(CH₃)₂, —C(═O)—O—C(CH₃)₃, —NH—CH₃, —NH—C₂H₅, —NH—C(CH₃)₃, —N(CH₃)₂, —N(C₂H₅)₂, —N(CH₃)(C₂H₅), —NH-phenyl, —N(CH₃)— phenyl, —N(C₂H₅)-phenyl, —N(C₂H₅)-phenyl, —O—CH₂—CH₂—CH₂—CH₃, piperidinyl, pyrrolidinyl, —O—C(═O)—CH₃, —O—C(═O)—C₂Hs, —O—C(═O)—C(CH₃)₃, —(CH₂)-pyridinyl, pyridinyl, —O-phenyl, —O-benzyl, phenyl and benzyl, wherein the cyclic part of the —NH-phenyl, —N(CH₃)-phenyl, —N(C₂H₅)-phenyl, —(CH₂)-pyridinyl, pyridinyl, —O-phenyl, —O-benzyl, phenyl and benzyl residues optionally may be substituted with 1, 2, 3, 4 or 5 substituents independently selected from the group consisting of F, Cl, Br, —OH, —CF₃, —SF₅, —CN, —NO₂, methyl, ethyl, n-propyl, isopropyl, n-butyl, sec-butyl, isobutyl, tert-butyl, —O—CH₃, —O—C₂H₅, —O—CH(CH₃)₂, —O—C(CH₃)₃, —O—CF₃, —S—CF₃, phenyl and —O-benzyl;
   T stands for C—R⁶ and U for C—R⁷ and V for N;
or
T stands for C—R⁶ and U for N and V for C—R⁹;
or
T stands for N and U for C—R⁷ and V for C—R⁹;
or
T stands for N and U for N and V for C—R⁹;
or
T stands for N and U for C—R⁷ and V for N;
or
T stands for C—R⁶ and U for N and V for N;
or
T stands for C—R⁶ and U for C—R⁷ and V for C—R⁹;
R⁶ and R⁷ each independently stand for H; F; Cl; Br; I; —SF₅; —NO₂; —CF₃; —CF₂Cl; —CN; —NH₂; —OH; —SH; —C(═O)—NH₂; —S(═O)₂—NH₂; —C(═O)—OH; —C(═O)—H; —S(═O)₂—OH; —NHR¹¹; —NR¹²R¹³; —OR¹⁴; —SR¹⁵; —C(═O)—NHR¹⁶; —C(═O)—NR¹⁷R¹⁸; —S(═O)₂—NHR¹⁹; —S(═O)₂—NR²⁰R²¹; —C(═O)—OR²²; —C(═O)—R²³, —S(═O)—R²⁴; —S(═O)₂—R²⁴; or
   for an alkyl residue selected from the group consisting of methyl, ethyl, n-propyl, isopropyl, tert-butyl, n-butyl, sec-butyl, isobutyl, n-pentyl, n-hexyl and n-heptyl; or
   for a phenyl residue, which optionally may be bonded via a —(CH═CH)—, —C≡C—, —(CH₂)—, —(CH₂)₂— or —(CH₂)₃ group and/or may be substituted with 1, 2, 3, 4 or 5 substituents independently selected from the group consisting of F, Cl, Br, I, —CN, —CF₃, —SF₅, —OH, —O—CH₃, —O—C₂H₅, —O—CH(CH₃)₂, —O—C(CH₃)₃, —NH₂, —NO₂, —O—CF₃, —S—CF₃, —SH, —S—CH₃, —S—C₂H₅, —S—CH(CH₃)₂, —S—C(CH₃)₃, methyl, ethyl, n-propyl, isopropyl, n-butyl, sec-butyl, isobutyl, tert-butyl and n-pentyl;
R⁹ stands for H; F; Cl; Br; I; —SF₅; —NO₂; —CF₃; —CF₂Cl; —CN; —NH₂; —OH; —SH; —C(═O)—NH₂; —S(═O)₂—NH₂; —C(═O)—NH—OH; —C(═O)—OH; —C(═O)—H; —S(═O)₂—OH; —NHR¹¹; —NR¹²R¹³; —OR¹⁴; —SR¹⁵; —C(═O)—NHR¹⁶; —C(═O)—NR¹⁷R¹⁸; —S(═O)₂—NHR¹⁹; —S(═O)₂—NR²⁰R²¹; —C(═O)—OR²²; —C(═O)—R²³; —S(═O)—R²⁴; —S(═O)₂—R²⁴ or for an alkyl residue selected from the group consisting of methyl, ethyl, n-propyl, isopropyl, tert-butyl, n-butyl, sec-butyl, isobutyl, n-pentyl, n-hexyl and n-heptyl;
R¹¹, R¹², R¹³, R¹⁴, R¹⁵, R¹⁶, R¹⁷, R¹⁸, R¹⁹, R²⁰, R²¹, R²², R²³ and R²⁴ each independently stand for an alkyl residue selected from the group consisting of methyl, ethyl, n-propyl, isopropyl, tert-butyl, n-butyl, sec-butyl, isobutyl, n-pentyl, n-hexyl, n-heptyl, 3-pentyl, 4-heptyl, n-octyl, n-nonyl, 5-nonyl, (2,6)-dimethyl-hept-4-yl, 3-methyl-butyl, n-hexyl, (3,3)-dimethylbutyl, ethenyl, propenyl, 2-butenyl, 3-butenyl, 2-pentenyl and 3-pentenyl;
   for a residue selected from the group consisting of 2,3-dihydro-1H-indenyl, cyclopropyl, oxetanyl, cyclobutyl, cyclopentyl, cyclohexyl, cycloheptyl, cyclopentenyl, cyclohexenyl, cycloheptenyl, imidazolidinyl, tetrahydrofuranyl, tetrahydrothiophenyl, pyrrolidinyl, piperidinyl, morpholinyl, piperazinyl, azepanyl, diazepanyl, azocanyl and thiomorpholinyl, which optionally may be bonded via a —CH₂—O—, —CH₂—CH₂—O—, —CH₂—CH₂—O—CH₂—, —CH₂—CH(CH₃)—O—CH₂, —(CH₂)—, —(CH₂)₂— or —(CH₂)₃ group and/or optionally may be substituted with 1, 2, 3, 4 or 5 substituents independently selected from the group consisting of —CH₂—O—CH₃, —CH₂—O—CH₂—CH₃, oxo (═O), thioxo (═S), F, Cl, Br, I, —CN, —CF₃, —SF₅, —OH, —O—CH₃, —O—C₂H₅, —O—CH(CH₃)₂, —O—C(CH₃)₃, —NH₂, —NO₂, —O—CF₃, —S—CF₃, —SH, —S—CH₃, —S—C₂H₅, —S—CH(CH₃)₂, —S—C(CH₃)₃, methyl, ethyl, n-propyl, isopropyl, n-butyl, sec-butyl, isobutyl, tert-butyl, n-pentyl, —C(═O)—CH₃, —C(═O)—C₂H₅, —C(═O)—CH(CH₃)₂, —C(═O)—C(CH₃)₃, —C(═O)—OH, —C(═O)—O—CH₃, —C(═O)—O—C₂H₅, —C(═O)—O—CH(CH₃)₂, —C(═O)—O—C(CH₃)₃, —NH—CH₃, —NH—C₂H₅, —NH—C(CH₃)₃, —N(CH₃)₂, —N(C₂H₅)₂, —N(CH₃)(C₂H₅), —NH-phenyl, —N(CH₃)-phenyl, —N(C₂H₅)-phenyl, —N(C₂H₅)-phenyl, —O—CH₂—CH₂—CH₂—CH₃, piperidinyl, pyrrolidinyl, —O—C(═O)—CH₃, —O—C(═O)—C₂H₅, —O—C(═O)—C(CH₃)₃, —(CH₂)-pyridinyl, pyridinyl, —O-phenyl, —O-benzyl, phenyl and benzyl, wherein the cyclic part of the —NH-phenyl, —N(CH₃)-phenyl, —N(C₂H₅)-phenyl, —(CH₂)-pyridinyl, pyridinyl, —O-phenyl, —O-benzyl, phenyl and benzyl residues optionally may be substituted with 1, 2, 3, 4 or 5 substituents independently selected from the group consisting of F, Cl, Br, —OH, —CF₃, —SF₅, —CN, —NO₂, methyl, ethyl, n-propyl, isopropyl, n-butyl, sec-butyl, isobutyl, tert-butyl, —O—CH₃, —O—C₂H₅, —O—CH(CH₃)₂, —O—C(CH₃)₃, —O—CF₃, —S—CF₃, phenyl and —O-benzyl; or
   for a residue selected from the group consisting of phenyl, naphthyl, (1,3)-benzodioxolyl, (1,4)-benzodioxanyl, thiophenyl, furanyl, pyrrolyl, pyrazolyl, pyrazinyl, pyranyl, triazolyl, pyridinyl, imidazolyl, indolyl, isoindolyl, benzo[b]furanyl, benzo[b]thiophenyl, benzoxazolyl, benzisoxazolyl, thiazolyl, oxazolyl, isoxazolyl, pyridazinyl, pyrazinyl, pyrimidinyl, indazolyl, quinoxalinyl, quinolinyl and isoquinolinyl, wherein the residue optionally may be bonded via a —(CH₂)—, —(CH₂)₂— or —(CH₂)₃ group and/or optionally may be substituted with 1, 2, 3, 4 or 5 substituents independently selected from the group consisting of F, Cl, Br, I, —CN, —CF₃, —SF₅, —OH, —O—CH₃, —O—C₂H₅, —O—CH(CH₃)₂, —O—C(CH₃)₃, —NH₂, —NO₂, —O—CF₃, —S—CF₃, —SH, —S—CH₃, —S—C₂H₅, —S—CH(CH₃)₂, —S—C(CH₃)₃, methyl, ethyl, n-propyl, isopropyl, n-butyl, sec-butyl, isobutyl, tert-butyl, n-pentyl, —C(═O)—OH, —C(═O)—O—CH₃, —C(═O)—O—C₂H₅, —C(═O)—O—CH(CH₃)₂, —C(═O)—O—C(CH₃)₃, —NH—CH₃, —NH—C₂H₅, —NH—C(CH₃)₃, —N(CH₃)₂, —N(C₂H₅)₂, —N(CH₃)(C₂H₅), —NH—C(═O)—O—CH₃, —NH—C(═O)—O—C₂H₅, —NH—C(═O)—O—C(CH₃)₃, —C(═O)—H, —C(═O)—CH₃, —C(═O)—C₂H₅, —C(═O)—CH(CH₃)₂, —C(═O)—C(CH₃)₃, —C(═O)—NH₂, —C(═O)—NH—CH₃, —C(═O)—NH—C₂H₅, —C(═O)—N(CH₃)₂, —C(═O)—N(C₂H₅)₂, —O-phenyl, —O-benzyl, phenyl and benzyl, wherein the cyclic part of the —O-phenyl, —O-benzyl, phenyl and benzyl residues optionally may be substituted with 1, 2, 3, 4 or 5 substituents independently selected from the group consisting of F, Cl, Br, —OH, —CF₃, —SF₅, —CN, —NO₂, —C₁₋₅-alkyl, —O—C₁₋₅-alkyl, —O—CF₃, —S—CF₃, phenyl and —O-benzyl; or
R¹² and R¹³ together with the nitrogen atom connecting them form a residue selected from the group consisting of 3-aza-bicyclo[3.1.1]heptyl, 6-aza-spiro[2.5]octyl, 3-aza-bicyclo[3.2.1]octyl, 6-aza-bicyclo[3.3.1]heptyl, 8-aza-bicyclo[3.2.1]octyl, 1-oxa-2,8-diaza-spiro[4.5]dec-2-enyl, azocanyl, isoindolyl, indolyl, (1,2,3,6)-tetrahydropyridinyl, (4,5,6,7)-tetrahydroisoxazolo[5,4-c]pyridinyl, pyrrolidinyl, piperidinyl, (1,3,4,5)-tetrahydropyrido[4,3-b]indolyl, (3,4)-dihydro-1H-isoquinolinyl, (1,3,4,9)-tetrahydro-[b]-carbolinyl, imidazolidinyl, (1,3)-thiazolidinyl, piperazinyl, morpholinyl, azepanyl, diazepanyl and thiomorpholinyl, which optionally may be substituted with 1, 2, 3, 4 or 5 substituents independently selected from the group consisting of —CH₂—O—CH₂-oxetanyl, —CH₂—OH, —CH₂—CH₂—OH, ═CH₂, —O—CH₂-oxetanyl, (4,5)-dihydroisoxazolyl, thiazolyl, (1,2,5)-thiadiazolyl, thiophenyl, phenethyl, —CN, —CH₂—N(CH₃)₂, —CH₂—N(C₂H₅)₂, —CH₂—NH—CH₃, —CH₂—NH—C₂H₅, —N—[C(═O)—C₂H₅]-phenyl, —N—[C(═O)—CH₃]-phenyl, —CH₂—O—CH₃, —CH₂—O—CH₂—CH₃, oxo (═O), thioxo (═S), F, Cl, Br, I, —CN, —CF₃, —SF₅, —OH, —O—CH₃, —O—C₂H₅, —O—CH(CH₃)₂, —O—C(CH₃)₃, —NH₂, —NO₂, —O—CF₃, —S—CF₃, —SH, —S—CH₃, —S—C₂H₅, —S—CH(CH₃)₂, —S—C(CH₃)₃, methyl, ethyl, n-propyl, isopropyl, n-butyl, sec-butyl, isobutyl, tert-butyl, n-pentyl, —C(═O)—CH₃, —C(═O)—C₂H₅, —C(═O)—CH(CH₃)₂, —C(═O)—C(CH₃)₃, —C(═O)—OH, —C(═O)—O—CH₃, —C(═O)—O—C₂H₅, —C(═O)—O—CH(CH₃)₂, —C(═O)—O—C(CH₃)₃, —NH—CH₃, —NH—C₂H₅, —NH—C(CH₃)₃, —N(CH₃)₂, —N(C₂H₅)₂, —N(CH₃)(C₂H₅), —NH-phenyl, —N(CH₃)-phenyl, —N(C₂H₅)-phenyl, —N(C₂H₅)-phenyl, —O—CH₂—CH₂—CH₂—CH₃, piperidinyl, pyrrolidinyl, cyclohexyl, cyclopentyl, —O—C(═O)—CH₃, —O—C(═O)—C₂H₅, —O—C(═O)—C(CH₃)₃, —(CH₂)-pyridinyl, pyridinyl, —O-phenyl, —O-benzyl, phenyl and benzyl, wherein the cyclic part of the oxetanyl, (4,5)-dihydroisoxazolyl, thiazolyl, (1,2,5)-thiadiazolyl, thiophenyl, phenethyl, —N—[C(═O)—C₂H₅]-phenyl, —N—[C(═O)—CH₃]-phenyl, —NH-phenyl, —N(CH₃)-phenyl, —N(C₂H₅)-phenyl, —(CH₂)-pyridinyl, pyridinyl, —O-phenyl, —O-benzyl, phenyl and benzyl residues optionally may be substituted with 1, 2, 3, 4 or 5 substituents independently selected from the group consisting of F, Cl, Br, —OH, —CF₃, —SF₅, —CN, —NO₂, methyl, ethyl, n-propyl, isopropyl, n-butyl, sec-butyl, isobutyl, tert-butyl, —O—CH₃, —O—C₂H₅, —O—CH(CH₃)₂, —O—C(CH₃)₃, —O—CF₃, —S—CF₃, phenyl and —O-benzyl;
R²⁵ and R²⁶ each independently stand for hydrogen; or
   for an alkyl residue selected from the group consisting of methyl, ethyl, n-propyl, isopropyl, tert-butyl, n-butyl, sec-butyl, isobutyl, n-pentyl, n-hexyl and n-heptyl; or
   for a residue selected from the group consisting of phenyl, naphthyl, (1,3)-benzodioxolyl, (1,4)-benzodioxanyl, thiophenyl, furanyl, pyrrolyl, pyrazolyl, pyrazinyl, pyranyl, triazolyl, pyridinyl, imidazolyl, indolyl, isoindolyl, benzo[b]furanyl, benzo[b]thiophenyl, benzoxazolyl, benzisoxazolyl, thiazolyl, oxazolyl, isoxazolyl, pyridazinyl, pyrazinyl, pyrimidinyl, indazolyl, quinoxalinyl, quinolinyl and isoquinolinyl, wherein the residue optionally may be bonded via a —(CH₂)—, —(CH₂)₂— or —(CH₂)₃ group and/or optionally may be substituted with 1, 2, 3, 4 or 5 substituents independently selected from the group consisting of F, Cl, Br, I, —CN, —CF₃, —SF₅, —OH, —O—CH₃, —O—C₂H₅, —O—CH(CH₃)₂, —O—C(CH₃)₃, —NH₂, —NO₂, —O—CF₃, —S—CF₃, —SH, —S—CH₃, —S—C₂H₅, —S—CH(CH₃)₂, —S—C(CH₃)₃, methyl, ethyl, n-propyl, isopropyl, n-butyl, sec-butyl, isobutyl, tert-butyl and n-pentyl; or
   for a residue selected from the group consisting of cyclopropyl, cyclobutyl, cyclopentyl, cyclohexyl, cyclopentenyl and cyclohexenyl;
with the proviso that R²⁵ and R²⁶ do not simultaneously stand for hydrogen;
or
R²⁵ and R²⁶ together with the carbon atom connecting them form a residue selected from the group consisting of cyclopropyl, cyclobutyl, cyclopentyl, cyclohexyl, cycloheptyl, cyclopentenyl and cyclohexenyl;
   wherein, unless otherwise specified, the aforementioned alkyl, alkenyl and alkinyl residues each may optionally be substituted with 1, 2, 3, 4, 5, 6, 7, 8 or 9 substituents independently selected from the group consisting of —C(═O)—O—CH₃, —C(═O)—O—C₂H₅, —C(═O)—O—C(CH₃)₃, —O—C(═O)—CH₃, —O—C(═O)—C₂H₅, —O—C(═O)—CH(CH₃)₂, —O—C(═O)—C(CH₃)₃, —O-phenyl, phenyl, F, Cl, Br, I, —CN, —NO₂, —OH, —NH₂, —SH, —O—CH₃, —O—C₂H₅, —O—CH(CH₃)₂, —O—C(CH₃)₃, —S—CH₃, —S—C₂H₅, —S—CH(CH₃)₂, —S—C(CH₃)₃, —NH—CH₃, —NH—C₂Hs, —NH—C(CH₃)₃, —N(CH₃)₂, —N(C₂H₅)₂, —N(CH₃)(C₂H₅), —OCF₃ and —SCF₃; and
R³⁴, R³⁵, R³⁶ and R³⁷ each independently stand for hydrogen or for a linear or branched, saturated or unsaturated aliphatic C₁₋₁₀ residue;
   for an unsaturated or saturated, unsubstituted or mono- or polysubstituted 3-, 4-, 5-, 6-, 7-, 8- or 9-membered cycloaliphatic residue which is respectively bonded to the basic framework via a carbon atom in the ring of the cycloaliphatic residue and which optionally may be condensed with a saturated or unsaturated, unsubstituted or mono- or polysubstituted mono- or polycyclic ring system and/or optionally may be bonded via a linear or branched, unsubstituted or mono- or polysubstituted C₁₋₆-alkylene group or C₂₋₆-alkenylene group or C₂₋₆-alkinylene group; or
   for an unsubstituted or mono- or polysubstituted 5- to 14-membered aryl or heteroaryl residue, which optionally may be condensed with a saturated or unsaturated, unsubstituted or mono- or polysubstituted mono- or polycyclic ring system and/or optionally may be bonded via a linear or branched, unsubstituted or mono- or polysubstituted C₁₋₆-alkylene group or C₂₋₆-alkenylene group or C₂₋₆-alkinylene group;
      in the form of one of their individual stereoisomers, in particular enantiomers or diastereomers, their racemates or in the form of a mixture of stereoisomers, in particular enantiomers and/or diastereomers, in any desired mixture ratio, or respectively in the form of corresponding salts, or respectively in the form of corresponding solvates.

Also preferred are compounds with a dual mode of action corresponding to the formula (V), wherein
X stands for O, S or N—C≡N;
W stands for —CN or —NR³⁴R³⁵;
n stands for 0, 1, 2, 3 or 4;
R¹, R², R³ and R⁴ each independently stand for H; F; Cl; Br; I; —SF₅; —NO₂; —CN; —NH₂; —OH; —SH; —C(═O)—NH₂; —S(═O)₂—NH₂; —C(═O)—NH—OH; —C(═O)—OH; —C(═O)—H; —S(═O)₂—OH; —NHR¹¹; —NR¹²R¹³; —OR¹⁴; —SR¹⁵; —S(═O)—R²⁴; —S(═O)₂—R²⁴ or for a residue selected from the group consisting of methyl, —CF₃, —CCl₃, —CBr₃, —CHF₂, —CH₂F, —CF₂CT, —CCl₂F, ethyl, —CF₂—CH₃, —CH₂—CF₃, —C₂F₅, —CH₂—CCl₃, —CH₂—CBr₃, —CHF—CF₂Cl, —CF₂—CF₂Cl, —CFCl—CF₂Cl, n-propyl, —CF₂—CF₂—CF₃, —CF(CF₃)₂, isopropyl, n-butyl, sec-butyl, isobutyl and tert-butyl;
R⁵ stands for F; Cl; Br; I; —SF₅; —OR¹⁴; —SR¹⁵; —S(═O)—R²⁴; —S(═O)₂—R²⁴; or
   for a residue selected from the group consisting of methyl, —CF₃, —CCl₃, —CBr₃, —CHF₂, —CH₂F, —CF₂Cl, —CCl₂F, —CH₂—CN, —CH₂—O—CH₃, —CH₂—O—CF₃, —CH₂—SF₃, ethyl, —CF₂—CH₃, —CH₂—CF₃, —C₂F₅, —CH₂—CCl₃, —CH₂—CBr₃, —CHF—CF₂Cl, —CF₂—CF₂Cl, —CFCl—CF₂Cl, —CH₂—CH₂—CN, n-propyl, —CF₂—CF₂—CF₃, —CF(CF₃)₂, isopropyl, —CH₂—CH₂—CH₂—CN, —CH₂—O—CH₂—CH₃, —CH₂—CH₂—SF₃, —CH₂—CH₂—OCF₃, —CH(CH₃)(O—CH₃), —CH(CH₃)(S—CH₃), n-butyl, —CF₂—CF₂—CF₂—CF₃, —CH₂—CH₂—CH₂—CH₂—CN, n-butyl, sec-butyl, isobutyl, —C(CH₃)₂(CH₂OH), and tert-butyl; or
   for a residue selected from the group consisting of cyclopropyl, cyclobutyl, cyclopentyl, cyclohexyl, cyclopentenyl and cyclohexenyl, which each optionally may be substituted with 1, 2, 3, 4 or 5 substituents independently selected from the group consisting of oxo (═O), thioxo (═S), —OH, —O—CH₃, —O—C₂H₅, —O—CH(CH₃)₂, —O—C(CH₃)₃, —O—CF₃, —S—CF₃, —SH, —S—CH₃, —S—C₂H₅, —S—CH(CH₃)₂, —S—C(CH₃)₃, methyl, ethyl, n-propyl, isopropyl, n-butyl, sec-butyl, isobutyl, tert-butyl and n-pentyl;
T stands for C—R⁶ and U for C—R⁷ and V for N;
or
T stands for C—R⁶ and U for N and V for C—R⁹;
or
T stands for N and U for C—R⁷ and V for C—R⁹;
or
T stands for N and U for N and V for C—R⁹;
or
T stands for N and U for C—R⁷ and V for N;
or
T stands for C—R⁶ and U for N and V for N;
or
T stands for C—R⁶ and U for C—R⁷ and V for C—R⁹;
R⁶ and R⁷ each independently stand for H; F; Cl; Br; I; —SF₅; —NO₂; —CN; —NH₂; —OH; —SH; —C(═O)—NH₂; —S(═O)₂—NH₂; —C(═O)—NH—OH; —C(═O)—OH; —C(═O)—H; —S(═O)₂—OH; —NHR¹¹; —NR¹²R¹³; —OR¹⁴; —SR¹⁵; —C(═O)—OR²²; —S(═O)—R²⁴; —S(═O)₂—R²⁴; or
   for a residue selected from the group consisting of —CH₂—OH, methyl, —CF₃, —CCl₃, —CBr₃, —CHF₂, —CH₂F, —CF₂Cl, —CCl₂F, ethyl, —CF₂—CH₃, —CH₂—CF₃, —C₂F₅, —CH₂—CCl₃, —CH₂—CBr₃, —CHF—CF₂Cl, —CF₂—CF₂Cl, —CFCl—CF₂Cl, n-propyl, —CF₂—CF₂—CF₃, —CF(CF₃)₂, isopropyl, n-butyl, sec-butyl, isobutyl and tert-butyl; or
   for a phenyl residue, which optionally may be substituted with 1, 2, 3, 4 or 5 substituents independently selected from the group consisting of F, Cl, Br, I, —CN, —CF₃, —SF₅, —OH, —O—CH₃, —O—C₂H₅, —O—CH(CH₃)₂, —O—C(CH₃)₃, —NH₂, —NO₂, —O—CF₃, —S—CF₃, —SH, —S—CH₃, —S—C₂H₅, —S—CH(CH₃)₂, —S—C(CH₃)₃, methyl, ethyl, n-propyl, isopropyl, n-butyl, sec-butyl, isobutyl, tert-butyl and n-pentyl;
R⁹ stands for H; F; Cl; Br; I; —SF₅; —NO₂; —CN; —NH₂; —OH; —SH; —C(═O)—NH₂; —S(═O)₂—NH₂; —C(═O)—NH—OH; —C(═O)—OH; —C(═O)—H; —S(═O)₂—OH; —NHR¹¹; —NR¹²R¹³; —OR¹⁴; —SR¹⁵; —S(═O)—R²⁴; —S(═O)₂—R²⁴ or for a residue selected from the group consisting of methyl, —CF₃, —CCl₃, —CBr₃, —CHF₂, —CH₂F, —CF₂Cl, —CCl₂F, ethyl, —CF₂—CH₃, —CH₂—CF₃, —C₂F₅, —CH₂—CCl₃, —CH₂—CBr₃, —CHF—CF₂Cl, —CF₂—CF₂Cl, —CFCl—CF₂Cl, n-propyl, —CF₂—CF₂—CF₃, —CF(CF₃)₂, isopropyl, n-butyl, sec-butyl, isobutyl and tert-butyl;
R¹¹, R¹², R¹³, R¹⁴, R¹⁵, R²² and R²⁴ each independently stand for a residue selected from the group consisting of methyl, —CF₃, —CCl₃, —CBr₃, —CHF₂, —CH₂F, —CF₂Cl, —CCl₂F, —CH₂—CN, —CH₂—O—CH₃, —CH₂—O—CF₃, —CH₂—SF₃, ethyl, —CF₂—CH₃, —CH₂—CF₃, —C₂F₅, —CH₂—CCl₃, —CH₂—CBr₃, —CHF—CF₂Cl, —CF₂—CF₂Cl, —CFCl—CF₂Cl, —CH₂—CH₂—CN, n-propyl, —CF₂—CF₂—CF₃, —CF(CF₃)₂, isopropyl, —CH₂—CH₂—CH₂—CN, —CH₂—O—CH₂—CH₃, —CH₂—CH₂—SF₃, —CH₂—CH₂—OCF₃, —CH(CH₃)(O—CH₃), —CH(CH₃)(S—CH₃), n-butyl, —CF₂—CF₂—CF₂—CF₃, —CH₂—CH₂—CH₂—CH₂—CN, sec-butyl, isobutyl, tert-butyl, n-pentyl, 3-pentyl, n-heptyl, 4-heptyl, n-octyl, n-nonyl, 5-nonyl, (2,6)-dimethyl-hept-4-yl, 3-methyl-butyl, n-hexyl, (3,3)-dimethylbutyl, —CH₂—CH₂—O—CH₃, —CH₂—CH₂—O—C₂H₅, —CH₂—CH₂—O-phenyl, —CH₂—CH₂—CH₂—O—CH₃, ethenyl, propenyl, 2-butenyl, 3-butenyl, 2-pentenyl and 3-pentenyl; or
   for a residue selected from the group consisting of 2,3-dihydro-1H-indenyl, cyclopropyl, oxetanyl, cyclobutyl, cyclopentyl, cyclohexyl, imidazolidinyl, tetrahydrofuranyl, tetrahydrothiophenyl, pyrrolidinyl, piperidinyl, morpholinyl, piperazinyl, azepanyl, diazepanyl, azocanyl and thiomorpholinyl, which each optionally may be bonded via a —CH₂—O—, —CH₂—CH₂—O—, —CH₂—CH₂—O—CH₂—, —CH₂—CH(CH₃)—O—CH₂—, —(CH₂)—, —(CH₂)₂— or —(CH₂)₃ group and/or optionally may be substituted with 1, 2, 3, 4 or 5 substituents independently selected from the group consisting of oxo (═O), thioxo (═S), —OH, —O—CH₃, —O—C₂H₅, —O—CH(CH₃)₂, —O—C(CH₃)₃, —O—CF₃, —S—CF₃, —SH, —S—CH₃, —S—C₂H₅, —S—CH(CH₃)₂, —S—C(CH₃)₃, methyl, ethyl, n-propyl, isopropyl, n-butyl, sec-butyl, isobutyl, tert-butyl, n-pentyl, —C(═O)—CH₃, —C(═O)—C₂H₅, —C(═O)—CH(CH₃)₂, —C(═O)—C(CH₃)₃, —C(═O)—OH, —C(═O)—O—CH₃, —C(═O)—O—C₂H₅, —C(═O)—O—CH(CH₃)₂ and —C(═O)—O—C(CH₃)₃; or
   for a residue selected from the group consisting of phenyl, naphthyl, thiophenyl, furanyl, pyrrolyl, pyrazolyl, pyrazinyl, pyranyl, triazolyl, pyridinyl, imidazolyl, thiazolyl, oxazolyl and isoxazolyl, wherein the residue optionally may be bonded via a —(CH₂)—, —(CH₂)₂— or —(CH₂)₃ group and/or optionally may be substituted with 1, 2, 3, 4 or 5 substituents independently selected from the group consisting of F, Cl, Br, I, —CN, —CF₃, —SF₅, —OH, —O—CH₃, —O—C₂H₅, —O—CH(CH₃)₂, —O—C(CH₃)₃, —N112, —NO₂, —O—CF₃, —S—CF₃, —SH, —S—CH₃, —S—C₂H₅, —S—CH(CH₃)₂, —S—C(CH₃)₃, methyl, ethyl, n-propyl, isopropyl, n-butyl, sec-butyl, isobutyl, tert-butyl, n-pentyl, —C(═O)—OH, —C(═O)—O—CH₃, —C(═O)—O—C₂H₅, —C(═O)—O—CH(CH₃)₂, —C(═O)—O—C(CH₃)₃, —NH—CH₃, —NH—C₂H₅, —NH—C(CH₃)₃, —N(CH₃)₂, —N(C₂H₅)₂, —N(CH₃)(C₂H₅), —NH—C(═O)—O—CH₃, —NH—C(═O)—O—C₂H₅, —NH—C(═O)—O—C(CH₃)₃, —C(═O)—H, —C(═O)—CH₃, —C(═O)—C₂H₅, —C(═O)—CH(CH₃)₂, —C(═O)—C(CH₃)₃, —C(═O)—NH₂, —C(═O)—NH—CH₃, —C(═O)—NH—C₂Hs, —C(═O)—N(CH₃)₂, —C(═O)—N(C₂H₅)₂, —O-phenyl, —O-benzyl, phenyl and benzyl; or
R¹² and R¹³ together with the carbon atom connecting them form a residue selected from the group consisting of 3-aza-bicyclo[3.1.1]heptyl 3-aza-bicyclo[3.1.1]heptyl, 6-aza-spiro[2.5]octyl, 3-aza-bicyclo[3.2.1]octyl, 6-aza-bicyclo[3.3.1]heptyl, 8-aza-bicyclo[3.2.1]octyl, 1-oxa-2,8-diaza-spiro[4.5]dec-2-enyl, azocanyl, isoindolyl, indolyl, (1,2,3,6)-tetrahydropyridinyl, (4,5,6,7)-tetrahydroisoxazolo-[5,4-c]pyridinyl, pyrrolidinyl, piperidinyl, piperazinyl, morpholinyl, azepanyl, diazepanyl and thiomorpholinyl, which each optionally may be substituted with 1, 2, 3, 4 or 5 substituents independently selected from the group consisting of —CH₂—O—CH₂-oxetanyl, —CH₂—OH, —CH₂—CH₂—OH, ═CH₂, —O—CH₂-oxetanyl, (4,5)-dihydroisoxazolyl, thiazolyl, (1,2,5)-thiadiazolyl, thiophenyl, phenethyl, —CN, —CH₂—N(CH₃)₂, —CH₂—N(C₂H₅)₂, —CH₂—NH—CH₃, —CH₂—NH—C₂H₅, —N—[C(═O)—C₂H₅]-phenyl, —N—[C(═O)—CH₃]-phenyl, —CH₂—O—CH₃, —CH₂—O—CH₂—CH₃, oxo (═O), thioxo (═S), F, Cl, Br, I, —CN, —CF₃, —SF₅, —OH, —O—CH₃, —O—C₂H₅, —O—CH(CH₃)₂, —O—C(CH₃)₃, —NH₂, —NO₂, —O—CF₃, —S—CF₃, —SH, —S—CH₃, —S—C₂HS, —S—CH(CH₃)₂, —S—C(CH₃)₃, methyl, ethyl, n-propyl, isopropyl, n-butyl, sec-butyl, isobutyl, tert-butyl, n-pentyl, —C(═O)—CH₃, —C(═O)—C₂Hs, —C(═O)—CH(CH₃)₂, —C(═O)—C(CH₃)₃, —C(═O)—OH, —C(═O)—O—CH₃, —C(═O)—O—C₂H₅, —C(═O)—O—CH(CH₃)₂, —C(═O)—O—C(CH₃)₃, —NH—CH₃, —NH—C₂H₅, —NH—C(CH₃)₃, —N(CH₃)₂, —N(C₂H₅)₂, —N(CH₃)(C₂H₅), —NH-phenyl, —N(CH₃)-phenyl, —N(C₂H₅)-phenyl, —N(C₂H₅)-phenyl, —O—CH₂—CH₂—CH₂—CH₃, cyclohexyl, cyclopentyl, piperidinyl, pyrrolidinyl, —O—C(═O)—CH₃, —O—C(═O)—C₂H₅, —O—C(═O)—C(CH₃)₃, —(CH₂)-pyridinyl, pyridinyl, —O-phenyl, —O-benzyl, phenyl and benzyl, wherein the cyclic part of the oxetanyl, (4,5)-dihydroisoxazolyl, thiazolyl, (1,2,5)-thiadiazolyl, thiophenyl, phenethyl, —N—[C(═O)—C₂H₅]-phenyl, —N—[C(═O)—CH₃]-phenyl, —NH-phenyl, —N(CH₃)-phenyl, —N(C₂H₅)-phenyl, —(CH₂)-pyridinyl, pyridinyl, —O-phenyl, —O-benzyl, phenyl and benzyl residues optionally may be substituted with 1, 2, 3, 4 or 5 substituents independently selected from the group consisting of —CF₃, F, Cl, Br, —OH, —CF₃, —SF₅, —CN, —NO₂, methyl, ethyl, n-propyl, isopropyl, n-butyl, sec-butyl, isobutyl, tert-butyl, —O—CH₃, —O—C₂H₅, —O—CH(CH₃)₂, —O—C(CH₃)₃, —O—CF₃, —S—CF₃, phenyl and —O-benzyl;
R²⁵ and R²⁶ each independently stand for hydrogen; or
   for an alkyl residue selected from the group consisting of —CH₂—OH, —CH₂—CH₂—OH, —CH₂—CH₂—CH₂—OH, —CH₂—CH₂—CH₂—CH₂—OH, isopropyl, n-butyl, sec-butyl, isobutyl, methyl, ethyl and n-propyl; or
   for a residue selected from the group consisting of phenyl, naphthyl, thiophenyl, furanyl, pyrrolyl, pyrazolyl, pyrazinyl, pyranyl, triazolyl, pyridinyl, imidazolyl, indolyl, isoindolyl, thiazolyl, oxazolyl, isoxazolyl, pyridazinyl, pyrazinyl, pyrimidinyl, indazolyl, quinoxalinyl, quinolinyl and isoquinolinyl, which each optionally may be bonded via a —(CH₂)—, —(CH₂)₂— or —(CH₂)₃ group and/or optionally may be substituted with 1, 2, 3, 4 or 5 substituents independently selected from the group consisting of F, Cl, Br, I, —CN, —CF₃, —SF₅, —OH, —O—CH₃, —O—C₂H₅, —O—CH(CH₃)₂, —O—C(CH₃)₃, —NH₂, —NO₂, —O—CF₃, —S—CF₃, —SH, —S—CH₃, —S—C₂H₅, —S—CH(CH₃)₂, —S—C(CH₃)₃, methyl, ethyl, n-propyl, isopropyl, n-butyl, sec-butyl, isobutyl, tert-butyl and n-pentyl; or
   for a residue selected from the group consisting of cyclopropyl, cyclobutyl, cyclopentyl and cyclohexyl;
with the proviso that R²⁵ and R²⁶ do not simultaneously stand for hydrogen;
or
R²⁵ and R²⁶ together with the carbon atom connecting them form a residue selected from the group consisting of cyclopropyl, cyclobutyl, cyclopentyl, cyclohexyl and cycloheptyl; and
R³⁴ and R³⁵ each independently stand for hydrogen, or for a linear or branched, saturated or unsaturated aliphatic C₁₋₁₀ residue; or
   for an unsaturated or saturated, unsubstituted or mono- or polysubstituted 3-, 4-, 5-, 6-, 7-, 8- or 9-membered cycloaliphatic residue, which is respectively bonded to the basic framework via a carbon atom in the ring of the cycloaliphatic residue and which optionally may be condensed with a saturated or unsaturated, unsubstituted or mono- or polysubstituted mono- or polycyclic ring system and/or optionally may be bonded via a linear or branched, unsubstituted or mono- or polysubstituted C₁₋₆-alkylene group or C₂₋₆-alkenylene group or C₂₋₆-alkinylene group; or
   for an unsubstituted or mono- or polysubstituted 5- to 14-membered aryl or heteroaryl residue, which optionally may be condensed with a saturated or unsaturated, unsubstituted or mono- or polysubstituted mono- or polycyclic ring system and/or optionally may be bonded via a linear or branched, unsubstituted or mono- or polysubstituted C₁₋₆-alkylene group or C₂₋₆-alkenylene group or C₂₋₆-alkinylene group;
      in the form of one of their individual stereoisomers, in particular enantiomers or diasteromers, their racemates or in the form of a mixture of stereoisomers, in particular enantiomers and/or diastereomers, in any desired mixture ratio, or respectively in the form of corresponding salts, or respectively in the form of corresponding solvates.

Additionally preferred are compounds with a dual mode of action corresponding to the formula (V), wherein
X stands for O, S or N—C≡N;
W stands for —NR³⁴R³⁵;
n stands for 0, 1 or 2;
R¹, R³ and R⁴ each independently stand for H; F; Cl; Br; or for a residue selected from the group consisting of methyl, —CF₃, —CCl₃, —CBr₃, —CHF₂, —CH₂F, —CF₂Cl, —CCl₂F, ethyl, —CF₂—CH₃, —CH₂—CF₃, —C₂F₅, —CH₂—CCl₃, —CH₂—CBr₃, —CHF—CF₂Cl, —CF₂—CF₂Cl and —CFCl—CF₂Cl;
R² stands for F; Cl; Br; I or for a residue selected from the group consisting of methyl, —CF₃, —CCl₃, —CBr₃, —CHF₂, —CH₂F, —CF₂Cl, —CCl₂F, ethyl, —CF₂—CH₃, —CH₂—CF₃, —C₂F₅, —CH₂—CCl₃, —CH₂—CBr₃, —CHF—CF₂Cl, —CF₂—CF₂Cl, —CFCl—CF₂Cl, n-propyl, —CF₂—CF₂—CF₃, —CF(CF₃)₂, isopropyl, sec-butyl, isobutyl, tert-butyl, —O—CH₃, —O—CF₃, —O—CCl₃, —O—CBr₃, —O—CHF₂, —O—CH₂F, —O—CF₂Cl, —O—CCl₂F, —O—C₂H₅, —O—CF₂—CH₃, —O—CH₂—CF₃, —O—C₂F₅, —O—CH₂—CCl₃, —O—CH₂—CBr₃, —O—CHF—CF₂Cl, —O—CF₂—CF₂Cl, —O—CFCl—CF₂Cl, —O—CH₂—CH₂—CH₃, —O—CF₂—CF₂—CF₃, —O—CF(CF₃)₂, —O—CH(CH₃)₂, —O—C(CH₃)₃, —S—CH₃, —S—CF₃, —S—CCl₃, —S—CBr₃, —S—CHF₂, —S—CH₂F, —S—CF₂Cl, —S—CCl₂F, —S—C₂H₅, —S—CF₂—CH₃, —S—CH₂—CF₃, —S—C₂F₅, —S—CH₂—CCl₃, —S—CH₂—CBr₃, —S—CHF—CF₂Cl, —S—CF₂—CF₂Cl, —S—CFCl—CF₂Cl, —S—CH₂—CH₂—CH₃, —S—CF₂—CF₂—CF₃, —S—CF(CF₃)₂, —S—CH(CH₃)₂ and —S—C(CH₃)₃;
R⁵ stands for F; Cl; Br; I; —SF₅; or
   for a residue selected from the group consisting of methyl, ethyl, —CF₃, —CCl₃, —CBr₃, —CHF₂, —CH₂F, —CF₂Cl, —CCl₂F, —CF₂—CH₃, —CH₂—CF₃, —C₂F₅, —CH₂—CCl₃, —CH₂—CBr₃, —CHF—CF₂Cl, —CF₂—CF₂Cl, —CFCl—CF₂Cl, —CF₂—CF₂—CF₃, —CF(CF₃)₂, sec-butyl, isobutyl, —C(CH₃)₂(CH₂OH), tert-butyl, —O—CF₃, —O—CCl₃, —O—CBr₃, —O—CHF₂, —O—CH₂F, —O—CF₂Cl, —O—CCl₂F, —O—CF₂—CH₃, —O—CH₂—CF₃, —O—C₂F₅, —O—CH₂—CCl₃, —O—CH₂—CBr₃, —O—CHF—CF₂Cl, —O—CF₂—CF₂Cl, —O—CFCl—CF₂Cl, —O—CF₂—CF₂—CF₃, —O—CF(CF₃)₂, —O—CH(CH₃)₂, —O—C(CH₃)₃, —S—CF₃, —S—CCl₃, —S—CBr₃, —S—CHF₂, —S—CH₂F, —S—CF₂Cl, —S—CCl₂F, —S—CF₂—CH₃, —S—CH₂—CF₃, —S—C₂F₅, —S—CH₂—CCl₃, —S—CH₂—CBr₃, —S—CHF—CF₂Cl, —S—CF₂—CF₂Cl, —S—CFCl—CF₂Cl, —S—CF₂—CF₂—CF₃, —S—CF(CF₃)₂, —S—CH(CH₃)₂, —S—C(CH₃)₃, —S(═O)₂—CF₃, —S(═O)₂—CCl₃, —S(═O)₂—CBr₃, —S(═O)₂—CHF₂, —S(═O)₂—CH₂F, —S(═O)₂—CF₂Cl, —S(═O)₂—CCl₂F, —S(═O)₂—CF₂—CH₃, —S(═O)₂—CH₂—CF₃, —S(═O)₂—C₂F₅, —S(═O)₂—CH₂—CCl₃, —S(═O)₂—CH₂—CBr₃, —S(═O)₂—CHF—CF₂Cl, —S(═O)₂—CF₂—CF₂Cl, —S(═O)₂—CFCl—CF₂Cl, —S(═O)₂—CF₂—CF₂—CF₃, —S(═O)₂—CF(CF₃)₂, —S(═O)₂—CH(CH₃)₂ and —S(═O)₂—C(CH₃)₃; or
   for a residue selected from the group consisting of cyclopropyl, cyclobutyl, cyclopentyl, cyclohexyl, cyclopentenyl and cyclohexenyl;
T stands for C—R⁶ and U for C—R⁷ and V for N;
or
T stands for C—R⁶ and U for N and V for C—R⁹;
or
T stands for N and U for C—R⁷ and V for C—R⁹;
or
T stands for N and U for N and V for C—R⁹;
or
T stands for N and U for C—R⁷ and V for N;
or
T stands for C—R⁶ and U for N and V for N;
or
T stands for C—R⁶ and U for C—R⁷ and V for C—R⁹;
R⁶ and R⁷ each independently stand for H; F; Cl; Br; I; —NO₂; —CN; —C(═O)—OCH₃; —C(═O)—OC₂H₅; or
   for a residue selected from the group consisting of —CH₂—OH, methyl, —CF₃, —CCl₃, —CBr₃, —CHF₂, —CH₂F, —CF₂Cl, —CCl₂F, ethyl, n-propyl, isopropyl, sec-butyl, isobutyl and tert-butyl or for a phenyl residue, which optionally may be substituted with 1, 2, 3, 4 or 5 substituents independently selected from the group consisting of F, Cl, Br, I, —CN, —CF₃, —SF₅, —OH, —O—CH₃, —O—C₂H₅, —O—CH(CH₃)₂, —O—C(CH₃)₃, —NH₂, —NO₂, —O—CF₃, —S—CF₃, —SH, —S—CH₃, —S—C₂H₅, —S—CH(CH₃)₂, —S—C(CH₃)₃, methyl, ethyl, n-propyl, isopropyl, n-butyl, sec-butyl, isobutyl, tert-butyl and n-pentyl;
R⁹ stands for H; F; Cl; Br; I; —NO₂; —CN; or for a residue selected from the group consisting of methyl, —CF₃, —CCl₃, —CBr₃, —CHF₂, —CH₂F, —CF₂Cl, —CCl₂F, ethyl n-propyl, —CF₂—CF₂—CF₃, —CF(CF₃)₂, isopropyl, sec-butyl, isobutyl and tert-butyl;
R²⁵ and R²⁶ each independently stand for hydrogen; or
   for an alkyl residue selected from the group consisting of —CH₂—OH, —CH₂—CH₂—OH, —CH₂—CH₂—CH₂—OH, —CH₂—CH₂—CH₂—CH₂—OH, isopropyl, n-butyl, sec-butyl, isobutyl, methyl, ethyl and n-propyl or for a residue selected from the group consisting of phenyl, benzyl, phenethyl, cyclopropyl, cyclobutyl, cyclopentyl and cyclohexyl;
with the proviso that R²⁵ and R²⁶ do not simultaneously stand for hydrogen;
or
R²⁵ and R²⁶ together with the carbon atom connecting them form a residue selected from the group consisting of cyclopropyl, cyclobutyl, cyclopentyl, cyclohexyl and cycloheptyl; and
R³⁴ and R³⁵ each independently stand for hydrogen, or for a linear or branched, saturated or unsaturated aliphatic C₁₋₁₀ residue; or
   for an unsaturated or saturated, unsubstituted or mono- or polysubstituted 3-, 4-, 5-, 6-, 7-, 8- or 9-membered cycloaliphatic residue, which is respectively bonded to the basic framework via a carbon atom in the ring of the cycloaliphatic residue and which optionally may be condensed with a saturated or unsaturated, unsubstituted or mono- or polysubstituted mono- or polycyclic ring system and/or optionally may be bonded via a linear or branched, unsubstituted or mono- or polysubstituted C₁₋₆-alkylene group or C₂₋₆-alkenylene group or C₂₋₆-alkinylene group;
      in the form of one of their individual stereoisomers, in particular enantiomers or diastereomers, their racemates or in the form of a mixture of stereoisomers, in particular enantiomers and/or diastereomers, in any desired mixture ratio, or respectively in the form of corresponding salts, or respectively in the form of corresponding solvates.

Also preferred are compounds with a dual mode of action corresponding to the formula (V), wherein
X stands for O or S;
W stands for —NR³⁴R³⁵;
n stands for 0, 1 or 2;
R¹, R³ and R⁴ each stand for H;
R² stands for F; Cl; Br; I or for a residue selected from the group consisting of methyl, —CF₃, —CCl₃, —CBr₃, —CHF₂, —CH₂F, —O—CH₃, —O—CF₃, —O—CCl₃, —O—CBr₃, —O—CHF₂, —O—CH₂F, —S—CH₃, —S—CF₃, —S—CCl₃, —S—CBr₃, —S—CHF₂, —S—CH₂F, —S—CF₂Cl and —S—CCl₂F;
R⁵ stands for F; Cl; Br; I; —SF₅; or
   for a residue selected from the group consisting of methyl, ethyl, —CF₃, —CCl₃, —CBr₃, —CHF₂, —CH₂F, —CF₂Cl, —CCl₂F, —C(CH₃)₂(CH₂OH), tert-butyl, —O—CF₃, —O—CCl₃, —O—CBr₃, —O—CHF₂, —O—CH₂F, —O—CF₂Cl, —O—CCl₂F, —O—CF₂—CH₃, —S—CF₃, —S—CCl₃, —S—CBr₃, —S—CHF₂, —S—CH₂F, —S—CF₂Cl, —S—CCl₂F, —S—CF₂—CH₃, —S(═O)₂—CF₃, —S(═O)₂—CCl₃, —S(═O)₂—CBr₃, —S(═O)₂—CHF₂, —S(═O)₂—CH₂F and —S(═O)₂—CF₂Cl; or
   for a residue selected from the group consisting of cyclopropyl, cyclobutyl, cyclopentyl, cyclohexyl, cyclopentenyl and cyclohexenyl;
T stands for C—R⁶ and U for C—R⁷ and V for N;
or
T stands for C—R⁶ and U for N and V for C—R⁹;
or
T stands for N and U for C—R⁷ and V for C—R⁹;
or
T stands for N and U for N and V for C—R⁹;
or
T stands for N and U for C—R⁷ and V for N;
or
T stands for C—R⁶ and U for N and V for N;
or
T stands for C—R⁶ and U for C—R⁷ and V for C—R⁹;
R⁶ and R⁷ each independently stand for —CF₃; phenyl; —C(═O)—OCH₃; —C(═O)—OC₂H₅; methyl; —CH₂—OH; H; F; Cl; Br and I;
R⁹ stands for —CF₃; H; F; Cl; Br or I; and
R²⁵ and R²⁶ each independently stand for hydrogen;
   for an alkyl residue selected from the group consisting of —CH₂—OH, —CH₂—CH₂—OH, —CH₂—CH₂—CH₂—OH, —CH₂—CH₂—CH₂—CH₂—OH, isopropyl, n-butyl, sec-butyl, isobutyl, methyl, ethyl and n-propyl; or
   for a residue selected from the group consisting of phenyl, benzyl, phenethyl, cyclopropyl, cyclobutyl, cyclopentyl and cyclohexyl;
with the proviso that R²⁵ and R²⁶ do not simultaneously stand for hydrogen; or
R²⁵ and R²⁶ together with the carbon atom connecting them form a residue selected from the group consisting of cyclopropyl, cyclobutyl, cyclopentyl, cyclohexyl and cycloheptyl; and
R³⁴ and R³⁵ each independently stand for hydrogen, or for a linear or branched, saturated or unsaturated aliphatic C₁₋₁₀ residue; or
   for an unsaturated or saturated, unsubstituted or mono- or polysubstituted 3-, 4-, 5-, 6-, 7-, 8- or 9-membered cycloaliphatic residue, which is respectively bonded to the basic framework via a carbon atom in the ring of the cycloaliphatic residue and which optionally may be condensed with a saturated or unsaturated, unsubstituted or mono- or polysubstituted mono- or polycyclic ring system and/or optionally may be bonded via a linear or branched, unsubstituted or mono- or polysubstituted C₁₋₆-alkylene group or C₂₋₆-alkenylene group or C₂₋₆-alkinylene group;
      in the form of one of their individual stereoisomers, in particular enantiomers or diastereomers, their racemates or in the form of a mixture of stereoisomers, in particular enantiomers and/or diastereomers, in any desired mixture ratio, or respectively in the form of corresponding salts, or respectively in the form of corresponding solvates.

Also preferred are compounds with a dual mode of action corresponding to the foregoing formula (V), wherein
X stands for O;
W stands for —NR³⁴R³⁵;
n stands for 1;
R¹, R³ and R⁴ respectively stand for H;
R² stands for methyl; —O—CH₃; F; Cl; Br or I;
R⁵ stands for a residue selected from the group consisting of methyl, ethyl, —CF₃, —CCl₃, —CBr₃, —CHF₂, —CH₂F, —CF₂Cl, —CCl₂F, —C(CH₃)₂(CH₂OH), tert-butyl, —O—CF₃, —O—CCl₃, —O—CBr₃, —O—CHF₂, —O—CH₂F, —S—CF₃, —S—CCl₃, —S—CBr₃, —S—CHF₂ and —S—CH₂F; or
   for a residue selected from the group consisting of cyclopropyl, cyclobutyl, cyclopentyl, cyclohexyl, cyclopentenyl and cyclohexenyl;
T stands for CH and U for CH and V for N;
or
T stands for CH and U for N and V for CH;
or
T stands for N and U for CH and V for CH;
or
T stands for N and U for N and V for CH;
or
T stands for N and U for CH and V for N;
or
T stands for CH and U for N and V for N;
or
T stands for CH and U for CH and V for CH;
R²⁵ stands for an alkyl residue selected from the group consisting of —CH₂—OH, —CH₂—CH₂—OH, methyl, ethyl and n-propyl or for a residue selected from the group consisting of benzyl, phenyl, phenethyl, cyclopropyl, cyclobutyl, cyclopentyl and cyclohexyl;
R²⁶ stands for hydrogen or for a residue selected from the group consisting of methyl, ethyl and n-propyl; or
R²⁵ and R²⁶ together with the carbon atom connecting them form a residue selected from the group consisting of cyclopropyl, cyclobutyl, cyclopentyl and cyclohexyl; and
R³⁴ and R³⁵ each independently stand for hydrogen, or for a linear or branched, saturated or unsaturated aliphatic C₁₋₁₀ residue;
   in the form of one of their individual stereoisomers, in particular enantiomers or diastereomers, their racemates or in the form of a mixture of stereoisomers, in particular enantiomers and/or diastereomers, in any desired mixture ratio, or respectively in the form of corresponding salts, or respectively in the form of corresponding solvates.

In a further preferred embodiment, at least one compound can be used for the production of a drug for the treatment of pain that is selected from the group consisting of:
N-((2-(4-(dimethylamino)-4-phenylpiperidin-1-yl)-6-(trifluoromethyl)pyridin-3-yl)methyl)-2-(3-fluoro-4-(methylsulfonamido)phenyl)propanamide;
N-((6-tert-butyl-2-(4-(dimethylamino)-4-phenylpiperidin-1-yl)pyridin-3-yl)methyl)-2-(3-fluoro-4-(methylsulfonamido)phenyl)propanamide;
N-((2-(4-benzyl-4-(dimethylamino)piperidin-1-yl)-6-(trifluoromethyl)pyridin-3-yl)methyl)-2-(3-fluoro-4-(methylsulfonamido)phenyl)propanamide;
N-((2-(4-benzyl-4-(dimethylamino)piperidin-1-yl)-6-tert-butylpyridin-3-yl)methyl)-2-(3-fluoro-4-(methylsulfonamido)phenyl)propanamide;
N-(2-(4-benzyl-4-(dimethylamino)piperidin-1-yl)-4-(trifluoromethyl)benzyl)-2-(3-fluoro-4-(methylsulfonamido)phenyl)propanamide;
N-(2-(4-benzyl-4-(dimethylamino)piperidin-1-yl)-4-(trifluoromethyl)benzyl)-2-(3-methyl-4-(methylsulfonamido)phenyl)propanamide;
N-((2-(4-(dimethylamino)-4-(3-fluorobenzyl)piperidin-1-yl)-6-(trifluoromethyl)pyridin-3-yl)methyl)-2-(3-fluoro-4-(methylsulfonamido)-phenyl)propanamide;
N-(2-(4-(dimethylamino)-4-(3-fluorobenzyl)piperidin-1-yl)-4-(trifluoromethyl)-benzyl)-2-(3-fluoro-4-(methylsulfonamido)phenyl)propanamide, and
N-(2-(4-(dimethylamino)-4-(3-fluorobenzyl)piperidin-1-yl)-4-(trifluoromethyl)-benzyl)-2-(3-methyl-4-(methylsulfonamido)phenyl)propanamide;
   in the form of one of its individual stereoisomers, in particular enantiomers or diastereomers, its racemates or in the form of a mixture of stereoisomers, in particular enantiomers and/or diastereomers, in any desired mixture ratio, or respectively in the form of corresponding salts, or respectively in the form of corresponding solvates.

In a further preferred embodiment, at least one compound can be used for the production of a drug for the treatment of pain that is selected from the group consisting of
N-((2-(4-(dimethylamino)-4-(thiophen-2-yl)piperidin-1-yl)-6-(trifluoromethyl)pyridin-3-yl)methyl)-2-(3-fluoro-4-(methylsulfonamido)phenyl)propanamide, and
N-(2-(4-(dimethylamino)-4-(thiophen-2-yl)piperidin-1-yl)-4-(trifluoromethyl)benzyl)-2-(3-fluoro-4-(methylsulfonamido)phenyl)propanamide;
   in the form of one of its individual stereoisomers, in particular enantiomers or diastereomers, its racemates or in the form of a mixture of stereoisomers, in particular enantiomers and/or diastereomers, in any desired mixture ratio, or respectively in the form of corresponding salts, or respectively in the form of corresponding solvates.

In a likewise preferred embodiment
i) a combination comprising at least one compound that has affinity to the μ-opioid receptor and at least one compound that has affinity to the VR1 receptor, or
ii) at least one compound that has affinity to the μ-opioid receptor and VR1 receptor,
   wherein the μ-opioid receptor affinity amounts to ≦5.0 μM (Kᵢ value, human) and the VR1 receptor affinity amounts to ≦5.0 μM (Kᵢ value, human), can be used for the production of a drug for the treatment of overactive bladder syndrome, coughing, asthma, COPD and/or diabetes. In this case, at least one compound corresponding to formula (I), (II), (III) and/or (IV) can preferably be used. It is particularly preferred if the at least one compound that can be used is selected from the group consisting of:
N-((2-(4-(dimethylamino)-4-phenylpiperidin-1-yl)-6-(trifluoromethyl)pyridin-3-yl)methyl)-2-(3-fluoro-4-(methylsulfonamido)phenyl)propanamide;
N-((6-tert-butyl-2-(4-(dimethylamino)-4-phenylpiperidin-1-yl)pyridin-3-yl)methyl)-2-(3-fluoro-4-(methylsulfonamido)phenyl)propanamide;
N-((2-(4-benzyl-4-(dimethylamino)piperidin-1-yl)-6-(trifluoromethyl)pyridin-3-yl)methyl)-2-(3-fluoro-4-(methylsulfonamido)phenyl)propanamide;
N-((2-(4-benzyl-4-(dimethylamino)piperidin-1-yl)-6-tert-butylpyridin-3-yl)methyl)-2-(3-fluoro-4-(methylsulfonamido)phenyl)propanamide;
N-(2-(4-benzyl-4-(dimethylamino)piperidin-1-yl)-4-(trifluoromethyl)benzyl)-2-(3-fluoro-4-(methylsulfonamido)phenyl)propanamide;
N-(2-(4-benzyl-4-(dimethylamino)piperidin-1-yl)-4-(trifluoromethyl)benzyl)-2-(3-methyl-4-(methylsulfonamido)phenyl)propanamide;
N-((2-(4-(dimethylamino)-4-(3-fluorobenzyl)piperidin-1-yl)-6-(trifluoromethyl)pyridin-3-yl)methyl)-2-(3-fluoro-4-(methylsulfonamido)phenyl)propanamide;
N-(2-(4-(dimethylamino)-4-(3-fluorobenzyl)piperidin-1-yl)-4-(trifluoromethyl)benzyl)-2-(3-fluoro-4-(methylsulfonamido)phenyl)propanamide; and
N-(2-(4-(dimethylamino)-4-(3-fluorobenzyl)piperidin-1-yl)-4-(trifluoromethyl)benzyl)-2-(3-methyl-4-(methylsulfonamido)phenyl)propanamide;
   in the form of one of its individual stereoisomers, in particular enantiomers or diastereomers, its racemates or in the form of a mixture of stereoisomers, in particular enantiomers and/or diastereomers, in any desired mixture ratio, or respectively in the form of corresponding salts, or respectively in the form of corresponding solvates.

A compound corresponding to formula (V) can also preferably be used to produce a drug for the treatment of overactive bladder syndrome, coughing, asthma, COPD and/or diabetes. In this case it is particularly preferred to use a compound selected from the group consisting of:
N-((2-(4-(dimethylamino)-4-(thiophen-2-yl)piperidin-1-yl)-6-(trifluoromethyl)pyridin-3-yl)methyl)-2-(3-fluoro-4-(methylsulfonamido)phenyl)propanamide; and
N-(2-(4-(dimethylamino)-4-(thiophen-2-yl)piperidin-1-yl)-4-(trifluoromethyl)benzyl)-2-(3-fluoro-4-(methylsulfonamido)phenyl)propanamide;
   in the form of one of its individual stereoisomers, in particular enantiomers or diastereomers, its racemates or in the form of a mixture of stereoisomers, in particular enantiomers and/or diastereomers, in any desired mixture ratio, or respectively in the form of corresponding salts, or respectively in the form of corresponding solvates.

The invention additionally relates to a pharmaceutical composition containing at least one compound corresponding to formula (I), (II), (III) or (IV) respectively defined as above. The pharmaceutical composition can also preferably comprise at least one compound corresponding to formula (V) respectively defined as above. The foregoing definitions corresponding to formulas (I), (II), (III), (IV) and (V) including their preferred embodiments may also be applied accordingly to the compounds contained in the pharmaceutical composition of the present invention.

Particularly preferred are pharmaceutical compositions that contain at least one compound selected from the group consisting of:
N-((2-(4-(dimethylamino)-4-phenylpiperidin-1-yl)-6-(trifluoromethyl)pyridin-3-yl)methyl)-2-(3-fluoro-4-(methylsulfonamido)phenyl)propanamide;
N-((6-tert-butyl-2-(4-(dimethylamino)-4-phenylpiperidin-1-yl)pyridin-3-yl)methyl)-2-(3-fluoro-4-(methylsulfonamido)phenyl)propanamide;
N-((2-(4-benzyl-4-(dimethylamino)piperidin-1-yl)-6-(trifluoromethyl)pyridin-3-yl)methyl)-2-(3-fluoro-4-(methylsulfonamido)phenyl)propanamide;
N-((2-(4-benzyl-4-(dimethylamino)piperidin-1-yl)-6-tert-butylpyridin-3-yl)methyl)-2-(3-fluoro-4-(methylsulfonamido)phenyl)propanamide;
N-(2-(4-benzyl-4-(dimethylamino)piperidin-1-yl)-4-(trifluoromethyl)benzyl)-2-(3-fluoro-4-(methylsulfonamido)phenyl)propanamide;
N-(2-(4-benzyl-4-(dimethylamino)piperidin-1-yl)-4-(trifluoromethyl)benzyl)-2-(3-methyl-4-(methylsulfonamido)phenyl)propanamide;
N-((2-(4-(dimethylamino)-4-(3-fluorobenzyl)piperidin-1-yl)-6-(trifluoromethyl)pyridin-3-yl)methyl)-2-(3-fluoro-4-(methylsulfonamido)phenyl)propanamide;
N-(2-(4-(dimethylamino)-4-(3-fluorobenzyl)piperidin-1-yl)-4-(trifluoromethyl)benzyl)-2-(3-fluoro-4-(methylsulfonamido)phenyl)propanamide; and
N-(2-(4-(dimethylamino)-4-(3-fluorobenzyl)piperidin-1-yl)-4-(trifluoromethyl)benzyl)-2-(3-methyl-4-(methylsulfonamido)phenyl)propanamide;
   in the form of one of its individual stereoisomers, in particular enantiomers or diastereomers, its racemates or in the form of a mixture of stereoisomers, in particular enantiomers and/or diastereomers, in any desired mixture ratio, or respectively in the form of corresponding salts, or respectively in the form of corresponding solvates.

Pharmaceutical compositions that are particularly preferred comprise at least one compound selected from the group consisting of:
N-((2-(4-(dimethylamino)-4-(thiophen-2-yl)piperidin-1-yl)-6-(trifluoromethyl)pyridin-3-yl)methyl)-2-(3-fluoro-4-(methylsulfonamido)phenyl)propanamide; and
N-(2-(4-(dimethylamino)-4-(thiophen-2-yl)piperidin-1-yl)-4-(trifluoromethyl)benzyl)-2-(3-fluoro-4-(methylsulfonamido)phenyl)propanamide;
   in the form of one of its individual stereoisomers, in particular enantiomers or diastereomers, its racemates or in the form of a mixture of stereoisomers, in particular enantiomers and/or diastereomers, in any desired mixture ratio, or respectively in the form of corresponding salts, or respectively in the form of corresponding solvates.

Pharmaceutical compositions containing at least one compound corresponding to formula (I), (II), (III) or (IV) can also be used to produce a drug for the treatment of overactive bladder syndrome, coughing, asthma, COPD and/or diabetes. Moreover, these pharmaceutical compositions can contain at least one compound selected from the group consisting of
N-((2-(4-(dimethylamino)-4-phenylpiperidin-1-yl)-6-(trifluoromethyl)pyridin-3-yl)methyl)-2-(3-fluoro-4-(methylsulfonamido)phenyl)propanamide;
N-((6-tert-butyl-2-(4-(dimethylamino)-4-phenylpiperidin-1-yl)pyridin-3-yl)methyl)-2-(3-fluoro-4-(methylsulfonamido)phenyl)propanamide;
N-((2-(4-benzyl-4-(dimethylamino)piperidin-1-yl)-6-(trifluoromethyl)pyridin-3-yl)methyl)-2-(3-fluoro-4-(methylsulfonamido)phenyl)propanamide;
N-((2-(4-benzyl-4-(dimethylamino)piperidin-1-yl)-6-tert-butylpyridin-3-yl)methyl)-2-(3-fluoro-4-(methylsulfonamido)phenyl)propanamide;
N-(2-(4-benzyl-4-(dimethylamino)piperidin-1-yl)-4-(trifluoromethyl)benzyl)-2-(3-fluoro-4-(methylsulfonamido)phenyl)propanamide;
N-(2-(4-benzyl-4-(dimethylamino)piperidin-1-yl)-4-(trifluoromethyl)benzyl)-2-(3-methyl-4-(methylsulfonamido)phenyl)propanamide;
N-((2-(4-(dimethylamino)-4-(3-fluorobenzyl)piperidin-1-yl)-6-(trifluoromethyl)pyridin-3-yl)methyl)-2-(3-fluoro-4-(methylsulfonamido)phenyl)propanamide;
N-(2-(4-(dimethylamino)-4-(3-fluorobenzyl)piperidin-1-yl)-4-(trifluoromethyl)benzyl)-2-(3-fluoro-4-(methylsulfonamido)phenyl)propanamide; and
N-(2-(4-(dimethylamino)-4-(3-fluorobenzyl)piperidin-1-yl)-4-(trifluoromethyl)benzyl)-2-(3-methyl-4-(methylsulfonamido)phenyl)propanamide;
   in the form of one of its individual stereoisomers, in particular enantiomers or diastereomers, its racemates or in the form of a mixture of stereoisomers, in particular enantiomers and/or diastereomers, in any desired mixture ratio, or respectively in the form of corresponding salts, or respectively in the form of corresponding solvates.

In a preferred embodiment pharmaceutical compositions that contain at least one compound corresponding to formula (V) can additionally be used to produce a drug for the treatment of overactive bladder syndrome, coughing, asthma, COPD and/or diabetes. Moreover, these pharmaceutical compositions can contain at least one compound selected from the group consisting of:
N-((2-(4-(dimethylamino)-4-(thiophen-2-yl)piperidin-1-yl)-6-(trifluoromethyl)pyridin-3-yl)methyl)-2-(3-fluoro-4-(methylsulfonamido)phenyl)propanamide; and
N-(2-(4-(dimethylamino)-4-(thiophen-2-yl)piperidin-1-yl)-4-(trifluoromethyl)benzyl)-2-(3-fluoro-4-(methylsulfonamido)phenyl)propanamide;
   in the form of one of its individual stereoisomers, in particular enantiomers or diastereomers, its racemates or in the form of a mixture of stereoisomers, in particular enantiomers and/or diastereomers, in any desired mixture ratio, or respectively in the form of corresponding salts, or respectively in the form of corresponding solvates.

The compounds with a dual mode of action can be used in the pharmaceutical composition according to the invention in the form of a physiologically compatible acid addition salt. Physiologically compatible acid addition salts are understood to be pharmaceutically compatible salts according to the invention that are preferably selected from the salts of hydrochloric acid, hydrobromic acid, sulfuric acid, phosphoric acid, methanesulfonic acid, acetic acid, fumaric acid, maleic acid, succinic acid, lactic acid, citric acid, tartaric acid and maleic acid. Mixtures of the aforementioned acids can possibly also be used for the production of the salts. The salts of the compounds with dual mode of action selected from the group consisting of hydrochloride, hydrobromide, sulfate, phosphate, fumarate and methanesulfonate are preferred according to the invention. The salts selected from the group consisting of hydrochloride, hydrobromide and fumarate are particularly preferred, wherein hydrochloride is considered the most significant according to the invention. The compounds with dual mode of action according to the invention may possibly be present in the form of their hydrate.

If the compounds with dual mode of action carry acid groups such as e.g. —COOH, —SO₃H and/or —PO₃H₂ groups, these can also be present in the pharmaceutical composition according to the invention as physiologically compatible salts that are formed by the addition of a suitable base. The following can preferably be used as bases: hydroxides, hydrogencarbonates and/or carbonates of alkali and/or earth alkali metals, preferably sodium hydroxide, potassium hydroxide, sodium hydrogencarbonate, potassium hydrogencarbonate, calcium hydrogencarbonate, magnesium hydrogencarbonate, sodium carbonate, potassium carbonate, calcium carbonate and/or magnesium carbonate. Moreover, unsubstituted and/or substituted amines can also be used as bases. Alkali, earth alkali or ammonium salts of the VR1 receptor ligands are preferred. Mixtures of the aforementioned bases can possibly also be used to produce the salts.

The affinity of the compounds with a dual mode of action to μ-opioid receptors, determined based on the Kᵢ value and preferably measured by in vitro tests, preferably amounts to ≦5.0 μM, more preferred ≦1.0 μM, further preferred ≦100 nM, most preferred ≦10 nM and in particular ≦1.0 nM. At the same time, the affinity of the compounds with dual mode of action to VR1 receptors, determined based on the Kᵢ value and preferably measured by in vitro tests, preferably amounts to ≦5.0 μM, more preferred ≦1.0 μM, further preferred ≦100 nM, most preferred ≦10 nM and in particular ≦1.0 nM. Instructions for conducting the corresponding in vitro tests for determining the μ-opioid receptor and VR1 receptor affinity are known to persons skilled in the art from the technical literature.

In one preferred embodiment of the composition according to the invention, the compounds with a dual mode of action are used in combination with further compounds that have affinity to the μ-opioid receptor and/or VR1 receptor.

The compounds of the pharmaceutical composition according to the invention can be used in particular for the production of a drug for the treatment of pain. In this case, persons skilled in the art know that the effect on VR1 receptors and on μ-opioid receptors can be achieved by a single compound, which combines both modes of action in itself (dual mode of action), or by at least two compounds.

In this case, the drug according to the invention and the pharmaceutical composition according to the invention can preferably be used for the treatment of acute and chronic pain selected from the group consisting of neuropathic pain, post-zoster neuralgia, pain in patients with increased addiction potential, pain in patients with opioid-induced hyperalgesia, pain in patients with tolerance development with respect to opioid analgesics, pain in patients with allodynia, diabetic neuropathy, pain in patients over 60 years of age and during narcosis, post-operative pain, pain in patients suffering from a psychological disorder. Moreover, the combination according to the invention comprising VR1 receptor ligands and μ-opioid receptor ligands can be used for narcosis and for analgesia during narcosis. In this case, the combination according to the invention is especially suitable for treating patients over 60 years of age.

The drug according to the invention and the pharmaceutical composition according to the invention can also preferably be used for the treatment of overactive bladder syndrome, coughing, asthma, COPD and/or diabetes.

The drug according to the invention and the pharmaceutical composition according to the invention can preferably be used for the treatment of acute and chronic pain selected from the group consisting of neuropathic pain, pain in patients with opioid-induced hyperalgesia, pain in patients with tolerance development with respect to opioid analgesics and pain in patients with allodynia. The administration for treatment of neuropathic pain and pain in patients with allodynia is particularly preferred.

The amount of active substance to be administered to patients varies depending on the weight of the patient, the type of application, the indication and the degree of severity of the illness.

Besides at least one compound with the properties according to the invention or a combination according to the invention, the drug according to the invention and the pharmaceutical composition according to the invention preferably possibly contains suitable additives and/or adjuvants, including support materials, fillers, solvents, diluting agents, colouring agents and/or binders, and can be administered as liquid drug forms in the form of injectable solutions, drops or juices, as semisolid drug forms in the form of granules, tablets, pellets, patches, capsules, plasters or aerosols. The selection of adjuvants etc. as well as the amounts thereof to be used are dependent on whether application of the drug is to be by oral, peroral, parenteral, intravenous, intraperitoneal, intradermal, intramuscular, intranasal, buccal administration, rectally or locally, e.g. onto the skin, via the mucous membranes or into the eyes.

Preparations in the form of tablets, coated tablets, capsules, granules, drops, juices and syrups are suitable for oral application, and solutions, suspensions, easily reconstituted dry preparations as well as sprays are suitable for parenteral, topical and inhalatory application. The pharmacologically active compounds of the combination according to the invention in a depot, in dissolved form or in a plaster, possibly with the addition of skin penetration promoters are suitable preparations for percutaneous application. Preparation forms to be applied orally or percutaneously can release the compounds with the properties according to the invention or a combination according to the invention in a delayed manner. In principle, other additional active substances known to the person skilled in the art can be added to the drugs according to the invention and the pharmaceutical compositions according to the invention.

### BRIEF DESCRIPTION OF THE DRAWINGS

FIG. 1 shows antinociception in the tail-flick test in rats represented based on the increase in pain latency.

FIG. 2 shows the mechanical analgesic efficacy tested using rats after spinal nerve ligature (ipsi-lateral rear paw).

FIG. 3 shows the mechanical analgesic efficacy tested using rats after spinal nerve ligature.

### EXAMPLES

The following examples serve to explain the invention in further detail without restricting the overall scope of the invention.

### Example 1

The following experiments of Example 1 show the difference between the analgesic efficacy of a combination, which contains the VR1 receptor antagonist AMG 9810 (Gavva et al., JPET 2005, 313, 474-484) and the μ-opioid agonist morphine, compared to the application of morphine alone. In addition, the following experiments of Example 1 show the difference between the analgesic efficacy of a combination, which contains the VR1 receptor antagonist AMG-517 (Gavva et al., JPET 2007, 323, 128-137; Doherty et al., J. Med. Chem. 2007, 50, 3515-3527) and the μ-opioid agonist oxycodone, compared to the application of oxycodone alone. The experiments comprise the determination of the analgesic efficacy in the case of acute pain (tail-flick test in rats) and in the case of chronic neuropathic pain (Chung model after spinal nerve ligature in rats).

The following compounds were tested:

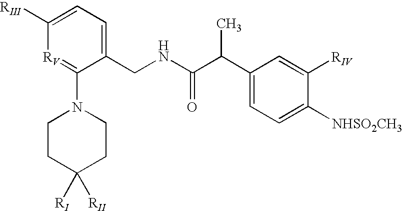

| Compound | R_{I} | R_{II} | R_{III} | R_{IV} | R_{V} |
|---|---|---|---|---|---|
| [1] | —N(CH₃)₂ | 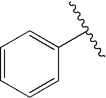 | —CF₃ | —F | N |
| [2] | —N(CH₃)₂ | 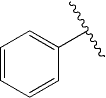 | —C(CH₃)₃ | —F | N |
| [3] | —N(CH₃)₂ | 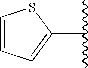 | —CF₃ | —F | N |
| [4] | —N(CH₃)₂ | 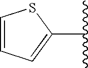 | —CF₃ | —F | CH |
| [5] | —N(CH₃)₂ | 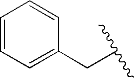 | —CF₃ | —F | N |
| [6] | —N(CH₃)₂ | 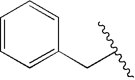 | —C(CH₃)₃ | —F | N |
| [7] | —N(CH₃)₂ | 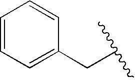 | —CF₃ | —F | CH |
| [8] | —N(CH₃)₂ | 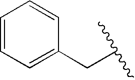 | —CF₃ | —CH₃ | CH |
| [9] | —N(CH₃)₂ | 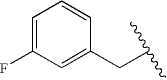 | —CF₃ | —F | N |
| [10] | —N(CH₃)₂ | 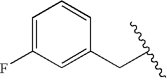 | —CF₃ | —F | CH |
| [11] | —N(CH₃)₂ | 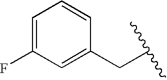 | —CF₃ | —CH₃ | CH |

### Test Animals

Sprague Dawley rats (140-160 g body weight) were used for the following animal models. Sprague Dawley rats (140-160 g body weight) from a commercial breeder (Janvier, Genest St. Isle, France) were used. The animals were kept under standard conditions: light-dark rhythm (06:00 to 18:00 h light, 18:00 to 06:00 h dark), room temperature 19-23° C., relative air humidity 45-65%, air exchange 19 times per hour, air movement <0.2 m/s, free choice of tap water and standard feed, Macrolon Type 4 cages with 5 animals per cage. The solutions of the test substances and/or vehicles (10% DBSO, 5% Cremophor EL, 5% glucose) were administered intravenously.

### Statistical Analysis

The data were analysed by ANOVA analysis (two-factor analysis of variance) with repeated measurement. On determination of a significant effect of treatment a post hoc analysis with Bonferronic correction was conducted. The results were graded as statistically significant when p<0.05. Each group consisted of n=10 animals.

### Experiment 1

### Analgesia Study in the Tail-Flick Test in Rats

The analgesic efficacy of the test compounds was examined in the hot beam (tail flick) test in rats using the method of D'Amour and Smith (J. Pharm. Exp. Ther. 1941, 72, 74-79). The time from switching on the lamp (8V/50 W) to the sudden flicking away of the tail from the hot beam (pain latency) was measured by means of a semiautomatic apparatus (tail-flick Analgesiemeter Type 50/08/1.bc, Labtec, Dr. Hess, Germany). The lamp intensity was adjusted before conducting the experiments so that the time from switching on the lamp to the sudden flicking away of the tail (pain latency) amounted to 3-5 seconds in untreated animals. The lamp was automatically switched off after 12 seconds to avoid tissue damage to the rat's tail. The pain latency was measured 10, 20, 40 and 60 min after intravenous administration. The analgesic effect was determined as increase in pain latency (% MPE) according to the following formula:

% MPE=[(T₁−T₀)/(T₂−T₀)]×100.

In this case, T₀ is the latency time before and T₁ the latency time after substance application, T₂ is the maximum exposure time (12 sec).
Results of the analgesia study in the tail-flick test in rats:

TABLE 1
Antinociceptive efficacy in the tail-flick test shown based on the increase in pain
latency % MPE (mean ± SEM)

| | % MPE | | | | | | | |
|---|---|---|---|---|---|---|---|---|
| | 10 min | | 20 min | | 40 min | | 60 min | |
| Treatment (mg/kg) i.v. | Mean | SEM | Mean | SEM | Mean | SEM | Mean | SEM |
| Vehicle | 0.76 | 4.24 | −6.08 | 1.95 | −4.6 | 2.73 | −11.42 | 3.26 |
| Morphine (0.4) | 26.51 | 10.74 | 23.62 | 12.81 | 14.1 | 10.87 | 4.23 | 7.16 |
| Morphine (0.8) | 45.31 | 13.7 | 45.02 | 13.79 | 25.86 | 10.92 | 14.62 | 11.12 |
| AMG-9810 (10) | 26.36 | 7.86 | 11.44 | 6.66 | 4.38 | 5.71 | −4.06 | 3.55 |
| Morphine (0.4) + | 64.72 | 13.96 | 60.57 | 12.83 | 42.18 | 10.91 | 26.31 | 10.08 |
| AMG-9810 (10) | | | | | | | | |

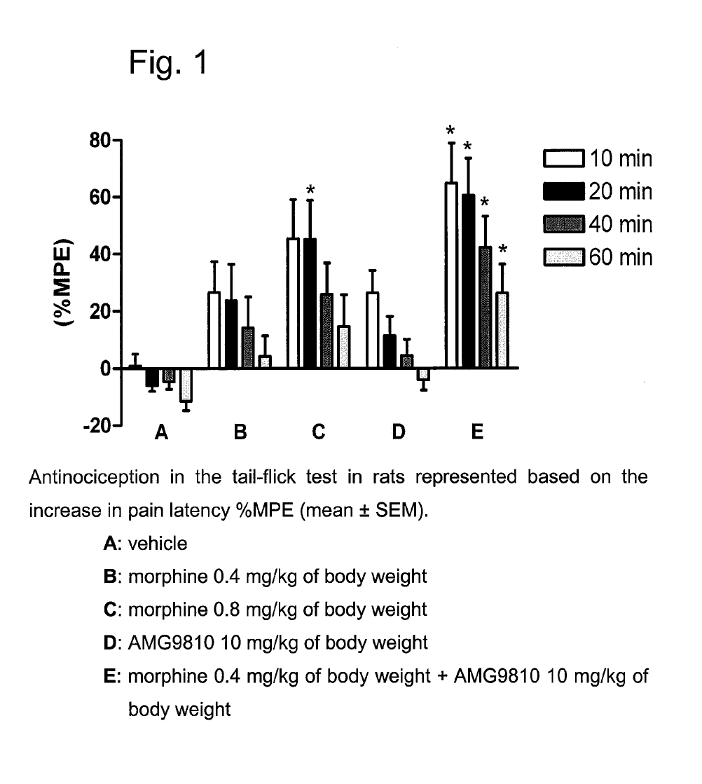

Conclusion: the combination of two threshold doses of morphine results in an additive antinociceptive efficacy. The combination of one threshold dose of morphine and one threshold dose of AMG-9810 results in a superadditive antinociceptive efficacy. The above results are also reproduced in FIG. 1.

### Experiment 2

### Mono-Neuropathic Pain after Spinal Nerve Ligature (Chung Model)

Under pentobarbital narcosis (Narcoren®, 60 m/kg i.p., Merial GmbH, Hallbergmoos, Germany), the L5, L6 spinal nerves were firmly bound. The left L5 and L6 spinal nerves were exposed by removing a piece of paravertebral muscle and a portion of the left spinal process of the L5 lumbar vertebral body. The L5 and L6 spinal nerves were carefully isolated and bound with a firm ligature (NC silk, black, USP 5/0, metric 1, Braun Melsungen AG, Melsungen, Germany) (Kim and Chung, An experimental model for peripheral neuropathy produced by segmental spinal nerve ligature in the rat, Pain, 50, 1992, 355-363). After haemostasis was determined, the muscle and adjacent tissue were sutured and the wound closed by metal clamps. After a one-week recovery period the animals were placed in cages with a wire base closed to the top by a plastic hood for measurement of the mechanical allodynia. The animals were kept in this cage until their explorative behaviour had decreased.

The threshold for the tactile allodynia was measured by means of an electronic von Frey anaesthesiometer (Somedic AB, Malmö, Sweden). The animals were randomly divided into groups of 10 animals. Each group received an individual dose of test substance or vehicle and the measurements were conducted 0.5 h before application and at various times after intravenous administration. Both the ipsi- and the contra-lateral rear paw were tested. The median of five stimulations gave a measured data point. The data were determined as % maximum possible effect (% MPE) from the pre-tests of the Chung individual animals (=0% MPE) and the test values of an independent sham control group (=100% MPE). The effect of the test substance and the vehicle was calculated for each measurement time (i.e. 0.5, 1 and 3 h after application) as inter-individual % MPE value. The anti-allodynic efficacy is defined as the increase in the threshold value for pull-away of the rear paw without influence on the threshold for pull-away of the contra-lateral rear paw. The corresponding threshold values are given in gram (mean±SEM).

### Test Results: Chung Model:

TABLE 2
Mechanical analgesic efficacy tested using rats after spinal nerve
ligature shown based on the pull-away threshold values of the ipsi- and
contra-lateral rear paw given in gram (mean ± SEM).

| | G | | | | | | | |
|---|---|---|---|---|---|---|---|---|
| | Control | | 30 min | | 60 min | | 180 min | |
| Treatment | ipsi | | ipsi | | ipsi | | ipsi | |
| (mg/kg) i.v. | Mean | SEM | Mean | SEM | Mean | SEM | Mean | SEM |
| Sham vehicle | 65.45 | 4.8 | 56.24 | 2.5 | 55.76 | 2.4 | 56.3 | 2.2 |
| Vehicle | 22.84 | 1.6 | 26.42 | 0.9 | 25.72 | 1.3 | 26.64 | 1.2 |
| Morphine (1.0) | 23.63 | 1.4 | 32.13 | 2.7 | 27.65 | 1.5 | 30.67 | 2.3 |
| Morphine (2.0) | 24.37 | 1.2 | 48.44 | 3.4 | 44.87 | 4.5 | 39.19 | 3.6 |
| AMG-9810 | 23.23 | 0.9 | 32.27 | 4.3 | 31.18 | 3.9 | 26.94 | 1.6 |
| (2.15) | | | | | | | | |
| Morphine | 22.63 | 1.44 | 42.47 | 3.74 | 44.35 | 2.69 | 35.21 | 3.15 |
| (1.0) + | | | | | | | | |
| AMG-9810 | | | | | | | | |
| (2.15) | | | | | | | | |
| | G | | | | | | | |
| | Control | | 30 min | | 60 min | | 180 min | |
| Treatment | contra | | contra | | contra | | contra | |
| (mg/kg) i.v. | Mean | SEM | Mean | SEM | Mean | SEM | Mean | SEM |
| Sham vehicle | 63.71 | 3.2 | 54.59 | 2.8 | 55.25 | 2.8 | 48.96 | 3.4 |
| Vehicle | 61.84 | 2.9 | 59.31 | 2.5 | 59.95 | 2.3 | 58.66 | 1.7 |
| Morphine (1.0) | 56.81 | 1.9 | 67.87 | 3.4 | 60.95 | 2.6 | 54.63 | 2.2 |
| Morphine (2.0) | 58.39 | 1.6 | 82.83 | 5.9 | 74.68 | 2.8 | 59.93 | 2.5 |
| AMG-9810 | 57.79 | 2.2 | 55.91 | 2.1 | 52.4 | 1.8 | 57.38 | 3.5 |
| (2.15) | | | | | | | | |
| Morphine | 59.66 | 2.0 | 61.57 | 2.1 | 55.58 | 2.4 | 58.75 | 2.7 |
| (1.0) + | | | | | | | | |
| AMG-9810 | | | | | | | | |
| (2.15) | | | | | | | | |
| | G | | | | | | | |
| | Control | | 30 min | | 60 min | | 180 min | |
| Treatment | ipsi | | ipsi | | ipsi | | ipsi | |
| (mg/kg) i.v. | Mean | SEM | Mean | SEM | Mean | SEM | Mean | SEM |
| Sham vehicle | 53.5 | 1.2 | 54.5 | 0.8 | 53.8 | 1.4 | 53.6 | 1.5 |
| Vehicle | 20.7 | 1.2 | 19.7 | 1.2 | 20.9 | 1.1 | 21.1 | 0.9 |
| Oxycodone | 21.0 | 0.7 | 38.2 | 1.3 | 28.0 | 1.1 | 21.9 | 0.7 |
| (2.5) | | | | | | | | |
| Oxycodone (5) | 22.8 | 1.0 | 41.9 | 1.9 | 32.1 | 1.8 | 22.0 | 1.0 |
| AMG 517 (2.5) | 20.6 | 1.4 | 29.2 | 0.9 | 21.1 | 2.2 | 20.7 | 0.9 |
| Oxy (2.5) + | 20.6 | 0.8 | 34.9 | 1.2 | 27.8 | 1.5 | 22.6 | 0.8 |
| (AMG 2.5) | | | | | | | | |
| | G | | | | | | | |
| | Control | | 30 min | | 60 min | | 180 min | |
| Treatment | contra | | contra | | contra | | contra | |
| (mg/kg) i.v. | Mean | SEM | Mean | SEM | Mean | SEM | Mean | SEM |
| Sham vehicle | 53.9 | 1.3 | 53.4 | 0.8 | 53.6 | 1.6 | 57.3 | 1.0 |
| Vehicle | 51.9 | 1.0 | 52.4 | 1.3 | 52.5 | 0.8 | 52.7 | 1.5 |
| Oxycodone | 54.0 | 0.9 | 67.8 | 3.2 | 53.1 | 1.6 | 52.5 | 1.4 |
| (2.5) | | | | | | | | |
| Oxycodone (5) | 52.6 | 1.1 | 70.0 | 1.5 | 56.4 | 2.4 | 51.4 | 1.1 |
| AMG 517 (2.5) | 49.9 | 1.4 | 50.9 | 1.3 | 49.6 | 1.1 | 51.7 | 0.7 |
| Oxy (2.5) + | 54.7 | 1.7 | 59.2 | 2.4 | 54.3 | 1.7 | 53.4 | 1.2 |
| AMG (2.5) | | | | | | | | |

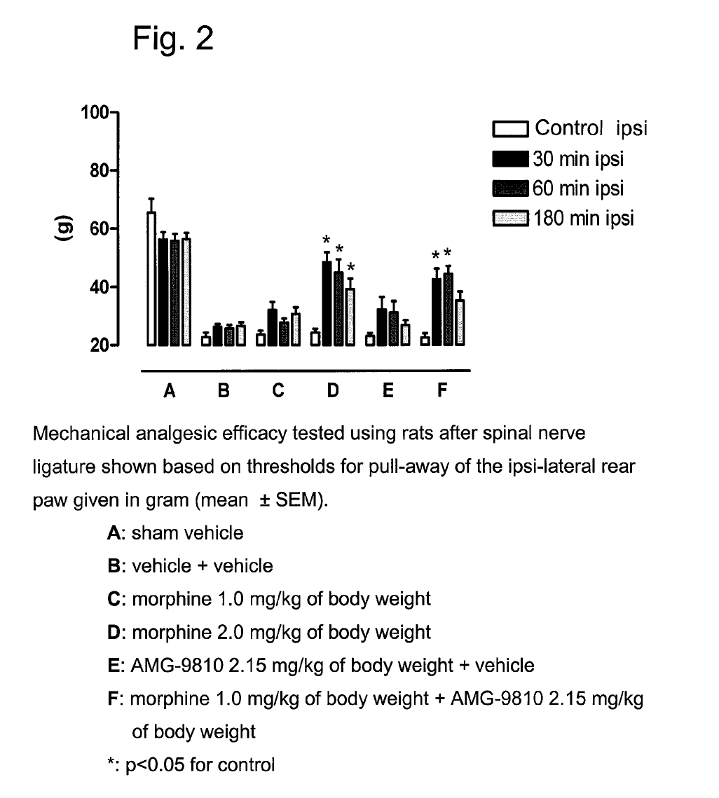

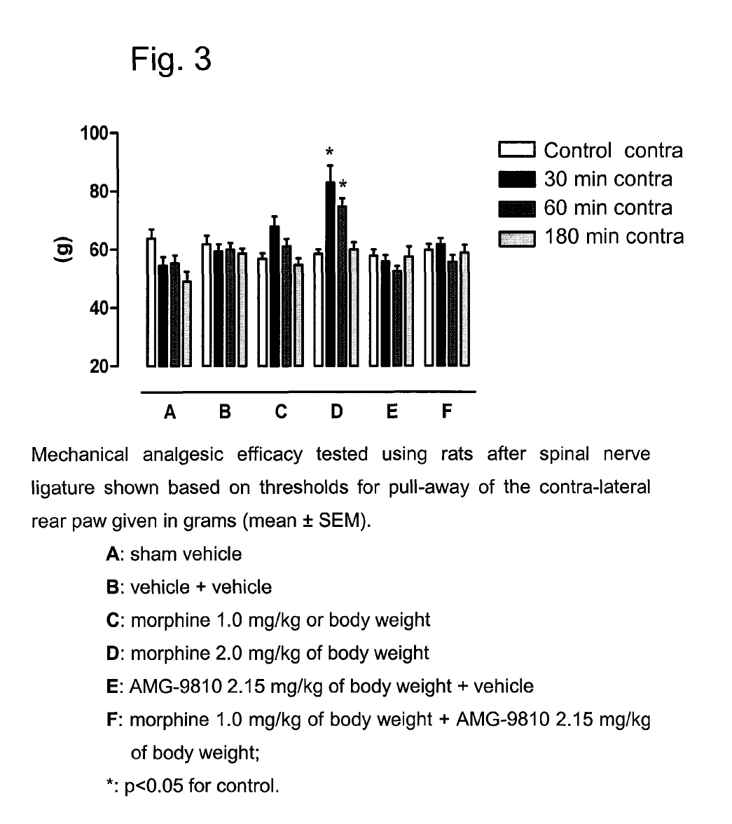

Conclusion: the combination of two threshold doses of morphine results in an additive antinociceptive efficacy, indicated by the significant increase in pull away of both the ipsi- and the contra-lateral rear paw. The combination of one threshold dose of morphine and one threshold dose of AMG-9810 leads to an anti-allodynic efficacy, indicated by the significant increase in pull away of the ipsi-lateral and not the contra-lateral rear paw. The corresponding results are also reproduced in FIGS. 2 and 3.
Conclusion: the combination of two threshold doses of oxycodone results in an additive antinociceptive efficacy, indicated by the significant increase in pull away of both the ipsi- and the contra-lateral rear paw. The combination of one threshold dose of oxycodone and one threshold dose of AMG-517 leads to an anti-allodynic efficacy, indicated by the significant increase in pull away of the ipsi-lateral and not the contra-lateral rear paw.

### Example 2

The following experiments of Example 2 relate to binding studies and the analgesic efficacy of the compounds with dual mode of action according to the invention. The experiments comprise the determination of the (f)Kᵢ value at the TPRV1 receptor, the Kᵢ value at the μ-opioid receptor and the hypothermia assay in mice. The tail-flick test for the compounds [1], [4], [7] and [10] according to the invention was conducted in a similar manner to the above-described method (cf. Example 1, experiment 1).

### Experiment 3

### Hypothermia Assay in Mice

The hypothermia assay was conducted on male NMRI mice (weight 25-35 gram). The animals were kept in standardised conditions: light/dark rhythm (6:00 to 18:00 h light phase, 18:00 to 6:00 h dark phase), room temperature 19-22° C., relative air humidity 35-70%, air exchange 15 times per hour, air movement <0.2 m/sec. The animals received standard feed (ssniff R/M-Haltung, ssniff Spezialdiäten GmbH, Soest, Germany) and tap water. Water and feed were withdrawn during the test. All the animals were used only once in the test. The animals had an acclimatisation phase of at least 5 days. The acute application of capsaicin (VR-1 agonist) causes a drop in core body temperature in rats and mice via a stimulation of heat sensors. Only specific active VR-1 receptor antagonists can antagonise capsaicin-induced hypothermia. In contrast, hypothermia induced by morphine is not antagonised by VR-1 antagonists. This model is therefore suitable for identifying substances with VR-1 antagonist properties via the effect on the body temperature.

A digital thermometer (Thermalert TH-5, physitemp, Clifton N.J., USA) is used for measuring the core body temperature. The measurement sensor is inserted into the rectum of the animals in this case. As an individual base value the body temperature is measured twice in each animal at an interval of approximately half an hour. A group of animals (n=6 to 10) then received an intraperitoneal (i.p.) application of capsaicin of 3 mg/kg and vehicle (control group). Another group of animals received the substance to be tested (i.v. or p.o.) and additionally capsaicin (3 mg/kg) i.p. The test substance was applied i.v. 10 min or p.o. 15 minutes before the application of capsaicin. Then, the body temperature was measured 7.5, 15 and 30 min after capsaicin application (i.v.+i.p.) or 15, 30, 60, 90 and 120 min after capsaicin application (p.o.+i.p.). In addition, one animal group was treated only with test substance and one group was treated only with vehicle. The antagonist effect is calculated as a percentage reduction in the capsaicin-induced hypothermia.

### Experiment 4

### Agonist-Induced Stimulation of the [³⁵]GTPyS Binding

This assay was conducted to determine the potency (IC₅₀) of the compounds according to the invention at the μ-opioid receptor. The test procedure was based on the methods described in specialist literature (Gillen et al. Naunyn Schmiedeberg's Arch. Pharmacol. 2000, 362, 116-121). The test was conducted as a homogenous SPA assay (scintillation proximity assay) in microtiter luminescence plates (Costar, Cambridge Mass., USA). Each individual well contained 1.5 mg of WGA-coated SPA beads (GE Healthcare UK Ltd., Buckinghamshire, UK). To determine the agonist activity of the compounds according to the invention on the human recombinant μ-opioid receptor, cell membranes of CHO-K1 cells with human recombinant μ-opioid receptors (10 μg membrane protein/assay) were incubated for 45 min at room temperature with 0.4 nmol/L of [³⁵S]GTPyS (GE Healthcare UK Ltd., Buckinghamshire, UK) and dilutions of the test compounds in assay buffer (20 mM HEPES pH 7.4, 100 mM NaCl, 10 mM MgCl₂, 1.0 mM EDTA, 1.0 mM dithiothreitol, 1.28 NaN₃ and 10 μM GDP).

The test compounds were diluted using the solvent N-methylpyrrolidone to assure a reliable dilution. The final concentration of the solvent in the assay buffer amounted to 0.5%. The microtiter plates were then centrifuged for 10 min at 830 g in an Omnifuge 2. ORS microtiter plate centrifuge (Kendro Laboratory Products, Langensebold, Germany) to settle the SPA beads. The microtiter plates were sealed (Top Seals®) and the linked radioactivity was determined after 15 min by means of a 1450 Microbeta Trilux (PerkinElmer Life Sciences, Inc., Boston, Mass., USA). The basal binding activity (UBS_{obs}) was determined based on 12 non-stimulated incubations and set at 100%. All other incubations were conducted in duplicate. The potency and efficacy of the total binding (TB_{obs}) of [³⁵S]GTPyS in the test series stimulated by the enkephalin DAMGO or the test compounds was determined (% binding in relation to the basal binding activity). The potency (EC₅₀) and the maximum achievable increase (B1_{calc}) of the [³⁵S]GTPyS binding above the basal binding (% UBS_{calc}) was determined by means of non-linear regression analysis with the software GraphPad Prism for each individual concentration series (GraphPad Software Inc., San Diego, USA).

To determine the relative efficacy compared to the μ-opioid-specific reference enkephalin DAMGO, the calculated maximum increase in the [³⁵S]GTPyS binding (B1_{calc/DAMGO}) to be achieved above the calculated basal binding (UBS_{calc/DAMGO}) as a result of DAMGO was equated with 100% relative efficacy. The calculated maximum increase in the [³⁵S]GTPyS binding (B1_{calc/test cpd.}) to be achieved as a result of the test compound [%] was converted into the relative efficacy of DAMGO. The mean of the relative efficacy of a test compound compared to DAMGO was determined based on three independent experiments, each of which studied DAMGO and the test compound in parallel experiments.

### Experiment 5

### FLIPR Assay

The agonist or antagonist effect of substances can be determined at the vanilloid receptor 1 (VR1/TRPV1) of the human and rat species with the assay described below. Accordingly, the Ca²⁺ influx through the channel is quantified by means of a Ca²⁺-sensitive dye (Fluo-4, Molecular Probes Europe BV, Leiden, The Netherlands) in the fluorescent imaging plate reader (FLIPR, Molecular Devices, Sunnyvale, USA).

### Method:

Chinese hamster ovary cells (CHO K1 cells, ECACC, England) are stably transfected with human or rat VR1 cDNA. For functional studies these cells are plated onto poly-D-lysine-coated black 96-hole plates with a clear base (BD Biosciences, Heidelberg, Germany) in a density of 25 000 cells/cavity. The cells are incubated overnight at 37° C. with 5% CO₂ in culture medium (nutrient mixture Ham's F12, Gibco Invitrogen GmbH, Karlsruhe, Germany) with 10% vol. FBS (foetal bovine serum, Gibco Invitrogen GmbH, Karlsruhe, Germany) and 18 μg/ml of L-proline (Gibco Invitrogen GmbH, Karlsruhe, Germany). On the following day, the cells are laden with 2 μM Fluo-4 and 0.01% vol. Pluronic F127 (Molecular Probes Europe BV, Leiden, The Netherlands) in HBSS buffer (Hank's buffered saline solution, Gibco Invitrogen GmbH, Karlsruhe, Germany) for 30 min at 37° C. The plates are then washed 3 times in HBSS buffer and after further incubation for 15 minutes at room temperature are placed in the FLIPR for the Ca²⁺ measurement. In this case, the Ca²⁺-dependent fluorescence is measured before and after the addition of substances (λₑₓ=488 nm, λₑₘ=540 nm). Quantification occurs as a result of the measurement of the highest fluorescence intensity (FC, fluorescence counts) over time.

### FLIPR Capsaicin Assay:

The FLIPR protocol consists of 2 substance additions. Test substances (10 μM) are firstly pipetted onto the cells and the Ca²⁺ influx is compared to the control (capsaicin 10 μM). This gives the specification in % activation in relation to the Ca²⁺ signal after adding 10 μM of capsaicin. After 5 minutes incubation 100 nM of capsaicin are applied and the influx of Ca²⁺ likewise determined. Desensitising agonists and antagonists lead to a suppression of the Ca²⁺ influx. Inhibition values [%] are calculated in comparison to the maximum achievable inhibition with a saturating concentration of capsaicin or another reference antagonist. The test substance is added in different concentrations to determine the IC₅₀ and functional Ki value ((f)Ki). Triple determinations (n=3) are conducted and these are repeated in at least one further independent experiment (N>=2). A capsaicin dose action curve is generated to incorporate the thus determined EC₅₀ values into the associated (f)Ki determination. EC₅₀/IC₅₀ values are calculated using ‘Prism4’ software from GraphPad Software™ or with XLfit4™ (ID Business Solutions Ltd.). (f)Ki values are calculated in accordance with the modified Cheng-Prusoff equation (Cheng, Prusoff; Biochem. Pharmacol. 22, 3099-3108, 1973). Testing for non-specific inhibition of the fluorescence as a result of the substances is conducted by activating wild type CHO K1 cells incubated with the same test substances with ATP (10 μM final concentration).

### FLIPR pH Assay:

In the pH assay, HBSS that now contains 60 mM MES instead of capsaicin is applied to human VR1-transfected CHO K1 cells (adjusted pH value of the addition solution is 4.95). Percentage inhibition and IC₅₀ values are raised and evaluated as already described for the capsaicin test.

The test results for experiments 3, 4, 5 and the tail-flick assay for compounds [1], [4], [7] and [10] are summarized in the following Table 3:

TABLE 3

| | f(Kᵢ) | IC₅₀ | Kᵢ | Hypothermia | |
|---|---|---|---|---|---|
| | (VR1) | (pH) | (MOR) | Assay | Tail-Flick |
| Compound | [nM] | [nM] | [nM] | [% Inhibition] | [% MPE] |
| [1] | 31.3 | 806 | 46 | n.e.¹⁾ | 50%²⁾ |
| [4] | 5.0 | 776 | 61 | 4%¹⁾ | n.e.³⁾, 37%⁴⁾ |
| [7] | 2.8 | 434 | 24 | 59%¹⁾ | n.e.³⁾, 100%⁴⁾ |
| [10] | 1.4 | 137 | 79 | 29%¹⁾ | n.e.³⁾ |

MOR = μ-opioid receptor;
n.e. = not determined;
¹⁾dosage: 3 mg/kg p.o.;
²⁾dosage: 1 mg/kg p.o.;
³⁾dosage: 10 mg/kg p.o.;
⁴⁾dosage: 30 mg/kg i.v.

The test results for experiments 4 and 5 for compounds [2], [3], [5], [6], [8], [9] and [11] are summarized in the following Table 4:

TABLE 4

| | (f)Kᵢ (VR1) | IC₅₀ (pH) | Kᵢ (MOR) |
|---|---|---|---|
| Compound | [nM] | [nM] | [nM] |
| [2] | 3.7 | 368 | 350 |
| [3] | 10.9 | 335 | 119 |
| [5] | 6.3 | 111 | 715 |
| [6] | 2.6 | 1363 | 3135 |
| [8] | 2.5 | n.e.¹⁾ | 22 |
| [9] | 2.4 | 145 | 2080 |
| [11] | 1.6 | n.e.²⁾ | 50 |

MOR = μ-opioid receptor;
¹⁾IC₅₀ not determined; the % inhibition was: 58% at 10 μM, 36% at 5 μM and 19% at 1.0 μM;
²⁾IC₅₀ not determined; the % inhibition was: 38% at 10 μM and 35% at 5.0 μM.

The foregoing description and examples have been set forth merely to illustrate the invention and are not intended to be limiting. Since modifications of the described embodiments incorporating the spirit and substance of the invention may occur to persons skilled in the art, the invention should be construed broadly to include all variations within the scope of the appended claims and equivalents thereof.

1. A method of treating pain in a subject, said method comprising administering to said subject a pharmaceutically effective amount of:
i) a combination comprising at least one compound that has affinity to the μ-opioid receptor and at least one compound that has affinity to the VR1 receptor, or
ii) at least one compound that has affinity to the μ-opioid receptor and the VR1 receptor;
wherein the μ-opioid receptor affinity expressed in terms of human Kᵢ value is ≦5.0 μM, and the VR1 receptor affinity expressed in terms of human Kᵢ value is ≦5.0 μM.

2. A method as claimed in claim 1, wherein a combination comprising a compound with affinity to the μ-opioid receptor and a compound with affinity to the VR1 receptor is used.

3. A method as claimed in claim 1, wherein a compound that has affinity to the μ-opioid receptor and to the VR1 receptor is used.

4. A method as claimed in claim 3, wherein said compound corresponds to formula I: 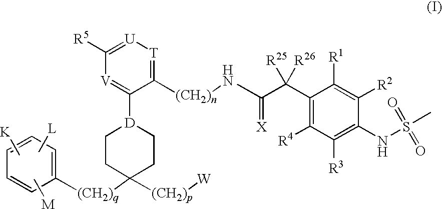
wherein
D is CH or N;
W stands for —CN, —NR³⁴R³⁵, —C(═O)—R³⁶ or —C(═O)—OR³⁷;
X stands for O, S or N—C≡N;
n stands for 0, 1, 2, 3 or 4;
p is 0, 1, 2 or 3;
q is 0, 1, 2 or 3;
T stands for C—R⁶ and U for C—R⁷ and V for N;
or
T stands for C—R⁶ and U for N and V for C—R⁹;
or
T stands for N and U for C—R⁷ and V for C—R⁹;
or
T stands for N and U for N and V for C—R⁹;
or
T stands for N and U for C—R⁷ and V for N;
or
T stands for C—R⁶ and U for N and V for N;
or
T stands for C—R⁶ and U for C—R⁷ and V for C—R⁹;
R¹, R², R³ and R⁴ each independently stand for H; F; Cl; Br; I; —SF₅; —NO₂; —CF₃; —CN; —NH₂; —OH; —SH; —C(═O)—NH₂; —S(═O)₂—NH₂; —C(═O)—NH—OH; —C(═O)—OH; —C(═O)—H; —S(═O)₂—OH; —NHR¹¹; —NR¹²R¹³; —OR¹⁴; —SR¹⁵; —C(═O)—NHR¹⁶; —C(═O)—NR¹⁷R¹⁸; —S(═O)₂—NHR¹⁹; —S(═O)₂—NR²⁰R²¹; —C(═O)—OR²²; —C(═O)—R²³; —S(═O)—R²⁴; —S(═O)₂—R²⁴, or for a linear or branched, saturated or unsaturated, unsubstituted or mono- or polysubstituted aliphatic C₁₋₁₀ residue;
R⁵ stands for H; F; Cl; Br; I; —SF₅; —NO₂; —CF₃; —CF₂Cl; —CN; —NH₂; —OH; —SH; —C(═O)—NH₂; —S(═O)₂—NH₂; —C(═O)—NH—OH; —C(═O)—OH; —C(═O)—H; —S(═O)₂—OH; —NHR¹¹; —NR¹²R¹³; —OR¹⁴; —SR¹⁵; —C(═O)—NHR¹⁶; —C(═O)—NR¹⁷R¹⁸; —S(═O)₂—NHR¹⁹; —S(═O)₂—NR²⁰R²¹; —C(═O)—OR²²; —C(═O)—R²³, —S(═O)₂—R²⁴; —S(═O)—R²⁴; or
for a linear or branched, saturated or unsaturated, unsubstituted or mono- or polysubstituted aliphatic C₁₋₁₀ residue; or
for an unsaturated or saturated, unsubstituted or mono- or polysubstituted 3-, 4-, 5-, 6-, 7-, 8- or 9-membered cycloaliphatic residue, which optionally may contain one or more heteroatoms as ring members and is respectively bonded to the basic framework via a carbon atom in the ring of the cycloaliphatic residue;
R⁶ and R⁷ each independently stand for H; F; Cl; Br; I; —SF₅; —NO₂; —CF₃; —CF₂Cl; —CN; —NH₂; —OH; —SH; —C(═O)—NH₂; —S(═O)₂—NH₂; —C(═O)—NH—OH; —C(═O)—OH; —C(═O)—H; —S(═O)₂—OH; —NHR¹¹; —NR¹²R¹³; —OR¹⁴; —SR¹⁵; —C(═O)—NHR¹⁶; —C(═O)—NR¹⁷R¹⁸; —S(═O)₂—NHR¹⁹; —S(═O)₂—NR²⁰R²¹; —C(═O)—OR²²; —C(═O)—R²³, —S(═O)—R²⁴; —S(═O)₂—R²⁴;
or for a linear or branched, saturated or unsaturated, unsubstituted or mono- or polysubstituted aliphatic C₁₋₁₀ residue; or
for an unsubstituted or mono- or polysubstituted 6- or 10-membered aryl residue, which optionally may be bonded via a linear or branched, unsubstituted or mono- or polysubstituted C₁₋₆-alkylene group or C₁₋₂₋₆-alkenylene group or C₂₋₆-alkinylene group;
R⁹ stands for H; F; Cl; Br; I; —SF₅; —NO₂; —CF₃; —CF₂Cl; —CN; —NH₂; —OH; —SH; —C(═O)—NH₂; —S(═O)₂—NH₂; —C(═O)—NH—OH; —C(═O)—OH; —C(═O)—H; —S(═O)₂—OH; —NHR¹¹; —NR¹²R¹³; —OR¹⁴; —SR¹⁵; —C(═O)—NHR¹⁶; —C(═O)—NR¹⁷R¹⁸; —S(═O)₂—NHR¹⁹; —S(═O)₂—NR²⁰R²¹; —C(═O)—OR²²; —C(═O)—R²³; —S(═O)—R²⁴; —S(═O)₂—R²⁴ or for a linear or branched, saturated or unsaturated, unsubstituted or mono- or polysubstituted aliphatic C₁₋₁₀ residue;
R¹¹, R¹², R¹³, R¹⁴, R¹⁵, R¹⁶, R¹⁷, R¹⁸, R¹⁹, R²⁰, R²¹, R²², R²³ and R²⁴ each independently stand for a linear or branched, saturated or unsaturated, unsubstituted or mono- or polysubstituted aliphatic Cl₁₀ residue; or
for an unsaturated or saturated, unsubstituted or mono- or polysubstituted 3-, 4-, 5-, 6-, 7-, 8- or 9-membered cycloaliphatic residue, which optionally may contain one or more heteroatoms as ring members and which optionally may be condensed with a saturated or unsaturated, unsubstituted or mono- or polysubstituted mono- or polycyclic ring system and/or optionally may be bonded via a linear or branched, unsubstituted or mono- or polysubstituted C₁₋₆-alkylene group or 2- to 6-membered heteroalkylene group; or
for an unsubstituted or mono- or polysubstituted 5- to 14-membered aryl or heteroaryl residue, which optionally may be condensed with a saturated or unsaturated, unsubstituted or mono- or polysubstituted mono- or polycyclic ring system and/or optionally may be bonded via a linear or branched, unsubstituted or mono- or polysubstituted C₁₋₆-alkylene group or 2- to 6-membered heteroalkylene group; or
R¹² and R¹³ together with the nitrogen atom connecting them form a saturated or unsaturated, unsubstituted or mono- or polysubstituted 4-, 5-, 6-, 7-, 8- or 9-membered heterocycloaliphatic residue, which optionally may contain one or more further heteroatoms as ring members and which optionally may be condensed with a saturated or unsaturated, unsubstituted or mono- or polysubstituted mono- or polycyclic ring system;
K, L and M each independently stand for H, F, Cl, Br, I, —CN, —CF₃, —SF₅, —OH, —O—C₁₋₅-alkyl, —NH₂, —NO₂, —O—CF₃, —S—CF₃, —SH, —S—C₁₋₅-alkyl, —C₁₋₅-alkyl, —C(═O)—OH, —C(═O)—O—C₁₋₅-alkyl, —NH—C₁₋₅-alkyl, —N(C₁₋₅-alkyl)₂, —NH—S(═O)₂—C₁₋₅-alkyl, —NH—C(═O)—O—C₁₋₅-alkyl, —C(═O)—H, —C(═O)—C₁₋₅-alkyl, —C(═O)—NH₂, —C(═O)—NH—C₁₋₅-alkyl, —C(═O)—N—(C₁₋₅-alkyl)₂, —O-phenyl, —O-benzyl, phenyl and benzyl, wherein the cyclic part of the —O-phenyl, —O-benzyl, phenyl and benzyl residues may each optionally be substituted with 1, 2, 3, 4 or 5 substituents independently selected from the group consisting of F, Cl, Br, —OH, —CF₃, —SF₅, —CN, —NO₂, —C₁₋₅-alkyl, —O—C₁₋₅-alkyl, —O—CF₃, —S—CF₃, phenyl and —O-benzyl;
R²⁵ and R²⁶ each independently stand for hydrogen; or
for a linear or branched, saturated or unsaturated, unsubstituted or mono- or polysubstituted aliphatic C₁₋₁₀ residue; or
for an unsubstituted or mono- or polysubstituted 5- to 14-membered aryl or heteroaryl residue, which optionally may be condensed with a saturated or unsaturated, unsubstituted or mono- or polysubstituted mono- or polycyclic ring system and/or optionally may be bonded via a linear or branched, unsubstituted or mono- or polysubstituted C₁₋₆-alkylene group or C₂₋₆-alkenylene group or C₂₋₆-alkinylene group; or
for an unsaturated or saturated, unsubstituted or mono- or polysubstituted 3-, 4-, 5-, 6-, 7-, 8- or 9-membered cycloaliphatic residue, which optionally may contain one or more heteroatoms as ring members;
with the proviso that R²⁵ and R²⁶ do not simultaneously stand for hydrogen;
or
R²⁵ and R²⁶ together with the carbon atom connecting them form a saturated or unsaturated, unsubstituted or mono- or polysubstituted 3-, 4-, 5- or 6-membered cycloaliphatic residue; and
R³⁴, R³⁵, R³⁶ and R³⁷ each independently stand for hydrogen, or for a linear or branched, saturated or unsaturated aliphatic C₁₋₁₀ residue; or
for an unsaturated or saturated, unsubstituted or mono- or polysubstituted 3-, 4-, 5-, 6-, 7-, 8- or 9-membered cycloaliphatic residue, which is respectively bonded to the basic framework via a carbon atom in the ring of the cycloaliphatic residue and which optionally may be condensed with a saturated or unsaturated, unsubstituted or mono- or polysubstituted mono- or polycyclic ring system and/or optionally may be bonded via a linear or branched, unsubstituted or mono- or polysubstituted C₁₋₆-alkylene group or C₂₋₆-alkenylene group or C₂₋₆-alkinylene group; or
for an unsubstituted or mono- or polysubstituted 5- to 14-membered aryl or heteroaryl residue, which optionally may be condensed with a saturated or unsaturated, unsubstituted or mono- or polysubstituted mono- or polycyclic ring system and/or optionally may be bonded via a linear or branched, unsubstituted or mono- or polysubstituted C₁₋₆-alkylene group or C₂₋₆-alkenylene group or C₂₋₆-alkinylene group;
or a pharmaceutically acceptable salt thereof.

5. A method as claimed in claim 4, wherein said compound is in the form of an individual stereoisomer.

6. A method as claimed in claim 4, wherein said compound is in the form of a mixture of stereoisomers in any mixing ratio.

7. A method as claimed in claim 6, wherein said compound is in the form of a racemic mixture.

8. A method as claimed in claim 4, wherein in said compound corresponding to formula I
X stands for O;
W stands for —NR³⁴R³⁵;
n stands for 1;
R¹, R³ and R⁴ each stand for H;
R² stands for methyl; —O—CH₃; F; Cl; Br or I;
R⁵ stands for a residue selected from the group consisting of methyl, ethyl, —CF₃, —CCl₃, —CBr₃, —CHF₂, —CH₂F, —CF₂Cl, —CCl₂F, —C(CH₃)₂(CH₂OH), tert-butyl, —O—CF₃, —O—CCl₃, —O—CBr₃, —O—CHF₂, —O—CH₂F, —S—CF₃, —S—CCl₃, —S—CBr₃, —S—CHF₂ and —S—CH₂F; or
for a residue selected from the group consisting of cyclopropyl, cyclobutyl, cyclopentyl, cyclohexyl, cyclopentenyl and cyclohexenyl;
T stands for CH and U for CH and V for N;
or
T stands for CH and U for N and V for CH;
or
T stands for N and U for CH and V for CH;
or
T stands for N and U for N and V for CH;
or
T stands for N and U for CH and V for N;
or
T stands for CH and U for N and V for N;
or
T stands for CH and U for CH and V for CH;
R²⁵ stands for an alkyl residue selected from the group consisting of —CH₂—OH, —CH₂—CH₂—OH, methyl, ethyl and n-propyl or for a residue selected from the group consisting of benzyl, phenyl, phenethyl, cyclopropyl, cyclobutyl, cyclopentyl and cyclohexyl;
R²⁶ stands for hydrogen or for a residue selected from the group consisting of methyl, ethyl and n-propyl; or
R²⁵ and R²⁶ together with the carbon atom connecting them form a residue selected from the group consisting of cyclopropyl, cyclobutyl, cyclopentyl and cyclohexyl; and
R³⁴ and R³⁵ each independently stand for hydrogen, or for a linear or branched, saturated or unsaturated aliphatic C₁₋₁₀ residue.

9. A method as claimed in claim 4, wherein said compound is selected from the group consisting of:
N-((2-(4-(dimethylamino)-4-phenylpiperidin-1-yl)-6-(trifluoromethyl)pyridin-3-yl)methyl)-2-(3-fluoro-4-(methylsulfonamido)phenyl)propanamide;
N-((6-tert-butyl-2-(4-(dimethylamino)-4-phenylpiperidin-1-yl)pyridin-3-yl)methyl)-2-(3-fluoro-4-(methylsulfonamido)phenyl)propanamide;
N-((2-(4-benzyl-4-(dimethylamino)piperidin-1-yl)-6-(trifluoromethyl)pyridin-3-yl)methyl)-2-(3-fluoro-4-(methylsulfonamido)phenyl)propanamide;
N-((2-(4-benzyl-4-(dimethylamino)piperidin-1-yl)-6-tert-butylpyridin-3-yl)methyl)-2-(3-fluoro-4-(methylsulfonamido)phenyl)propanamide;
N-(2-(4-benzyl-4-(dimethylamino)piperidin-1-yl)-4-(trifluoromethyl)benzyl)-2-(3-fluoro-4-(methylsulfonamido)phenyl)propanamide;
N-(2-(4-benzyl-4-(dimethylamino)piperidin-1-yl)-4-(trifluoromethyl)benzyl)-2-(3-methyl-4-(methylsulfonamido)phenyl)propanamide;
N-((2-(4-(dimethylamino)-4-(3-fluorobenzyl)piperidin-1-yl)-6-(trifluoromethyl)pyridin-3-yl)methyl)-2-(3-fluoro-4-(methylsulfonamido)-phenyl)propanamide;
N-(2-(4-(dimethylamino)-4-(3-fluorobenzyl)piperidin-1-yl)-4-(trifluoromethyl)-benzyl)-2-(3-fluoro-4-(methylsulfonamido)phenyl)propanamide;
N-(2-(4-(dimethylamino)-4-(3-fluorobenzyl)piperidin-1-yl)-4-(trifluoromethyl)-benzyl)-2-(3-methyl-4-(methylsulfonamido)phenyl)propanamide; and
pharmaceutically acceptable salts of any of the foregoing compounds.

10. A method as claimed in claim 3, wherein said compound corresponds to formula V: 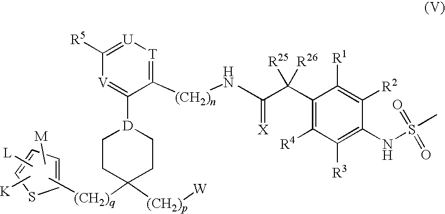
wherein
D is CH or N;
W stands for —CN, —NR³⁴R³⁵, —C(═O)—R³⁶ or —C(═O)—OR³⁷;
X stands for O, S or N—C≡N;
n stands for 0, 1, 2, 3 or 4;
p is 0, 1, 2 or 3;
q is 0, 1, 2 or 3;
T stands for C—R⁶ and U for C—R⁷ and V for N;
or
T stands for C—R⁶ and U for N and V for C—R⁹;
or
T stands for N and U for C—R⁷ and V for C—R⁹;
or
T stands for N and U for N and V for C—R⁹;
or
T stands for N and U for C—R⁷ and V for N;
or
T stands for C—R⁶ and U for N and V for N;
or
T stands for C—R⁶ and U for C—R⁷ and V for C—R⁹;
R¹, R², R³ and R⁴ each independently stand for H; F; Cl; Br; I; —SF₅; —NO₂; —CF₃; —CN; —NH₂; —OH; —SH; —C(═O)—NH₂; —S(═O)₂—NH₂; —C(═O)—NH—OH; —C(═O)—OH; —C(═O)—H; —S(═O)₂—OH; —NHR¹¹; —NR¹²R¹³; —OR¹⁴; —SR¹⁵; —C(═O)—NHR¹⁶; —C(═O)—NR¹⁷R¹⁸; —S(═O)₂—NHR¹⁹; —S(═O)₂—NR²⁰R²¹; —C(═O)—OR²²; —C(═O)—R²³; —S(═O)—R²⁴; —S(═O)₂—R²⁴ or for a linear or branched, saturated or unsaturated, unsubstituted or mono- or polysubstituted aliphatic Cl₁₀ residue;
R⁵ stands for H; F; Cl; Br; I; —SF₅; —NO₂; —CF₃; —CF₂Cl; —CN; —NH₂; —OH; —SH; —C(═O)—NH₂; —S(═O)₂—NH₂; —C(═O)—NH—OH; —C(═O)—OH; —C(═O)—H; —S(═O)₂—OH; —NHR¹¹; —NR¹²R¹³; —OR¹⁴; —SR¹⁵; —C(═O)—NHR¹⁶; —C(═O)—NR¹⁷R¹⁸; —S(═O)₂—NHR¹⁹; —S(═O)₂—NR²⁰R²¹; —C(═O)—OR²²; —C(═O)—R²³, —S(═O)₂—R²⁴; —S(═O)—R²⁴; or
for a linear or branched, saturated or unsaturated, unsubstituted or mono- or polysubstituted aliphatic Cl₁₀ residue; or
for an unsaturated or saturated, unsubstituted or mono- or polysubstituted 3-, 4-, 5-, 6-, 7-, 8- or 9-membered cycloaliphatic residue, which optionally may contain one or more heteroatoms as ring member and is respectively bonded to the basic framework via a carbon atom in the ring of the cycloaliphatic residue;
R⁶ and R⁷ each independently stand for H; F; Cl; Br; I; —SF₅; —NO₂; —CF₃; —CF₂Cl; —CN; —NH₂; —OH; —SH; —C(═O)—NH₂; —S(═O)₂—NH₂; —C(═O)—NH—OH; —C(═O)—OH; —C(═O)—H; —S(═O)₂—OH; —NHR¹¹; —NR¹²R¹³; —OR¹⁴; —SR¹⁵; —C(═O)—NHR¹⁶; —C(═O)—NR¹⁷R¹⁸; —S(═O)₂—NHR¹⁹; —S(═O)₂—NR²⁰R²¹; —C(═O)—OR²²; —C(═O)—R²³, S(═O)—R²⁴; —S(O)₂—R²⁴;
or for a linear or branched, saturated or unsaturated, unsubstituted or mono- or polysubstituted aliphatic C₁₋₁₀ residue; or
for an unsubstituted or mono- or polysubstituted 6- or 10-membered aryl residue, which can be bonded via a linear or branched, unsubstituted or mono- or polysubstituted C₁₋₆-alkylene group or C₂₋₆-alkenylene group or C₂₋₆-alkinylene group;
R⁹ stands for H; F; Cl; Br; I; —SF₅; —NO₂; —CF₃; —CF₂Cl; —CN; —NH₂; —OH; —SH; —C(═O)—NH₂; —S(═O)₂—NH₂; —C(═O)—NH—OH; —C(═O)—OH; —C(═O)—H; —S(═O)₂—OH; —NHR¹¹; —NR¹²R¹³; —OR¹⁴; —SR¹⁵; —C(═O)—NHR¹⁶; —C(═O)—NR¹⁷R¹⁸; —S(═O)₂—NR¹⁹; —S(═O)₂—NR²⁰R²¹; —C(═O)—OR²²; —C(═O)—R²³; —S(═O)—R²⁴; —S(═O)₂—R²⁴ or for a linear or branched, saturated or unsaturated, unsubstituted or mono- or polysubstituted aliphatic Cl₁₀ residue;
R¹¹, R¹², R¹³, R¹⁴, R¹⁵, R¹⁶, R¹⁷, R¹⁸, R¹⁹, R²⁰, R²¹, R²², R²³ and R²⁴ each independently stand for a linear or branched, saturated or unsaturated, unsubstituted or mono- or polysubstituted aliphatic Cl₁₀ residue; or
for an unsaturated or saturated, unsubstituted or mono- or polysubstituted 3-, 4-, 5-, 6-, 7-, 8- or 9-membered cycloaliphatic residue, which optionally may contain one or more heteroatoms as ring members and which optionally may be condensed with a saturated or unsaturated, unsubstituted or mono- or polysubstituted mono- or polycyclic ring system and/or optionally may be bonded via a linear or branched, unsubstituted or mono- or polysubstituted C₁₋₆-alkylene group or 2- to 6-membered heteroalkylene group; or
for an unsubstituted or mono- or polysubstituted 5- to 14-membered aryl or heteroaryl residue, which optionally may be condensed with a saturated or unsaturated, unsubstituted or mono- or polysubstituted mono- or polycyclic ring system and/or optionally may be bonded via a linear or branched, unsubstituted or mono- or polysubstituted C₁₋₆-alkylene group or 2- to 6-membered heteroalkylene group; or
R¹² and R¹³ together with the nitrogen atom connecting them form a saturated or unsaturated, unsubstituted or mono- or polysubstituted 4-, 5-, 6-, 7-, 8- or 9-membered heterocycloaliphatic residue, which possibly has at least one further heteroatom as ring member and which optionally may be condensed with a saturated or unsaturated, unsubstituted or mono- or polysubstituted mono- or polycyclic ring system;
K, L and M each independently stand for H, F, Cl, Br, I, —CN, —CF₃, —SF₅, —OH, —O—C₁₋₅-alkyl, —NH₂, —NO₂, —O—CF₃, —S—CF₃, —SH, —S—C₁₋₅-alkyl, —C₁₋₅-alkyl, —C(═O)—OH, —C(═O)—O—C₁₋₅-alkyl, —NH—C₁₋₅-alkyl, —N(C₁₋₅-alkyl)₂, —NH—S(═O)₂—C₁₋₅-alkyl, —NH—C(═O)—O—C₁₋₅-alkyl, —C(═O)—H, —C(═O)—C₁₋₅-alkyl, —C(═O)—NH₂, —C(═O)—NH—C₁₋₅-alkyl, —C(═O)—N—(C₁₋₅-alkyl)₂, —O-phenyl, —O-benzyl, phenyl and benzyl, wherein the cyclic part of the residues —O-phenyl, —O-benzyl, phenyl and benzyl can be respectively substituted with 1, 2, 3, 4 or 5 substituents independently selected from the group consisting of F, Cl, Br, —OH, —CF₃, —SF₅, —CN, —NO₂, —C₁₋₅-alkyl, —O—C₁₋₅-alkyl, —O—CF₃, —S—CF₃, phenyl and —O-benzyl;
R²⁵ and R²⁶ each independently stand for hydrogen; or
for a linear or branched, saturated or unsaturated, unsubstituted or mono- or polysubstituted aliphatic C₁₋₁₀ residue; or
for an unsubstituted or mono- or polysubstituted 5- to 14-membered aryl or heteroaryl residue, which optionally may be condensed with a saturated or unsaturated, unsubstituted or mono- or polysubstituted mono- or polycyclic ring system and/or optionally may be bonded via a linear or branched, unsubstituted or mono- or polysubstituted C₁₋₆-alkylene group or C₁₋₂₋₆-alkenylene group or C₂₋₆-alkinylene group; or
for an unsaturated or saturated, unsubstituted or mono- or polysubstituted 3-, 4-, 5-, 6-, 7-, 8- or 9-membered cycloaliphatic residue, which optionally may contain one or more heteroatoms as ring members;
with the proviso that R²⁵ and R²⁶ do not simultaneously stand for hydrogen;
or
R²⁵ and R²⁶ together with the carbon atom connecting them form a saturated or unsaturated, unsubstituted or mono- or polysubstituted 3-, 4-, 5- or 6-membered cycloaliphatic residue; and
R³⁴, R³⁵, R³⁶ and R³⁷ each independently stand for hydrogen, or for a linear or branched, saturated or unsaturated aliphatic C₁₋₁₀ residue; or
for an unsaturated or saturated, unsubstituted or mono- or polysubstituted 3-, 4-, 5-, 6-, 7-, 8- or 9-membered cycloaliphatic residue, which is respectively bonded to the basic framework via a carbon atom in the ring of the cycloaliphatic residue and which optionally may be condensed with a saturated or unsaturated, unsubstituted or mono- or polysubstituted mono- or polycyclic ring system and/or optionally may be bonded via a linear or branched, unsubstituted or mono- or polysubstituted C₁₋₆-alkylene group or C₂₋₆-alkenylene group or C₂₋₆-alkinylene group; or
for an unsubstituted or mono- or polysubstituted 5- to 14-membered aryl or heteroaryl residue, which optionally may be condensed with a saturated or unsaturated, unsubstituted or mono- or polysubstituted mono- or polycyclic ring system and/or optionally may be bonded via a linear or branched, unsubstituted or mono- or polysubstituted C₁₋₆-alkylene group or C₂₋₆-alkenylene group or C₂₋₆-alkinylene group;
or a pharmaceutically acceptable salt thereof.

11. A method as claimed in claim 10, wherein said compound is in the form of an individual stereoisomer.

12. A method as claimed in claim 10, wherein said compound is in the form of a mixture of stereoisomers in any mixing ratio.

13. A method as claimed in claim 12, wherein said compound is in the form of a racemic mixture.

14. A method as claimed in claim 10, wherein in said compound corresponding to formula V
X stands for O;
W stands for —NR³⁴R³⁵;
n stands for 1;
R¹, R³ and R⁴ respectively stand for H;
R² stands for methyl; —O—CH₃; F; Cl; Br or I;
R⁵ stands for a residue selected from the group consisting of methyl, ethyl, —CF₃, —CCl₃, —CBr₃, —CHF₂, —CH₂F, —CF₂Cl, —CCl₂F, —C(CH₃)₂(CH₂OH), tert-butyl, —O—CF₃, —O—CCl₃, —O—CBr₃, —O—CHF₂, —O—CH₂F, —S—CF₃, —S—CCl₃, —S—CBr₃, —S—CHF₂ and —S—CH₂F; or
for a residue selected from the group consisting of cyclopropyl, cyclobutyl, cyclopentyl, cyclohexyl, cyclopentenyl and cyclohexenyl;
T stands for CH and U for CH and V for N;
or
T stands for CH and U for N and V for CH;
or
T stands for N and U for CH and V for CH;
or
T stands for N and U for N and V for CH;
or
T stands for N and U for CH and V for N;
or
T stands for CH and U stands for N and V for N;
or
T stands for CH and U for CH and V for CH;
R²⁵ stands for an alkyl residue selected from the group consisting of —CH₂—OH, —CH₂—CH₂—OH, methyl, ethyl and n-propyl; or
for a residue selected from the group consisting of benzyl, phenyl, phenethyl, cyclopropyl, cyclobutyl, cyclopentyl and cyclohexyl;
R²⁶ stands for hydrogen or for a residue selected from the group consisting of methyl, ethyl and n-propyl; or
R²⁵ and R²⁶ together with the carbon atom connecting them form a residue selected from the group consisting of cyclopropyl, cyclobutyl, cyclopentyl and cyclohexyl; and
R³⁴ and R³⁵ each independently stand for hydrogen, or for a linear or branched, saturated or unsaturated aliphatic C₁₋₁₀ residue.

15. A method as claimed in claim 10, wherein said compound is selected from the group consisting of:
N-((2-(4-(dimethylamino)-4-(thiophen-2-yl)piperidin-1-yl)-6-(trifluoromethyl)pyridin-3-yl)methyl)-2-(3-fluoro-4-(methylsulfonamido)phenyl)propanamide;
N-(2-(4-(dimethylamino)-4-(thiophen-2-yl)piperidin-1-yl)-4-(trifluoromethyl)benzyl)-2-(3-fluoro-4-(methylsulfonamido)phenyl)propanamide, and
pharmaceutically acceptable salts of any of the foregoing compounds.

16. A method as claimed in claim 1, wherein the μ-opioid receptor affinity expressed in terms of human Kᵢ value is ≦1.0 μM, and the VR1 receptor affinity expressed in terms of human Kᵢ value is ≦1.0 μM.

17. A method as claimed in claim 16, wherein the μ-opioid receptor affinity expressed in terms of human Kᵢ value is ≦100 nM, and the VR1 receptor affinity expressed in terms of human Kᵢ value is ≦100 nM.

18. A method as claimed in claim 17, wherein the μ-opioid receptor affinity expressed in terms of human Kᵢ value is ≦10 nM, and the VR1 receptor affinity expressed in terms of human Kᵢ value is ≦10 nM.

19. A method as claimed in claim 18, wherein the μ-opioid receptor affinity expressed in terms of human Kᵢ value is ≦1.0 nM, and the VR1 receptor affinity expressed in terms of human Kᵢ value is ≦1.0 nM.

20. A method as claimed in claim 1, wherein said pain is selected from the group consisting of acute pain, chronic pain, or neuropathic pain.

21. A method as claimed in claim 1, wherein said subject is a patient suffering from post-zoster neuralgia, a patient with increased opioid addiction potential, a patient with opioid-induced hyperalgesia, a patient who has developed a tolerance to opioid analgesics, a patient with allodynia, a patient with diabetic neuropathy, a patient with post-operative pain, a patient with a psychological disorder or a patient over 60 years of age.

22. A method as claimed in claim 1, for producing narcosis or analgesia during narcosis or both narcosis and analgesia during narcosis.

23. A method a claimed in claim 1, wherein the compound having affinity to the VR1 receptor act as an antagonist, an inverse agonist or partial antagonist on VR1 receptors.

24. A method as claimed in claim 1, wherein the compound having affinity to the μ-opioid receptor act as an agonist, partial agonist or mixed agonist-antagonist on opioid receptors.

25. A method as claimed in claim 1, wherein said compound or compounds is/are administered as an injectable solution, drops or juice, tablets, patches, capsules, plasters or an aerosol.

26. A method as claimed in claim 1, wherein said compound or compounds is/are administered in a multi-particulate form selected from the group consisting of granules, pellets, active substance crystals or microparticles, and wherein said multi-particulate is compressed into tablets or incorporated into capsules.

27. A method of treating a condition selected from the group consisting of overactive bladder syndrome, coughing, asthma, chronic obstructive pulmonary disease, and diabetes in a subject, said method comprising administering to said subject a pharmaceutically effective amount of:
i) a combination comprising at least one compound that has affinity to the μ-opioid receptor and at least one compound that has affinity to the VR1 receptor, or
ii) at least one compound that has affinity to the μ-opioid receptor and the VR1 receptor;
wherein the μ-opioid receptor affinity expressed in terms of human Kᵢ value is ≦5.0 μM, and the VR1 receptor affinity expressed in terms of human Kᵢ value is ≦5.0 μM.
